SEQUENCE LISTING (80 sequences)

SEQ ID NO: 1 - protein, 116 aa, Streptococcus pyogenes
GAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 2 - DNA, 348 bp, Streptococcus pyogenes
ggtgcaatggttgataccctgagcggtctgagcagcgaacagggtcagagcggtgatatgaccattgaagaagatagcgcaacccacatcaaattcagcaaacgtgatgaagatggtaaagaactggcaggcgcaacaatggaactgcgtgatagcagcggtaaaaccattagcacctggattagtgatggtcaggtgaaagatttttatctgtaccctggcaaatacacctttgttgaaaccgcagcaccggatggttatgaagttgcaaccgcaattacctttaccgttaatgaacagggccaggttaccgtgaatggtaaagcaaccaaaggtgatgcacatatt

SEQ ID NO: 3 - protein, 104 aa, Streptococcus dysgalactiae
IDTMSGLSGETGQSGNTTIEEDSTTHVKFSKRDSNGKELAGAMIELRNLSGQTIQSWVSDGTVKDFYLMPGTYQFVETAAPEGYELAAPITFTIDEKGQIWVDS

SEQ ID NO: 4 - DNA, 318 bp, Streptococcus dysgalactiae
atgggtattgataccatgagcggtctgagcggtgaaaccggtcagagcggtaataccaccattgaagaggatagcaccacacatgtgaaattcagcaaacgcgatgcaaacggcaaagaactggcaggcgcaatgattgaactgcgtaatctgagtggtcagaccattcagagctgggttagtgatggcaccgttaaagatttttatctgatgcctggcacctatcagtttgttgaaaccgcagcaccggaaggttatgagctggcagcaccgattacctttaccattgatgaaaaaggtcagatttgggttgatagc

SEQ ID NO: 5 - protein, 13 aa, Streptococcus pyogenes
AHIVMVDAYKPTK

SEQ ID NO: 6 - DNA, 39 bp, Streptococcus pyogenes
gctcacatcgtgatggtggacgcttacaagcccaccaag

SEQ ID NO: 7 - protein, 13 aa, Streptococcus dysgalactiae
DPIVMIDNDKPIT

SEQ ID NO: 8 - DNA, 39 bp, Streptococcus dysgalactiae
gaccccatcgtgatgatcgacaacgacaagcccatcacc

SEQ ID NO: 9 - protein, 123 aa, Streptococcus pneumoniae
SSGLVPRGSHMKPLRGAVFSLQKQHPDYPDIYGAIDQNGTYQNVRTGEDGKLTFKNLSDGKYRLFENSEPAGYKPVQNKPIVAFQIVNGEVRDVTSIVPQDIPATYEFTNGKHYITNEPIPPK

SEQ ID NO: 10 - DNA, 369 bp, Streptococcus pneumoniae
agcagcggcctggtgccgcgcggcagccatatgaagccgctgcgtggtgccgtgtttagcctgcagaaacagcatcccgactatcccgatatctatggcgcgattgatcagaatgggacctatcaaaatgtgcgtaccggcgaagatggtaaactgacctttaagaatctgagcgatggcaaatatcgcctgtttgaaaatagcgaacccgctggctataaaccggtgcagaataagccgattgtggcgtttcagattgtgaatggcgaagtgcgtgatgtgaccagcattgtgccgcaggatattccggctacatatgaatttaccaacggtaaacattatatcaccaatgaaccgataccgccgaaa

SEQ ID NO: 11 - DNA, 57 bp, Bacillus cereus
gaaaaagtgatgggccagttcgaaatcatgaaagttgatgccaacgacaagaccaaa

SEQ ID NO: 12 - protein, 19 aa, Bacillus cereus
EKVMGQFEIMKVDANDKTK

SEQ ID NO: 13 - protein, 129 aa, Actinomyces viscosus
GSLSKYGKVILTKTGTDDLADKTKYNGAQFQVYECTKTASGATLRDSDPSTQTVDPLTIGGEKTFTTAGQGTVEINYLRANDYVNGAKKDQLTDEDYYCLVETKAPEGYNLQADPLPFRVLAEKAEKKA

SEQ ID NO: 14 - DNA, 387 bp, Actinomyces viscosus
ggtagcctgagcaaatatggtaaagtgattctgaccaaaaccggcaccgatgatctggcagataaaaccaaatataacggtgcacagtttcaggtgtatgaatgtaccaaaacagcaagcggtgcaaccctgcgtgatagcgatccgagcacacagaccgttgatccgctgaccattggtggtgaaaaaacctttaccaccgcaggtcagggcaccgttgaaattaattatctgcgtgccaatgattatgtgaacggtgcaaaaaaagatcagctgaccgatgaagattattactgtctggttgaaaccaaagcaccggaaggttataatctgcaggcagatccgctgccgtttcgtgttctggccgaaaaagcagaaaaaaaagcc

SEQ ID NO: 15 - protein, 102 aa, Streptococcus pneumoniae
GSTTKVKLIKVDQDHNRLEGVGFKLVSVARDVSAAAVPLIGEYRYSSSGQVGRTLYTDKNGEIFVTNLPLGNYRFKEVEPLAGYAVTTLDTDVQLVDHQLVT

SEQ ID NO: 16 - DNA, 306 bp, Streptococcus pneumoniae
ggtagcaccaccaaagtgaaactgattaaagttgatcaggatcacaatcgtctggaaggtgttggttttaaactggttagcgttgcacgtgatgttagcgcagcagcagttccgctgattggtgaatatcgttatagcagcagcggtcaggttggtcgtaccctgtataccgataaaaatggcgaaattttcgttaccaatctgccgctgggtaactatcgttttaaagaagttgaaccgctggcaggttatgcagttaccacactggataccgatgttcagctggttgatcatcagctggtgacc

SEQ ID NO: 17 - protein, 94 aa, Streptococcus pneumoniae
PRGNVDFMKVDGRTNTSLQGAMFKVMKEESGHYTPVLQNGKEVVVTSGKDGRFRVEGLEYGTYYLWELQAPTGYVQLTSPVSFTIGKDTRKELV

SEQ ID NO: 18 - DNA, 282 bp, Streptococcus pneumoniae
ccgcgtggtaatgttgattttatgaaagttgatggtcgcaccaataccagcctgcagggtgcaatgtttaaagtgatgaaagaagaaagcggtcactatacaccggtgctgcagaatggtaaagaagttgttgttaccagcggtaaagatggtcgttttcgtgttgaaggtctggaatatggcacctattatctgtgggaactgcaggcaccgaccggttatgttcagctgaccagtccggttagttttaccattggcaaagatacccgtaaagaactggtg

SEQ ID NO: 19 - protein, 123 aa, Corynebacterium diphtheriae
VVTYHGKLKVVKKDGKEAGKVLKGAEFELYQCTSAAVLGKGPLTVDGVKKWTTGDDGTFTIDGLHVTDFEDGKEAAPATKKFCLKETKAPAGYALPDPNVTEIEFTRAKISEKDKFEGDDEVT

SEQ ID NO: 20 - DNA, 369 bp, Corynebacterium diphtheriae
gttgttacctatcatggtaaactgaaagtggtgaaaaaagacggtaaagaggcaggcaaagttctgaaaggtgcagaatttgaactgtatcagtgtaccagcgcagcagttttaggtaaaggtccgctgaccgttgatggtgtgaaaaaatggaccaccggtgatgatggcacctttaccattgatggtctgcatgttaccgattttgaagatggtaaagaagccgcaccggcaaccaaaaaattctgtctgaaagaaaccaaagcaccggcaggttatgcactgcctgatccgaatgtgaccgaaattgaatttacccgtgcaaaaatcagcgagaaagataaatttgaaggcgacgatgaagtgacc

SEQ ID NO: 21 - DNA, 51 bp, Bacillus cereus
gttaccggtcagtttgaaatcgttaaagttgatgccgaagataagacccgt

SEQ ID NO: 22 - protein, 17 aa, Bacillus cereus
VTGQFEIVKVDAEDKTR

SEQ ID NO: 23 - protein, 134 aa, Lactobacillus rhamnosus
STNDTTTQNVVLTKYGFDKDVTAIDRATDQIWTGDGAKPLQGVDFTIYNVTANYWASPKDYKGSFDSAPVAATGTTNDKGQLTQALPIQSKDASGKTRAAVYLFHETNPRAGYNTSADFWLTLPAKAAADGNVY

SEQ ID NO: 24 - DNA, 402 bp, Lactobacillus rhamnosus
agcaccaatgataccaccacacagaatgttgttctgaccaaatatggcttcgataaagatgttaccgcaattgatcgtgcaaccgatcagatttggaccggtgatggtgcaaaaccgctgcagggtgttgattttaccatttataacgtgaccgccaattattgggcaagcccgaaagattataaaggcagctttgatagcgcaccggttgcagccaccggtacaacaaatgataaaggccagctgacccaggcactgccgattcagagcaaagatgcaagcggtaaaacccgtgcagcagtttacctgtttcacgaaaccaatccgcgtgcaggttataataccagcgcagatttttggctgaccctgcctgcaaaagcagcagcagatggtaatgtttat

SEQ ID NO: 25 - protein, 114 aa, Lactobacillus rhamnosus
TTYERTFVKKDAETKEVLEGAGFKISNSDGKFLKLTDKDGQSVSIGEGFIDVLANNYRLTWVAESDATVFTSDKSGKFGLNGFADNTTTYTAVETNVPDGYDAAANTDFKADNS

SEQ ID NO: 26 - DNA, 342 bp, Lactobacillus rhamnosus
accacctatgaacgtacctttgttaaaaaagacgccgaaaccaaagaagttctggaaggcgcaggctttaaaatcagcaatagtgatggcaaattcctgaaactgaccgataaagatggtcagagcgttagcattggtgaaggttttattgatgttctggccaataactatcgtctgacctgggttgcagaaagtgatgcaaccgtttttaccagcgataaaagcggcaaatttggtctgaatggttttgcagataataccaccacctataccgcagttgaaaccaatgttccggatggttatgatgcagcagcaaacaccgatttcaaagccgataatagc

SEQ ID NO: 27 - protein, 107 aa, Streptococcus agalactiae
GQITIKKIDGSTKASLQGAIFVLKNATGQFLNFNDTNNVEWGTEANATEYTTGADGIITITGLKEGTYYLVEKKAPLGYNLLDNSQKVILGDGATDTTNSDNLLVNP

SEQ ID NO: 28 - DNA, 321 bp, Streptococcus agalactiae
ggtcagattaccatcaaaaaaatcgatggtagcaccaaagcaagcctgcagggtgcaatttttgttctgaaaaatgcaaccggtcagttcctgaattttaacgataccaataatgttgaatggggcaccgaagcaaatgccaccgaatataccaccggtgcagatggtattattaccattaccggtctgaaagaaggcacctattacctggttgaaaaaaaagcaccgctgggttataatctgctggataattcacagaaagtgattttaggtgatggtgcaaccgataccaccaatagcgataacctgctggttaatccg

SEQ ID NO: 29 - protein, 91 aa, Streptococcus intermedius
EQDVVFSKVNVAGEEIAGAKIQLKDAQGQVVHSWTSKAGQSETVKLKAGTYTFHEASAPTGYLAVTDITFEVDVQGKVTVKDANGNGVKAD

SEQ ID NO: 30 - DNA, 273 bp, Streptococcus intermedius
gaacaggatgttgtgtttagcaaagttaatgttgccggtgaagaaattgcgggtgcaaaaatccagctgaaagatgcacagggtcaagttgttcatagctggaccagcaaagcaggtcagagcgaaaccgttaaactgaaagcaggcacctatacctttcatgaagcaagcgcaccgaccggttatctggcagttaccgatattacctttgaagttgatgttcagggtaaagtgaccgttaaagatgcaaatggtaatggtgtgaaagccgac

SEQ ID NO: 31 - protein, 104 aa, Streptococcus pneumoniae
KLGDIEFIKVNKNDKKPLRGAVFSLQKQHPDYPDIYGAIDQNGTYQNVRTGEDGKLTFKNLSDGKYRLFENSEPAGYKPVQNKPIVAFQIVNGEVRDVTSIVPQ

SEQ ID NO: 32 - DNA, 312 bp, Streptococcus pneumoniae
aaactgggtgatattgagttcatcaaagtgaacaaaaacgataaaaaaccgctgcgtggtgcagtttttagcctgcagaaacagcatccggattacccggatatttatggtgcaattgatcagaatggcacctatcagaatgttcgtaccggtgaagatggtaaactgacctttaaaaacctgagcgacggtaaatatcgcctgtttgaaaatagcgaaccggcaggttataaaccggttcagaataaaccgattgtggcctttcagattgttaatggtgaagttcgtgatgtgaccagcattgttccgcag

SEQ ID NO: 33 - protein, 130 aa, Corynebacterium diphtheriae
GSERKGSLTLHKKKGAESEKRATGKEMDDVAGEPLNGVTFKITKLNFDLQNGDWAKFPKTAADAKGHETSTTKEVETSGNGTAVFDNLDLGIYLVEETKAPDGIVTGAPFIVSIPMVNEASDAWNYNVVA

SEQ ID NO: 34 - DNA, 390 bp, Corynebacterium diphtheriae
ggtagcgaacgtaaaggtagtctgaccctgcataaaaagaaaggtgcagaaagcgaaaaacgtgcaaccggtaaagaaatggatgatgttgccggtgaaccgctgaatggtgttacctttaaaatcaccaaactgaacttcgatctgcagaatggtgattgggcaaaatttccgaaaaccgcagcagatgcaaaaggtcatgaaaccagcaccaccaaagaagtggaaaccagcggtaatggcaccgcagtttttgataatctggatctgggtatttacctggtggaagaaaccaaagcaccggatggtattgttacaggtgcaccgtttattgttagcattccgatggttaatgaagcaagtgatgcctggaattataacgttgttgca

SEQ ID NO: 35 - protein, 16 aa, Bacillus cereus
LGQFEIVKVDSQDKTK

SEQ ID NO: 36 - DNA, 48 bp, Bacillus cereus
ctgggccagtttgaaattgttaaagttgatagccaggataaaaccaaa

SEQ ID NO: 37 - protein, 113 aa, Artificial sequence
  modified binding partner QueenCatcher
  MISC_FEATURE (1)..(113): QueenCatcher
IDTMSGLSGETGQSGNTTIEEDSTTHVKFSKRDSNGKELAGAMIELRNLSGQTIQSWVSDGTVKDFYLMPGTYQFVETAAPEGYELAAPITFTVNEQGQVTVNGKATKGDAHI

SEQ ID NO: 38 - DNA, 339 bp, Artificial sequence
  DNA encoding modified binding partner QueenCatcher
  misc_feature (1)..(339): QueenCatcher
attgataccatgagcggtctgagcggtgaaaccggtcagagcggtaataccaccattgaagaggatagcaccacacatgtgaaattcagcaaacgcgatagcaacggcaaagaactggcaggcgcaatgattgaactgcgtaatctgagtggtcagaccattcagagctgggttagtgatggcaccgttaaagatttttatctgatgcctggcacctatcagtttgttgaaaccgcagcaccggaaggttatgagctggcagcaccgattacctttaccgttaatgaacagggccaggttaccgtgaatggtaaagcaaccaaaggtgatgcacatatt

SEQ ID NO: 39 - protein, 113 aa, Artificial sequence
  modified binding partner MoonCake
  MISC_FEATURE (1)..(117): MoonCake
IDTMSGLSGETGQSGNTTIEEDSTTHVKFSKRDSNGKELAGAMIELRNLSGQTIQSWVSDGTVKDFYLMPGTYQFVETAAPEGYELAAPITFTVQDNGEVIIQGRLTRGDVHI

SEQ ID NO: 40 - DNA, 339 bp, Artificial sequence
  DNA encoding modified binding partner MoonCake
attgataccatgagcggtctgagcggtgaaaccggtcagagcggtaataccaccattgaagaggatagcaccacacatgtgaaattcagcaaacgcgatagcaacggcaaagaactggcaggcgcaatgattgaactgcgtaatctgagtggtcagaccattcagagctgggttagtgatggcaccgttaaagatttttatctgatgcctggcacctatcagtttgttgaaaccgcagcaccggaaggttatgagctggcagcaccgattacctttaccgttcaggataacggcgaagttattattcagggccgcctgacacgtggcgatgttcatatt

SEQ ID NO: 41 - protein, 113 aa, Artificial sequence
  modified binding partner Kat I
  MISC_FEATURE (1)..(117): Kat I
IDTMSGLSGETGQSGNTTIEEDSTTHVKFSKRDSNGKELAGAMIELRNLSGQTIQSWVSDGTVKDFYLMPGTYQFVETAAPEGYELAAPITFTVQDNGEVQIQGKATRGDVPI

SEQ ID NO: 42 - DNA, 339 bp, Artificial sequence
  DNA encoding modified binding partner Kat I
attgataccatgagcggtctgagcggtgaaaccggtcagagcggtaataccaccattgaagaggatagcaccacacatgtgaaattcagcaaacgcgatagcaacggcaaagaactggcaggcgcaatgattgaactgcgtaatctgagtggtcagaccattcagagctgggttagtgatggcaccgttaaagatttttatctgatgcctggcacctatcagtttgttgaaaccgcagcaccggaaggttatgagctggcagcaccgattacctttaccgttcaggataacggcgaagttcagattcagggcaaagcaacacgtggcgatgttccgatt

SEQ ID NO: 43 - protein, 10 aa, Artificial sequence
  Reference motif
  MISC_FEATURE (1)..(10): Reference motif; X is independently any amino acid
  SITE (2)..(2): independently selected from any amino acid
  SITE (3)..(3): independently selected from any amino acid
  SITE (4)..(4): independently selected from any amino acid
  SITE (8)..(8): independently selected from any amino acid
  SITE (10)..(10): independently selected from any amino acid
GXXXFVMXDX

SEQ ID NO: 44 - protein, 10 aa, Artificial sequence
  Reference motif
  MISC_FEATURE (1)..(10): Reference motif; X is independently any amino acid
  SITE (2)..(2): independently selected from any amino acid
  SITE (3)..(3): independently selected from any amino acid
  SITE (4)..(4): independently selected from any amino acid
  SITE (8)..(8): independently selected from any amino acid
  SITE (10)..(10): independently selected from any amino acid
GXXXWVMXDX

SEQ ID NO: 45 - DNA, 60 bp, Ruminococcus sp.
agcgaaaacggcaacccgctgattgtgatggtggatgataccaccaaagtgaaaattagc

SEQ ID NO: 46 - protein, 20 aa, Ruminococcus sp.
SENGNPLIVMVDDTTKVKIS

SEQ ID NO: 47 - protein, 15 aa, Artificial sequence
  peptide tag RumTrunkD9NTag
  MISC_FEATURE (1)..(15): Rumtrunk D9N
GNPLIVMVNDTTKVK

SEQ ID NO: 48 - DNA, 45 bp, Artificial sequence
  DNA encoding peptide tag RumTrunkD9NTag
  misc_feature (1)..(45): Rumtrunk D9N tag
ggtaatccgctgattgtgatggtgaatgataccaccaaagtgaaa

SEQ ID NO: 49 - DNA, 66 bp, Streptococcus phocae
ctggttaccggcaccgcacatattgttatggttgataactataagccgatcgtggaaaccggtgat

SEQ ID NO: 50 - protein, 22 aa, Streptococcus phocae
LVTGTAHIVMVDNYKPIVETGD

SEQ ID NO: 51 - DNA, 60 bp, Enterococcus faecalis
accgaagttagcggtaataccattgtgatggtggataaactgaaagaagttccgcctacc

SEQ ID NO: 52 - protein, 15 aa, Enterococcus faecalis
NTIVMVDKLKEVPPT

SEQ ID NO: 53 - DNA, 51 bp, Ruminococcus sp. AM43-6
ggcaccccgctgattgtgatggtggatgataccaccaaagtggaaattagc

SEQ ID NO: 54 - protein, 17 aa, Ruminococcus sp. AM43-6
GTPLIVMVDDTTKVEIS

SEQ ID NO: 55 - DNA, 51 bp, Ruminococcus sp. Marseille-P6503
ggcaacccgctgattgtgatgattgatgaagcggaacagaaagaaattccg

SEQ ID NO: 56 - protein, 17 aa, Ruminococcus sp. Marseille-P6503
GNPLIVMIDEAEQKEIP

SEQ ID NO: 57 - DNA, 51 bp, Ruminococcus sp. AF25-19
ggcaccccgattgtgattatggtggatgaagcgaaaccgagcctgccggat

SEQ ID NO: 58 - protein, 17 aa, Ruminococcus sp. AF25-19
GTPIVIMVDEAKPSLPD

SEQ ID NO: 59 - DNA, 51 bp, Ruminococcus sp.
gcgggcggcattattgtgatgaaagataacaccaccaaagtgagcattagc

SEQ ID NO: 60 - protein, 17 aa, Ruminococcus sp.
AGGIIVMKDNTTKVSIS

SEQ ID NO: 61 - DNA, 48 bp, Ruminococcus flavefaciens
ggcaacccgattgtgaccatgattgatgatgcgaccctggtgaaaatt

SEQ ID NO: 62 - protein, 17 aa, Ruminococcus flavefaciens
GNPIVTMIDDATLVKIS

SEQ ID NO: 63 - DNA, 51 bp, Ruminococcus sp.
ggcaacagcaccattaccatggtggatgataccaccaaagtgcatattacc

SEQ ID NO: 64 - protein, 17 aa, Ruminococcus sp.
GNSTITMVDDTTKVHIT

SEQ ID NO: 65 - DNA, 45 bp, Bacillus cereus
ggtcagttcgaaattgttaaagttgatgcaaacgataaaactaaa

SEQ ID NO: 66 - protein, 20 aa, Bacillus cereus
NEKVTGQFEIVKVDANDKTK

SEQ ID NO: 67 - DNA, 57 bp, Bacillus cereus
agcaaaagcctgggccagtttgaaattgtgaaagtggatgcgcaggataaaaccaaa

SEQ ID NO: 68 - protein, 19 aa, Bacillus cereus
SKSLGQFEIVKVDAQDKTK

SEQ ID NO: 69 - protein, 12 aa, Streptococcus pneumoniae
KLGDIEFIKVNK

SEQ ID NO: 70 - DNA, 36 bp, Streptococcus pneumoniae
aaactgggtgatattgaatttattaaagttaataaa

SEQ ID NO: 71 - protein, 15 aa, Ruminococcus sp.
GNPLIVMVDDTTKVK

SEQ ID NO: 72 - DNA, 45 bp, Ruminococcus sp.
ggcaacccgctgattgtgatggtggatgataccaccaaagtgaaa

SEQ ID NO: 73 - protein, 10 aa, Artificial sequence
  Reference binding motif
  MISC_FEATURE (1)..(10): Reference binding motif
  SITE (2)..(2): independently selected from any amino acid
  SITE (3)..(3): independently selected from any amino acid
  SITE (4)..(4): independently selected from any amino acid
  SITE (8)..(8): independently selected from any amino acid
  SITE (10)..(10): independently selected from any amino acid
GXXXIVMXDX

SEQ ID NO: 74 - protein, 10 aa, Artificial sequence
  Reference binding motif
  MISC_FEATURE (1)..(10): Reference binding motif
  SITE (2)..(2): independently selected from any amino acid
  SITE (3)..(3): independently selected from any amino acid
  SITE (4)..(4): independently selected from any amino acid
  SITE (8)..(8): independently selected from any amino acid
  SITE (10)..(10): independently selected from any amino acid
GXXXYVMXDX

SEQ ID NO: 75 - protein, 13 aa, Streptococcus intermedius
GNKLTVTDQAAPS

SEQ ID NO: 76 - protein, 16 aa, Artificial sequence
  peptide tag Clib9
  MISC_FEATURE (1)..(16): Clib9
RGVPTIVMVDCYKRYK

SEQ ID NO: 77 - DNA, 39 bp, Streptococcus intermedius
ggtaataaactgaccgttaccgatcaggcagcaccgagc

SEQ ID NO: 78 - DNA, 48 bp, Artificial sequence
  DNA encoding peptide tag Clib9
  misc_feature (1)..(48): Clib9
cgtggtgttccgaccattgttatggttgattgttataaacgttataaa

SEQ ID NO: 79 - protein, 117 aa, Artificial sequence
  MoonCake cloning sequence
  MISC_FEATURE (1)..(117): MoonCake cloning sequence
FSMGIDTMSGLSGETGQSGNTTIEEDSTTHVKFSKRDSNGKELAGAMIELRNLSGQTIQSWVSDGTVKDFYLMPGTYQFVETAAPEGYELAAPITFTVQDNGEVIIQGRLTRGDVHI

SEQ ID NO: 80 - protein, 117 aa, Artificial sequence
  KatI cloning sequence
  MISC_FEATURE (1)..(117): KatI cloning sequence
FSMGIDTMSGLSGETGQSGNTTIEEDSTTHVKFSKRDSNGKELAGAMIELRNLSGQTIQSWVSDGTVKDFYLMPGTYQFVETAAPEGYELAAPITFTVQDNGEVQIQGKATRGDVPI

(19) United States
(12) Patent Application Publication
(10) Pub. No.: US 2023/0321260 A1
(43) Pub. Date: Oct. 12, 2023

(54) PEPTIDE TAGS AND BINDING PARTNERS

(71) Applicant: AdaptVac ApS, Copenhagen N (DK)

(72) Inventors: Cyrielle Elyette FOUGEROUX, Copenhagen (DK); Adam Frederik Sander BERTELSEN, Dragør (DK); Susan THRANE, København N (DK)

(21) Appl. No.: 17/917,214

(22) PCT Filed: May 7, 2021
(86) PCT No.: PCT/EP2021/062113

§ 371 Date: Oct. 5, 2022

(30) Foreign Application Priority Data
May 7, 2020 (EP) 20173377.1

(51) Int. Cl. A61K 47/64; C07K 14/315; G01N 33/531; A61K 39/215

(52) U.S. Cl. CPC A61K 47/646; A61K 39/215; G01N 33/531; C07K 14/315

(57) ABSTRACT

The present invention relates to peptide tags and binding partners which are capable of interacting via the spontaneous formation of an isopeptide bond, as well as to associated peptide pairs and methods for designing peptide tags, binding partners and peptide pairs with improved properties.

### TECHNICAL FIELD

The present invention relates to peptide tags and binding partners which are capable of interacting via the spontaneous formation of an isopeptide bond, as well as to associated peptide pairs and methods for designing peptide tags, binding partners and peptide pairs with improved properties.

### SEQUENCE LISTING

The instant application contains a Sequence Listing which has been submitted electronically as a .txt file and is hereby incorporated by reference in its entirety. Said .txt file, created on Jun. 21, 2023, is named ADPT0001PA_corrected_sequence_list_ST25.txt and is 40,578 bytes in size.

### BACKGROUND

The use of peptides and peptide-like molecules as tags for attaching to proteins and other entities is an important tool in molecular biology. Such peptide tags can allow the detection, purification and analysis of a particular protein or entity or can be used for the specific targeting of the tagged protein or entity. They can also be used for generating nanoparticles or virus-like particles, as described in e.g. WO 2016/112921.

Peptide tags, which can be attached to a protein of interest using recombinant DNA methods (e.g. by operably linking the nucleotide sequence encoding the peptide tag with the gene encoding the protein of interest and expressing the protein product), usually have the ability to bind to a binding partner. This binding may allow the detection of the protein of interest if the binding partner is detectable, e.g. is an antibody or is conjugated to a detectable entity, or can allow purification of the protein of interest if the binding partner is, for example, immobilised to a solid support. Alternatively, peptide tags and their binding partners can be used to display molecules of interest, for example antigens, on the surface of virus-like particles (VLPs) or nanoparticles.

Thus, the use of peptide tags which are capable of associating with a binding partner has vast applications and can provide a means for manipulating or analysing a target protein or entity, for generating bispecific molecules that can be used in cancer immunotherapy, for CAR T-cell therapy or for generating e.g. VLP-based vaccines.

Several peptide tag/binding partner systems have been described, which provide high affinity or irreversible binding and which would be useful for the above applications.

In particular, systems where the peptide tag and the binding partner interact via an isopeptide bond are useful. Such pairs have stable or irreversible interactions by the spontaneous formation of isopeptide bonds between the peptide tag and its binding partner.

Isopeptide bonds are amide bonds formed between carboxyl/carboxamide and amino groups, where at least one of the carboxyl or amino groups is outside of the protein main-chain (the backbone of the protein). Such bonds are chemically irreversible under biological conditions and are resistant to most proteases.

### SUMMARY

The invention is as defined in the claims.

The present invention thus uses proteins which are capable or susceptible of forming spontaneous isopeptide bonds, to develop improved peptide tag/binding partner pairs which covalently bind to each other and hence provide improved irreversible interactions.

In this respect, proteins which are capable of spontaneous isopeptide bond formation may be expressed as separate fragments, to give a peptide tag and a binding partner for the peptide tag, where the two fragments are capable of covalently reconstituting the proteins by isopeptide bond formation. This covalent reaction through an isopeptide bond makes the peptide-protein interaction stable under conditions where non-covalent interactions would rapidly dissociate.

As discussed in detail below, the peptide tag preferably comprise one of the residues involved in the isopeptide bond in the original protein and the binding partner preferably comprises the other residue involved in the isopeptide bond in the original protein.

Hence, the coding sequence for the protein may be cleaved to form fragments which encode the peptide tag and binding partner pair.

Herein is provided a method of producing a modified binding partner capable of binding to a peptide tag via the spontaneous formation of an isopeptide bond between one reactive residue comprised within said modified binding partner and another reactive residue comprised within said peptide tag, said method comprising the steps of:
i) Selecting at least a first pair of peptides consisting of a first peptide tag and a first binding partner and a second pair of peptides consisting of a second peptide tag and a second binding partner, wherein for each pair of peptides the peptide tag and the binding partner are capable of or suspected of being capable of binding to each other by spontaneously forming an isopeptide bond;
ii) Identifying the position of the isopeptide bond for the first pair of peptides and/or the second pair of peptides, thereby identifying a first reactive fragment of the first binding partner and/or a second reactive fragment of the second binding partner, and a first residual fragment of the first binding partner and/or a second residual fragment of the second binding partner; wherein the first and/or second reactive fragment comprises the reactive residue involved in the isopeptide bond;
iii) Designing the modified binding partner, wherein the modified binding partner comprises or consists of i) the first reactive fragment, or a homologue thereof having at least 70% homology thereto, and the second residual fragment, or a homologue thereof having at least 70% homology thereto, wherein the first reactive fragment preferably is upstream of the second residual fragment, wherein the modified binding partner does not comprise both reactive residues involved in the formation of the isopeptide bond;
iv) Producing the modified binding partner.

Also provided herein are modified binding partners obtainable by the methods disclosed herein.

Also provided herein is a modified binding partner capable of binding to a peptide tag via the spontaneous formation of an isopeptide bond between one reactive residue comprised within said modified binding partner and another reactive residue comprised within said peptide tag, wherein the modified binding partner does not comprise both reactive residues involved in the formation of the isopeptide bond, and wherein the modified binding partner comprises or consists of a first reactive fragment of a first binding partner comprising one reactive residue capable of interacting with a first peptide tag comprising another reactive residue via the formation of an isopeptide bond between the reactive residues, or a homologue thereof having at least 70% homology thereto, and a second residual fragment of a second binding partner, wherein said second binding partner is capable of interacting with a second peptide tag comprising another reactive residue via the formation of an isopeptide bond between the reactive residues, wherein the second residual fragment does not comprise the reactive residue, or a homologue thereof having at least 70% homology thereto, preferably wherein the first reactive fragment is upstream of the second residual fragment.

Also provided herein is a method of producing a peptide tag capable of binding to a binding partner via the spontaneous formation of an isopeptide bond between one reactive residue comprised within said peptide tag and another reactive residue comprised within said binding partner, preferably wherein said binding partner is a modified binding partner disclosed herein, said method comprising the steps of:
a) Identifying candidate peptide tags having at least 60% similarity to a reference peptide tag, wherein the reference peptide tag is capable of spontaneously forming an isopeptide bond with at least one reference binding partner, preferably wherein the reference peptide tag comprises a reference binding motif;
b) Selecting peptide tags from the candidate peptide tags identified in a), wherein the selected peptide tags comprise at least one reactive residue potentially involved in the formation of the isopeptide bond;
c) Designing and producing the peptide tag from the selected peptide tags, wherein each peptide tag comprises or consists of a fragment of the selected peptide tags spanning from 4 to 24 amino acids upstream to 2 to 22 amino acids downstream of the reactive residue potentially involved in the formation of the isopeptide bond, or a homologue thereof having at least 70% homology thereto, with the proviso that the homologue comprises the reactive residue.

Also provided herein is a peptide tag comprising or consisting of a fragment of a protein comprising at least one reactive residue involved in the formation of an isopeptide bond between said peptide tag and a binding partner, wherein the peptide tag comprises or consists of a fragment of said protein spanning from 4 to 24 amino acids upstream to 2 to 22 amino acids downstream of the reactive residue, or a homologue thereof having at least 70% homology thereto, with the proviso that the homologue comprises the reactive residue, preferably wherein the reactive residue is an asparagine or an aspartate.

Also provided herein is a method of producing a peptide pair comprising or consisting of a modified binding partner and a peptide tag, wherein the modified binding partner is capable of binding to the peptide tag via the spontaneous formation of an isopeptide bond between one reactive residue comprised within said modified binding partner and another reactive residue comprised within said peptide tag, said method comprising the steps of:
i) producing a modified binding partner by the method disclosed herein; and/or
ii) producing a peptide tag by the method disclosed herein.

Also provided herein is a peptide pair comprising or consisting of a peptide tag as defined in any one of the preceding items and a modified binding partner as defined herein.

Also provided herein is a modified binding partner having one or more improved properties compared to a reference binding partner, wherein the one or more improved properties are independently selected from one or more of:
a) increased binding efficacy of the modified binding partner to a peptide tag relative to the binding of the reference binding partner to said peptide tag, wherein said modified binding partner and optionally said reference binding partner are capable of binding to said peptide tag via the spontaneous formation of an isopeptide bond between one reactive residue comprised within said modified binding partner or within said reference binding partner, and another reactive residue comprised within said peptide tag, wherein an increased binding efficacy is at least one of the total binding and the binding rate;
b) increased ability to form a particle displaying a peptide of interest, such as a virus-like particle displaying a peptide of interest such as a virus-like particle, wherein the particle displays a peptide of interest, wherein the particle comprises a particle-forming protein such as a virus-like particle-forming protein fused to the modified binding partner and the peptide of interest fused to the peptide tag, or wherein the particle comprises the particle-forming protein fused to the peptide tag and the peptide of interest fused to the modified binding partner, and wherein the particle is formed by spontaneous formation of the isopeptide bond between the modified binding partner and the peptide tag, when compared to the ability of the reference binding partner to form a particle under similar conditions;
c) increased ability to display a peptide of interest on a particle such as a virus-like particle, wherein the particle comprises a particle-forming protein such as a virus-like particle-forming protein fused to the modified binding partner and the peptide of interest fused to the peptide tag, or wherein the particle comprises the particle-forming protein fused to the peptide tag and the peptide of interest fused to the modified binding partner, and wherein the particle is formed by spontaneous formation of the isopeptide bond between the modified binding partner and the peptide tag, when compared to the ability of the reference binding partner to display the peptide of interest under similar conditions.

Also provided herein is a peptide tag having one or more improved properties compared to a reference peptide tag, wherein the one or more improved properties are independently selected from one or more of:
a) increased binding efficacy of the peptide tag to a reference binding partner relative to the binding of the reference peptide tag to said reference binding partner, wherein said peptide tag and said reference peptide tag are capable of binding to said reference binding partner via the spontaneous formation of an isopeptide bond between one reactive residue comprised within said peptide tag or within said reference peptide tag, and another reactive residue comprised within said reference binding partner, wherein the binding efficacy is increased if at least one of the total binding and the binding rate is increased;
b) increased ability to form a particle such as a virus-like particle, wherein the particle displays a peptide of interest, wherein the particle comprises a particle-forming protein such as a virus-like particle-forming protein fused to the reference binding partner and the peptide of interest fused to the peptide tag, or wherein the particle comprises the particle-forming protein fused to the peptide tag and the peptide of interest fused to the reference binding partner, and wherein the particle is formed by spontaneous formation of the isopeptide bond between the reference binding partner and the peptide tag, when compared to the ability of the reference peptide tag to form a particle under similar conditions;
c) increased ability to display a compound of interest such as a peptide on a particle such as a virus-like particle, wherein the particle comprises a particle-forming protein such as a virus-like particle-forming protein fused to the peptide tag and the compound of interest fused to the binding partner, or wherein the particle comprises the particle-forming protein fused to the binding partner and the compound of interest fused to the peptide tag, and wherein the particle is formed by spontaneous formation of the isopeptide bond between the binding partner and the peptide tag, when compared to the ability of the reference peptide tag to display the compound of interest under similar conditions.

Also provided herein is a peptide pair comprising or consisting of a peptide tag and a binding partner, wherein the peptide pair has one or more improved properties compared to a reference peptide pair comprising a reference peptide tag and a reference binding partner,
wherein the binding partner is capable of binding to the peptide tag via the spontaneous formation of an isopeptide bond between one reactive residue comprised within said modified binding partner, and another reactive residue comprised within said peptide tag;
wherein the reference binding partner is capable of binding to the reference peptide tag via the spontaneous formation of an isopeptide bond between one reactive residue comprised within said reference binding partner, and another reactive residue comprised within said reference peptide tag;
wherein the one or more improved properties are independently selected from one or more of:
   a) Increased binding efficacy of the binding partner to the peptide tag relative to the binding of the reference binding partner to the reference peptide tag, wherein the binding efficacy is increased if at least one of the total binding and the binding rate is increased;
   b) Increased ability to form a particle displaying a peptide of interest, such as a virus-like particle displaying a peptide of interest, wherein the particle comprises a particle-forming protein such as a virus-like particle-forming protein fused to the binding partner and the compound of interest fused to the peptide tag, or wherein the particle comprises the virus-like particle-forming protein fused to the peptide tag and the compound of interest fused to the binding partner, and wherein the particle is formed by spontaneous formation of the isopeptide bond between the binding partner and the peptide tag, when compared to the ability of the reference peptide pair to form a particle under similar conditions; and/or
   c) increased ability to display a compound of interest such as a peptide on a particle such as a virus-like particle, wherein the particle comprises a particle-forming protein such as a virus-like particle-forming protein fused to the binding partner and the compound of interest fused to the peptide tag, or wherein the particle comprises the particle-forming protein fused to the peptide tag and the compound of interest fused to the binding partner, and wherein the particle is formed by spontaneous formation of the isopeptide bond between the binding partner and the peptide tag, when compared to the ability of the reference peptide pair to display the peptide of interest under similar conditions.

Also disclosed herein are polynucleotides encoding the modified binding partners and/or the peptide tags disclosed herein.

Also provided is a vector comprising a polynucleotide as described herein.

Also provided is a host cell expressing the modified binding partner disclosed herein and/or the peptide tag disclosed herein.

Also provided is a composition comprising:
i) A protein fused to a modified binding partner as described herein, and a compound of interest such as a peptide, for example an antigen, fused to a peptide tag as described herein; or
ii) A protein fused to a peptide tag as described herein, and a compound of interest such as a peptide, for example an antigen, fused to a modified binding partner as described herein,
wherein the modified binding partner and the peptide tag are capable of interacting by the spontaneous formation of an isopeptide bond, and wherein the compound of interest and the protein are linked via an isopeptide bond between the modified binding partner and the peptide tag.

Also provided is a method of manufacturing a pharmaceutical composition as disclosed herein, comprising the steps of:
i) obtaining a first polypeptide comprising or consisting of a modified binding partner as disclosed herein fused to a protein; and obtaining a second polypeptide comprising or consisting of a peptide tag as disclosed herein fused to a compound of interest; or
   obtaining a first polypeptide comprising or consisting of a peptide tag as disclosed herein fused to a protein and obtaining a second polypeptide comprising or consisting of a modified binding partner as disclosed herein fused to a compound of interest;
      contacting the first polypeptide and the second polypeptide, thereby allowing formation of an isopeptide bond between the peptide tag and the modified binding partner; and generating a pharmaceutical composition as disclosed herein.

### DESCRIPTION OF THE DRAWINGS

FIG. 1 Individual catcher domains (i.e. SdyCatcher (SdyC), SpyCatcher (SpyC), Mooncake and KatI) were mixed with the individual tagged VLPs (i.e. SdyT-Ap205, SpyT-AP205, RumTrunkTag-AP205, RumTrunkD9NTag-Ap205) to get a final concentration of 5 μM for each binding partner. Each of these mixed samples were then incubated at 37° C. for 1 min, 5 min, 10 min, 20 min, 40 min, 1 h, 1 h30 or 3 h. After incubation, individual samples were run on SDS gels with DTT. Percentage of reconstitution was calculated based on densitometric measurement.

FIG. 2A A solution (PBS) containing 10 μM of individual soluble catcher domains (Mooncake, KatI) or catcher-VLP (Mooncake-AP205, KATI-AP205, SpyC-AP205) was mixed in a 1:1 ratio with a solution (PBS) containing 10 μM soluble-tag (RumtrunkD9NTag) (final concentration of each Tag/Catcher binding partner=5 μM). These mixed samples were incubated at 37° C. for 1 min, 5 min, 10 min, 20 min, 40 min, 1 h, 1 h30 or 3 h. After incubation, samples were run on SDS gels with DTT. Percentage of reconstitution was calculated based on densitometric measurement.

FIG. 3 This figure shows one possible embodiment taking advantage of a spontaneous isopeptide bond to display a compound of interest on the surface of a particle, for example a virus-like particle. A particle-forming protein (black lines) able to form a particle, is fused to a peptide tag (black circles). A compound of interest, for example a peptide (dark grey drops) is fused to a binding partner (light grey, half circles). Upon contact, an isopeptide bond forms spontaneously between the peptide tag and the binding partner, which results in the compound of interest being displayed on the surface of the particle.

FIG. 4 Groups of mice (n=6) were immunized prime-boost with an equal dose (6 mcg) of MoonCake-HER2 virus-like particles (VLP) (LCG), SpyCatcher-HER2 VLP (SPY), or PBS respectively (for the PBS group, n=5). Serum was obtained two weeks after each immunization and levels of antigen-specific IgM and IgG (subclasses 1, 2a, 2b and 3) were measured by ELISA. Immunization with MoonCake-HER2 VLP induced significantly higher antigen-specific total Ig compared to SpyCatcher-HER2 VLP.

FIG. 5 Groups of mice were immunized prime-boost with an equal dose (6 mcg) of MoonCake-HER2 VLP or SpyCatcher-HER2 VLP, respectively. Serum was obtained two weeks after each immunization and levels of antigen-specific IgM and IgG (subclasses 1, 2a, 2b and 3) were measured by ELISA. Immunization with MoonCake-HER2 VLP induced significantly higher IgM as well as IgG2a and IgG2b—compared to SpyCatcher-HER2 VLP.

### DETAILED DESCRIPTION

### Definitions

The term “isopeptide bond” as used herein, refers to an amide bond between a carboxyl group and an amino group at least one of which is not derived from a protein main chain or alternatively viewed is not part of the protein backbone. An isopeptide bond may form within a single protein or may occur between two peptides or a peptide and a protein. Thus, an isopeptide may form intramolecularly within a single protein or intermolecularly i.e. between two peptide/protein molecules. Typically, an isopeptide bond may occur intramolecularly between two reactive amino acids: a lysine and an asparagine or aspartate. For the process to occur the two reactive amino acids need to be in dose proximity in a hydrophobic environment often including aromatic residues. Finally, the autocatalytic process may be facilitated by a catalytic aspartate or glutamate residue, which does not themselves take part in the isopeptide bond.

In the case of intermolecular isopeptide bonds, the bond typically occurs between a lysine residue and an asparagine, aspartic acid, glutamine, or glutamic acid residue or the terminal carboxyl group of the protein or peptide chain or may occur between the alpha-amino terminus of the protein or peptide chain and an asparagine, aspartic acid, glutamine or glutamic acid. Each residue of the pair involved in the isopeptide bond is referred to herein as a reactive residue. Thus, an isopeptide bond may form between a lysine residue and an asparagine residue or between a lysine residue and an aspartic acid residue. Particularly, isopeptide bonds can occur between the side chain amine of lysine and carboxamide group of asparagine.

A peptide tag and binding partner pair as discussed herein refer to a binding partner (a peptide/protein) and a peptide tag which binds thereto via an isopeptide bond, preferably a spontaneous isopeptide bond. A peptide tag and binding partner pair will covalently bind to one another via an isopeptide bond and thus preferably the peptide tag comprises one of the reactive residues involved in one isopeptide bond used to design the binding partner and the binding partner comprises the other reactive residue involved in that isopeptide bond. These terms are commonly used in the art; the word “catcher” is sometimes used instead of “binding partner”.

The term “spontaneous” as used herein refers to a bond, in particular an isopeptide bond, which can form in a protein or between peptides or proteins (e.g. between 2 peptides or a peptide and a protein) without any other agent (e.g. an enzyme catalyst) being present and/or without chemical modification of the protein or peptide e.g. without native chemical ligation or chemical coupling. A spontaneous isopeptide bond may therefore form of its own accord in the absence of enzymes or other exogenous substances or without chemical modification. Particularly however, a spontaneous isopeptide or covalent bond may require the presence of a glutamic acid or an aspartic acid residue in the protein or in one of the peptides/proteins involved in the bond to allow formation of the bond.

The term “binding properties” herein refers to the binding properties of a peptide to another peptide, for example to the binding properties of a binding partner to a peptide tag as defined herein, or vice versa. The binding properties most relevant in the context of the present disclosure are the total binding and the binding rate.

The term “total binding” (used herein interchangeably with the term “reconstitution rate”) refers to the percentage of molecules of a given binding partner which form an isopeptide bond with a given peptide tag over time. The total binding can be determined as is known in the art, for example a binding partner and a peptide tag are mixed in equimolar amounts, following which the amount of formed complexes of binding partner and peptide tag resulting from the formation of the isopeptide bond is determined as is known in the art, e.g. at a given time point, and expressed relative to the starting amount of binding partner.

The term “binding rate” refers to the speed or rate of formation of an isopeptide bond between a given binding partner and a given peptide tag, as a function of time.

The term “binding efficacy” is an overall term encompassing both the binding rate and the total binding.

The term “variant” as used herein refers to a functional variant of a parent molecule, such as a variant of a protein, for example a binding partner or a peptide tag, which retains the same function as the parent molecule. A variant binding partner thus retains the ability to spontaneously form an isopeptide bond with a peptide tag; a variant of a peptide tag thus retains the ability to spontaneously form an isopeptide bond with a binding partner. Throughout the present disclosure, it will be understood that a variant having at least 70% homology or identity to a given sequence may have at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity to said sequence, for example to a binding partner or a peptide tag.

The present inventors have developed a method for improving the properties, in particular the binding properties, of a binding partner to a peptide tag, and vice versa. In short, the inventors have found that one or more properties of such modified binding partners and/or such modified peptide tags are improved compared to the starting binding partner and/or peptide tag. The general principle of how such modified binding partners can be designed is described in e.g. Example 1.

Modified Binding Partner with Improved Properties

### Method of Producing a Modified Binding Partner

The present disclosure provides a method of producing a modified binding partner capable of binding to a peptide tag via the spontaneous formation of an isopeptide bond between one reactive residue comprised within said modified binding partner and another reactive residue comprised within said peptide tag, said method comprising the steps of:
i) Selecting at least a first pair of peptides consisting of a first peptide tag and a first binding partner and a second pair of peptides consisting of a second peptide tag and a second binding partner, wherein for each pair of peptides the peptide tag and the binding partner are capable of or suspected of being capable of binding to each other by spontaneously forming an isopeptide bond;
ii) Identifying the position of the isopeptide bond for the first pair of peptides and/or the second pair of peptides, thereby identifying a first reactive fragment of the first binding partner and/or a second reactive fragment of the second binding partner, and a first residual fragment of the first binding partner and/or a second residual fragment of the second binding partner; wherein the first and/or second reactive fragment comprises the reactive residue involved in the isopeptide bond;
iii) Designing the modified binding partner, wherein the modified binding partner comprises or consists of the first reactive fragment, or a homologue thereof having at least 70% homology thereto, and the second residual fragment, or a homologue thereof having at least 70% homology thereto, wherein the first reactive fragment is upstream of the second residual fragment, wherein the second reactive fragment is upstream of the first residual fragment; and wherein the modified binding partner does not comprise both reactive residues involved in the formation of the isopeptide bond;
iv) Producing the modified binding partner.

In a first step, two pairs of peptides are selected: a first pair of peptides, which consists of a first peptide tag and a first binding partner, and a second pair of peptides, which consists of a second peptide tag and a second binding partner. The peptide tag and the binding partner of a given pair of peptide are capable of (or suspected to be capable of) binding to each other by spontaneous formation of an isopeptide bond between the peptide tag and the binding partner. Such peptide pairs are known in the art, and are further described herein below. The method may be applied to peptides or polypeptides which are suspected of being capable of binding to one another via the formation of a spontaneous isopeptide bond.

For each pair of peptides, the position of the isopeptide bond (or the assumed position of the isopeptide bond) is determined within each pair. Each of the binding partner and the peptide tag comprise one of the reactive residues involved in the isopeptide bond. For many peptide pairs, the position of the isopeptide bond is known and can be retrieved from the literature. In other cases, candidate positions which may be involved in the formation of the isopeptide bond can be determined as is known in the art, for example by sequence mining, querying the sequences of the peptide tag and/or binding partner for known motifs, among others.

Identifying the position of the isopeptide bond amounts to identifying the position of the two residues involved in the isopeptide bond. One of these two residues is present in the binding partner, the other in the peptide tag. Once the position of the residue involved in the isopeptide bond has been determined in the binding partner, a fragment is identified which comprises this residue; this fragment is herein also termed a reactive fragment. The remaining part of the binding partner is termed the residual fragment.

A first reactive fragment is thus identified for the first binding partner, which comprises one residue involved in the first isopeptide bond, while the first peptide tag comprises the other residue; the remaining part of the first binding partner is the first residual fragment. A second reactive fragment is thus identified for the second binding partner, which comprises one residue involved in the second isopeptide bond, while the second peptide tag comprises the other residue; the remaining part of the second binding partner is the second residual fragment. The first isopeptide bond refers to the isopeptide bond between the first binding partner and the first peptide tag; the second isopeptide bond refers to the isopeptide bond between the second binding partner and the second peptide tag.

In a following step of the method, a modified binding partner is designed. This modified binding partner comprises or consists in some embodiments of:
i) The first reactive fragment of the first binding partner, or a homologue thereof having at least 70% homology thereto, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto, and
ii) The second residual fragment of the second binding partner, or a homologue thereof having at least 70% homology thereto, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto,

Preferably wherein the first reactive fragment is upstream of the second residual fragment.

In a final step, the modified binding partner is produced. This is done e.g. as known in the art.

The binding partner which has highest homology or identity with the modified binding partner is preferably used as reference to determine whether the modified binding partner has improved properties, as described herein below. In some embodiments, the modified binding partner has higher homology or identity or similarity to the first binding partner than to the second binding partner, and the first binding partner is used as reference. In other embodiments, the modified binding partner has higher homology to the second binding partner than to the first binding partner, and the second binding partner is used as reference.

In some embodiments, the first or the second binding partner to which the modified binding partner has highest homology, similarity or identity is SEQ ID NO: 1 or a homologue thereof having at least 60% homology thereto, such as at least 65%, such as at least 70%, such as at least 75%, such as at least 80%, such as at least 85%, 93%, such as at least 94%, such as at least 95%, such as at least 96%, such as at least 97%, such as at least 98%, such as at least 99% homology thereto, and the reference binding partner is SEQ ID NO: 1 or said homologue thereof.

In some embodiments, the first or the second binding partner to which the modified binding partner has highest homology, similarity or identity is SEQ ID NO: 3 or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto, and the reference binding partner is SEQ ID NO: 3 or said homologue thereof.

In some embodiments, the first or the second binding partner to which the modified binding partner has highest homology, similarity or identity is SEQ ID NO: 9 or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto, and the reference binding partner is SEQ ID NO: 9 or said homologue thereof.

In some embodiments, the first or the second binding partner to which the modified binding partner has highest homology, similarity or identity is SEQ ID NO: 13 or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto, and the reference binding partner is SEQ ID NO: 13 or said homologue thereof.

In some embodiments, the first or the second binding partner to which the modified binding partner has highest homology, similarity or identity is SEQ ID NO: 15 or a homologue thereof having at least 60% homology thereto, such as at least 65%, such as at least 70%, such as at least 75%, such as at least 80%, such as at least 85%, 93%, such as at least 94%, such as at least 95%, such as at least 96%, such as at least 97%, such as at least 98%, such as at least 99% homology thereto, and the reference binding partner is SEQ ID NO: 15 or said homologue thereof.

In some embodiments, the first or the second binding partner to which the modified binding partner has highest homology, similarity or identity is SEQ ID NO: 17 or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto, and the reference binding partner is SEQ ID NO: 17 or said homologue thereof.

In some embodiments, the first or the second binding partner to which the modified binding partner has highest homology, similarity or identity is SEQ ID NO: 19 or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto, and the reference binding partner is SEQ ID NO: 19 or said homologue thereof.

In some embodiments, the first or the second binding partner to which the modified binding partner has highest homology, similarity or identity is SEQ ID NO: 23 or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto, and the reference binding partner is SEQ ID NO: 23 or said homologue thereof.

In some embodiments, the first or the second binding partner to which the modified binding partner has highest homology, similarity or identity is SEQ ID NO: 25 or a homologue thereof having at least 60% homology thereto, such as at least 65%, such as at least 70%, such as at least 75%, such as at least 80%, such as at least 85%, 93%, such as at least 94%, such as at least 95%, such as at least 96%, such as at least 97%, such as at least 98%, such as at least 99% homology thereto, and the reference binding partner is SEQ ID NO: 25 or said homologue thereof.

In some embodiments, the first or the second binding partner to which the modified binding partner has highest homology, similarity or identity is SEQ ID NO: 27 or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto, and the reference binding partner is SEQ ID NO: 27 or said homologue thereof.

In some embodiments, the first or the second binding partner to which the modified binding partner has highest homology, similarity or identity is SEQ ID NO: 29 or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto, and the reference binding partner is SEQ ID NO: 29 or said homologue thereof.

In some embodiments, the first or the second binding partner to which the modified binding partner has highest homology, similarity or identity is SEQ ID NO: 30 or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto, and the reference binding partner is SEQ ID NO: 30 or said homologue thereof.

In some embodiments, the first or the second binding partner to which the modified binding partner has highest homology, similarity or identity is SEQ ID NO: 31 or a homologue thereof having at least 60% homology thereto, such as at least 65%, such as at least 70%, such as at least 75%, such as at least 80%, such as at least 85%, 93%, such as at least 94%, such as at least 95%, such as at least 96%, such as at least 97%, such as at least 98%, such as at least 99% homology thereto, and the reference binding partner is SEQ ID NO: 31 or said homologue thereof.

In some embodiments, the first or the second binding partner to which the modified binding partner has highest homology, similarity or identity is SEQ ID NO: 37 or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto, and the reference binding partner is SEQ ID NO: 37 or said homologue thereof.

In some embodiments, the first or the second binding partner to which the modified binding partner has highest homology, similarity or identity is SEQ ID NO: 39 or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto, and the reference binding partner is SEQ ID NO: 39 or said homologue thereof.

In some embodiments, the first or the second binding partner to which the modified binding partner has highest homology, similarity or identity is SEQ ID NO: 41 or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto, and the reference binding partner is SEQ ID NO: 41 or said homologue thereof.

### Binding Properties

Once the modified binding partner has been obtained by any of the methods described herein, its binding properties may be determined. Accordingly, in some embodiments the method further comprises a step of determining one or more binding properties of the modified binding partner, wherein said one or more properties are preferably selected from i) the total binding and ii) the binding rate of the modified binding partner to one or more of the first peptide tag, the second peptide tag or a third peptide tag. Determining the corresponding one or more binding properties of the first binding partner and/or of the second binding partner to one or more of the first peptide tag, the second peptide tag or the third peptide tag, allows comparison of the binding properties of the modified binding partner and of the first and/or second binding partner to one or more of the first, second or third peptide tag. An increase in at least one of the total binding and the binding rate indicates an increased binding efficacy, or improved binding properties.

In order to determine the binding properties of a modified binding partner according to the present disclosure, a peptide tag to which the modified binding partner binds or is expected to bind may be required. This peptide tag can be the first peptide tag of the first peptide pair, or the second peptide tag of the second peptide pair, or it may be a third peptide tag, as further described herein below.

Measuring an increase in the binding rate of the modified binding partner to at least one of the first, second and third peptide tag, compared to the binding rate of at least one of the first or second binding partner to the same peptide tag, is indicative of the modified binding partner having increased binding efficacy. Preferably, the binding rate of the modified binding partner to a peptide tag is measured and compared to the binding rate of the first binding partner to the same peptide tag; in some embodiments, the binding rate is the binding rate of the modified or first binding partner to the first peptide tag.

Preferably, said increase in binding rate is at least 5%, such as at least 10%, such as at least 15%, such as at least 20%, such as at least 25%, such as at least 30%, such as at least 40%, such as at least 50%, such as at least 60%, such as at least 70%, such as at least 80%, such as at least 90%, such as at least 100%, or more, compared to the binding rate measured for the first and/or second binding partner to the same peptide tag.

In some embodiments, the binding rate of the modified binding partner to the first peptide tag is measured and compared to the binding rate of the first binding partner to the first peptide tag. In other embodiments, the binding rate of the modified binding partner to the second peptide tag is measured. In other embodiments, the binding rate of the modified binding partner to the third peptide tag is measured. The method may also involve measuring the binding rate of the modified partner to two of the first, second or third peptide tag, or to all of them. Preferably, at least the binding rate of the modified binding partner to the first peptide tag is measured, and compared to the binding rate of the first binding partner to the first peptide tag.

Binding partners Specific binding partners which can be used as starting point in the present methods, i.e. which can be the first and/or the second binding partners, comprise binding partners known to form or suspected to be capable of forming an isopeptide bond with a peptide tag. The specific modified binding partners disclosed herein can also be used as starting point.

Accordingly, the first and/or the second binding partner may be independently selected from SEQ ID NO: 1 (SpyCatcher), SEQ ID NO: 3 (SdyCatcher), SEQ ID NO: 9 (SnoopCatcher), SEQ ID NO: 13, SEQ ID NO: 15, SEQ ID NO: 17, SEQ ID NO: 19, SEQ ID NO: 23, SEQ ID NO: 25, SEQ ID NO: 27, SEQ ID NO: 29, SEQ ID NO: 30, SEQ ID NO: 31 and SEQ ID NO: 33 and homologues thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the first binding partner, i.e. the binding partner that is to be modified or improved using the present methods, is SEQ ID NO: 1 (SpyCatcher), SEQ ID NO: 3 (SdyCatcher), SEQ ID NO: 9 (SnoopCatcher), SEQ ID NO: 13, SEQ ID NO: 15, SEQ ID NO: 17, SEQ ID NO: 19, SEQ ID NO: 23, SEQ ID NO: 25, SEQ ID NO: 27, SEQ ID NO: 29, SEQ ID NO: 30, SEQ ID NO: 31 and SEQ ID NO: 33, or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the second binding partner is SEQ ID NO: 1 (SpyCatcher), SEQ ID NO: 3 (SdyCatcher), SEQ ID NO: 9 (SnoopCatcher), SEQ ID NO: 13, SEQ ID NO: 15, SEQ ID NO: 17, SEQ ID NO: 19, SEQ ID NO: 23, SEQ ID NO: 25, SEQ ID NO: 27, SEQ ID NO: 29, SEQ ID NO: 30, SEQ ID NO: 31 and SEQ ID NO: 33, or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto.

The modified binding partner preferably comprises one of the reactive residues participating in, or suspected of participating in, the formation of an isopeptide bond with a given peptide tag; said peptide tag comprises the other active residue. This means that the modified binding partner preferably comprises the reactive residue of the first binding partner—if homologues of the first binding partner are used, they preferably still comprise this reactive residue.

Preferably, the reactive residue present in the modified binding partner, and originating from the first binding partner, is typically a lysine residue, although it may in some cases be an asparagine residue. Preferably, the reactive residue present in the peptide tag is an asparagine or an aspartate residue. These residues together are forming the isopeptide bond.

Without being bound by theory, a third residue may be involved in the formation of the isopeptide bond. While not directly participating in the bond, this third residue may mediate the formation of the bond. Typically, the third residue is a glutamate residue. The modified binding partner preferably comprises this third residue. In other words, the first reactive fragment of the first binding partner preferably comprises this third residue, which is also present in the modified binding partner.

Generally, the binding partner is larger than its corresponding peptide tag; at least when derived from a protein which naturally forms an isopeptide bond, the binding partner comprises or consists of a larger fragment or portion of that protein compared to the peptide tag. The binding partner may comprise a fragment of the protein which overlaps with a fragment designed to constitute a peptide tag or may comprise a discrete and separate fragment of the protein compared to that of the peptide tag.

In some embodiments, the binding partner (i.e. the first binding partner, the second binding partner and/or the modified binding partner) is at least 20 amino acids in length. Preferably, the binding partner has a length of 5 amino acids or more, such as 10 amino acids or more, such as 15 amino acids or more, such as 20 amino acids or more, such as 25 amino acids, such as 30 amino acids, such as 35 amino acids, such as 40 amino acids, such as 45 amino acids, such as 50 amino acids, such as 60, 70, 80, 90, 100, 125, 150, 175, 200, 225, 250, 275, 300, 325 or 350 amino acids or more. In preferred embodiments, the modified binding partner is at least 20 amino acids in length. Preferably, the binding partner has a length of 5 amino acids or more, such as amino acids or more, such as 15 amino acids or more, such as 20 amino acids or more, such as 25 amino acids, such as 30 amino acids, such as 35 amino acids, such as 40 amino acids, such as 45 amino acids, such as 50 amino acids, such as 60, 70, 80, 90, 100, 125, 150, 175, 200, 225, 250, 275, 300, 325 or 350 amino acids or more.

The present methods can also be used to modify peptides which are suspected to be involved in the formation of an isopeptide bond with a peptide tag. WO 2011/098772 describes in detail how such potential binding partners and peptide tags can be identified. The methods described therein can be used to select a first and/or second peptide pair with the corresponding first and/or second binding partner and peptide tag.

Peptide tags The term “peptide tag” as used herein generally refers to a small peptide fragment which may be designed or derived directly from a protein which naturally forms an intramolecular isopeptide bond. Peptide tags may also be identified by using a known binding partner, for example derived from a protein naturally forming an intramolecular isopeptide bond, to screen a peptide library.

In some embodiments, the first peptide tag, the second peptide tag and/or the third peptide tag used to determine the binding properties of the modified binding partner in the present methods are independently selected from the group consisting of SEQ ID NO: 5 (SpyTag), SEQ ID NO: 7 (SdyTag), SEQ ID NO: 69 (SnoopTag), SEQ ID NO: 46 (RumTag), SEQ ID NO: 47 (RumTrunkD9NTag), SEQ ID NO: 50 (PhoTag), SEQ ID NO: 52 (EntTag), SEQ ID NO: 54 (Rum7Tag), SEQ ID NO: 56 (Rum3Tag), SEQ ID NO: 58 (Rum2Tag), SEQ ID NO: 60 (Rum4Tag), SEQ ID NO: 62 (Rum5Tag), SEQ ID NO: 64 (Rum6Tag), SEQ ID NO: 66 (BacTag), SEQ ID NO: 68 (Bac2Tag), SEQ ID NO: (Bac3Tag), SEQ ID NO: 22 (Bac4Tag), SEQ ID NO: 71 (RumTrunkTag), SEQ ID NO: 46 (RumTag), and SEQ ID NO: 12 (Bac5Tag), or homologues thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto.

A peptide tag may be between 5-50 amino acids in length e.g. from 10, 20, 30, 40 to 50 amino acids in length and may bind covalently via an isopeptide bond to a binding partner as defined herein. Thus, the peptide tag may comprise one reactive residue involved in an isopeptide bond in the isopeptide protein used to design the binding partner (and the binding partner may comprise the other reactive residue involved in that bond), as described herein above.

A peptide tag may be altered, e.g. mutations or alterations may be introduced in any one, any two, or any three of the first, second or third peptide tag.

If a peptide tag is directly designed using protein which naturally forms an intramolecular isopeptide bond, the peptide tag may (i) comprise or consist of a fragment of said protein wherein the fragment is at least 5 amino acids in length or a sequence with at least 50% identity to the fragment e.g. with at least 55, 60, 65, 70, 75, 80, 85, 90, 95, 96, 97, 98 or 99% identity, and (ii) be less than 50 amino acids in length.

The peptide tag may comprise or consist of a fragment of the isopeptide protein which is at least 5 amino acids in length e.g. at least 6, 7, 8, 9, 10, 11, 12, 13, 14, 15, 16, 17, 18 or 19 amino acids in length.

As discussed above, the peptide tag may consist of less than 50 amino acid residues, for example less than 50, 40, 30, 20 or 10 amino acid residues.

Particularly, the peptide tag may consist of 3, 4, 5, 6, 7, 8, 9, 10, 11, 12, 13, 14, 15, 16, 17, 18, 19, 20, 21, 22, 23, 24 or 25 amino acid residues.

As previously discussed, a peptide tag, i.e. a first, second or third peptide tag, should be able to covalently bind to a corresponding binding partner via an isopeptide bond spontaneously. In this respect, the peptide tag preferably comprises one of the reactive amino acid residues involved in the formation of an isopeptide bond in the isopeptide protein. Hence, the peptide tag comprises only one reactive residue from the isopeptide bond and does not comprise both reactive residues involved. Further, if the peptide tag is modified or mutated, the reactive residue in that fragment preferably remains unchanged. This means that when a homologue of a peptide tag is used, the homologue preferably still contains the reactive residue which was originally present in the peptide tag.

Preferably, the reactive residue present in the peptide tag is an asparagine or an aspartate residue, which can form an isopeptide bond with the reactive residue of the binding partner or modified binding partner, as described above.

Without being bound by theory, a third residue may be involved in the formation of the isopeptide bond. While not directly participating in the bond, this third residue may mediate the formation of the bond. Typically, the third residue is a glutamate residue. The modified binding partner preferably comprises this third residue. In other words, the peptide tag, i.e. any of the first, second or third peptide tag, preferably does not comprise this third residue, which is instead present in the modified binding partner.

In some embodiments of the method which involve determining one or more binding properties of the modified binding partner to a peptide tag and comparing them to the binding properties of the first and/or second binding partner to the same peptide tag, the peptide tag is the first peptide tag (of the first binding pair) or the second peptide tag (of the second binding pair) or a third peptide tag, which is different from the first and second peptide tags.

The third peptide tag may be a known peptide tag. It may also be a peptide tag designed in silico. It may also be a peptide present in a peptide library, which can then be screened for new binding pairs—the present methods allow indeed to identify the modified binding partners and candidate peptide tags which have improved binding properties. The third peptide tag may also be designed according to the methods described herein below in the section “Method of producing a peptide tag”.

### Binding Pairs

The methods described herein are particularly useful to identify modified binding partners with improved properties, in particular with improved binding properties in relation to a given peptide tag. Preferably, the present methods are used to improve binding of a binding partner (i.e. the first binding partner) to its peptide tag (i.e. the first peptide tag), thus obtaining improved peptide pairs. The term “improved properties” here refers to any desired property, such as binding rate or total binding, as detailed herein, but also modified specificity toward a given peptide tag—in some cases it may be desirable to decrease the specificity toward a peptide tag, while the specificity toward another peptide tag is unchanged or increased.

Suitable peptide pairs that can be used in the present methods as starting peptide pairs to be improved are for example:
a) the binding partner of SEQ ID NO: 1 (SpyCatcher) and the peptide tag of SEQ ID NO: 5 (SpyTag), or variants thereof having at least 70% homology or identity thereto;
b) the binding partner of SEQ ID NO: 3 (SdyCatcher) and the peptide tag of SEQ ID NO: 7 (SdyTag), or variants thereof having at least 70% homology or identity thereto;
c) the binding partner of SEQ ID NO: 9 (SnoopCatcher) and the peptide tag of SEQ ID NO: 69 (SnoopTag), or variants thereof having at least 70% homology or identity thereto;
d) the binding partner of SEQ ID NO: 39 (MoonCake) and the peptide tag of SEQ ID NO: 47 (RumTrunkD9NTag), or variants thereof having at least 70% homology or identity thereto;
e) the binding partner of SEQ ID NO: 41 (KatI) and the peptide tag of SEQ ID NO: 47 (RumTrunkD9NTag), or variants thereof having at least 70% homology or identity thereto;
f) the binding partner of SEQ ID NO: 39 (MoonCake) and the peptide tag of SEQ ID NO: 46 (RumTag), or variants thereof having at least 70% homology or identity thereto;
g) the binding partner of SEQ ID NO: 41 (KatI) and the peptide tag of SEQ ID NO: 46 (RumTag), or variants thereof having at least 70% homology or identity thereto;
h) the binding partner of SEQ ID NO: 29 (PsCsCatcher) and the peptide tag of SEQ ID NO: 75 (PsCsTag), or variants thereof having at least 70% homology or identity thereto;
wherein a variant of a binding partner or of a peptide tag having at least 70% homology or identity to said binding partner or peptide tag retains the capability of forming an isopeptide bond to the corresponding peptide tag or binding partner, respectively, and has at least 70%, such as at least 71%, %, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity to said binding partner or peptide tag.

### Modified Binding Partner

Also provided herein are modified binding partners obtainable by the methods disclosed herein, and/or modified binding partners with improved properties.

Also provided herein is a modified binding partner capable of binding to a peptide tag via the spontaneous formation of an isopeptide bond between one reactive residue comprised within said modified binding partner and another reactive residue comprised within said peptide tag, wherein the modified binding partner does not comprise both reactive residues involved in the formation of the isopeptide bond, and wherein the modified binding partner comprises or consists of a first reactive fragment of a first binding partner comprising one reactive residue capable of interacting with a first peptide tag comprising another reactive residue via the formation of an isopeptide bond between the reactive residues, or a homologue thereof having at least 70% homology thereto, at least 70% homology thereto, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto, and a second residual fragment of a second binding partner, wherein said second binding partner is capable of interacting with a second peptide tag comprising another reactive residue via the formation of an isopeptide bond between the reactive residues, wherein the second residual fragment does not comprise the reactive residue, or a homologue thereof having at least 70% homology thereto, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto, wherein the first reactive fragment preferably is upstream of the second residual fragment.

The first binding partner and the second binding partner may be as described herein above in the section “Binding partners”. The modified binding partner may be as described herein, in particular in the section “Binding partners”.

Thus in some embodiments, the first and/or the second binding partner, preferably at least the first binding partner, may be independently selected from SEQ ID NO: 1 (SpyCatcher), SEQ ID NO: 3 (SdyCatcher), SEQ ID NO: 9 (SnoopCatcher), SEQ ID NO: 13, SEQ ID NO: 15, SEQ ID NO: 17, SEQ ID NO: 19, SEQ ID NO: 23, SEQ ID NO: 25, SEQ ID NO: 27, SEQ ID NO: 29, SEQ ID NO: 31 and SEQ ID NO: 33 and homologues thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto.

Other relevant binding partners have been described in e.g. Kang et al. 2007,

The modified binding partner preferably comprises one of the reactive residues participating in, or suspected of participating in, the formation of an isopeptide bond with a given peptide tag; said peptide tag comprises the other active residue. This means that the modified binding partner preferably comprises the reactive residue of the first binding partner—if homologues of the first binding partner are used, they preferably still comprise this reactive residue.

Preferably, the reactive residue present in the modified binding partner, and originating from the first binding partner, is typically a lysine residue, although it may in some cases be an asparagine residue. Preferably, the reactive residue present in the peptide tag is an asparagine or an aspartate residue. These residues together are forming the isopeptide bond.

The first peptide tag and the second peptide tag may be as described herein above in the section “Peptide tags”.

In some embodiments, the peptide tag, i.e. the first peptide tag and/or the second peptide tag, and preferably at least the first peptide tag, are independently selected from the group consisting of SEQ ID NO: 5 (SpyTag), SEQ ID NO: 7 (SdyTag), SEQ ID NO: 69 (SnoopTag), SEQ ID NO: 46 (RumTag), SEQ ID NO: 47 (RumTrunkD9NTag), SEQ ID NO: 50 (PhoTag), SEQ ID NO: 52 (EntTag), SEQ ID NO: 54 (Rum7Tag), SEQ ID NO: 56 (Rum3Tag), SEQ ID NO: 58 (Rum2Tag), SEQ ID NO: 60 (Rum4Tag), SEQ ID NO: 62 (Rum5Tag), SEQ ID NO: 64 (Rum6Tag), SEQ ID NO: 66 (BacTag), SEQ ID NO: 68 (Bac2Tag), SEQ ID NO: 35 (Bac3Tag), SEQ ID NO: 22 (Bac4Tag), SEQ ID NO: 71 (RumTrunkTag) and SEQ ID NO: 12 (Bac5Tag), or homologues thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto.

As previously discussed, the peptide tag preferably comprises one of the reactive amino acid residues involved in the formation of an isopeptide bond in the isopeptide protein. Hence, the peptide tag comprises only one reactive residue from the isopeptide bond and does not comprise both reactive residues involved. Further, if the peptide tag is modified or mutated, the reactive residue in that fragment preferably remains unchanged. This means that when a homologue of a peptide tag is used, the homologue preferably still contains the reactive residue which was originally present in the peptide tag.

The present inventors have, using the above methods, designed, produced and tested several modified binding partners having improved properties, as described in the examples below.

These tags can be further improved by mutation or rational design. For example, the tags may be further improved by mutating the reactive residue; for example in peptide tags where the reactive residue is D, mutating this residue to an N improves the properties of the peptide tags—this may further improve the properties of the peptide tags. This may result in increased specificity towards the modified binding partner, sometimes also accompanied by a decrease in specificity towards the first or the second binding partner.

In some embodiments, the modified binding partner comprises or consists of SEQ ID NO: 37 (QueenCatcher), SEQ ID NO: 39 (MoonCake), or SEQ ID NO: 41 (KatI), a fragment or a homologue thereof having at least 60% homology or identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology or identity thereto.

In one embodiment, the modified binding partner comprises or consists of SEQ ID NO: 37 or a fragment or homologue thereof having at least 60% homology or identity thereto, with the proviso that the residue corresponding to residue 31 in SEQ ID NO: 37, which is the active residue, is not modified.

In another embodiment, the modified binding partner comprises or consists of SEQ ID NO: 39 or a fragment or homologue thereof having at least 60% homology or identity thereto, with the proviso that the residue corresponding to residue 31 in SEQ ID NO: 39, which is the active residue, is not modified.

In another embodiment, the modified binding partner comprises or consists of SEQ ID NO: 41 or a fragment or homologue thereof having at least 60% homology or identity thereto, with the proviso that the residue corresponding to residue 31 in SEQ ID NO: 41, which is the active residue, is not modified.

In another embodiment, the modified binding partner comprises or consists of SEQ ID NO: 29 or a fragment or homologue thereof having at least 60% homology or identity thereto, with the proviso that the residue corresponding to residue 8 in SEQ ID NO: 29, which is the active residue, is not modified.

In another embodiment, the binding partner comprises or consists of SEQ ID NO: 9 or a fragment or homologue thereof having at least 60% homology or identity thereto, with the proviso that the residue corresponding to residue 117 in SEQ ID NO: 9, which is the active residue, is not modified.

The modified binding partner set forth in SEQ ID NO: 37 was obtained starting from SEQ ID NO: 3. The reactive residue in SEQ ID NO: 3 is at position 31 of SEQ ID NO: 3, and is retained in the modified binding partner comprising or consisting of SEQ ID NO: 37 or the homologue thereof having at least 70% identity or homology thereto. The first reactive fragment from SEQ ID NO: 3 spans positions 1 to 93 of SEQ ID NO: 3. The modified binding partner of SEQ ID NO: 37 further comprises a fragment (the residual fragment) spanning positions 97 to 116 of SEQ ID NO: 1. The reactive residue in SEQ ID NO: 37 is at position 31.

The modified binding partners set forth in SEQ ID NO: 39 and SEQ ID NO: 41 were both obtained starting from SEQ ID NO: 37. The modified binding partner set forth in SEQ ID NO: 39 and SEQ ID NO: 41 were designed by in silico structure modelling and rational design, introducing mutations in SEQ ID NO: 37. The reactive residue in SEQ ID NO: 39 and SEQ ID NO: 41 is at position 31 of these sequences.

In one embodiment, the modified binding partner comprises or consists of SEQ ID NO: 29 or a fragment or homologue thereof, with the proviso that the residue corresponding to residue 8 in SEQ ID NO: 29, which is the active residue, is not modified.

### Improved Properties

Herein are disclosed methods for modifying binding partners, as described above, as well as modified binding partners. Such modified partners are of particular interest if they display improved properties compared to a reference binding partner. The improved properties may be improved binding properties, as described in detail herein below, or they may be other properties, such as modified specificity towards a given partner, as detailed herein above. For example, the modified binding partners described herein or obtained with the methods described herein may be particularly useful for other applications, such as applications relating to peptide display on a particle, or may have increased stability.

The present disclosure thus also provides a modified binding partner having one or more improved properties compared to a reference binding partner, wherein the one or more improved properties are independently selected from one or more of:
a) increased binding efficacy of the modified binding partner to a peptide tag relative to the binding of the reference binding partner to said peptide tag, wherein said modified binding partner and optionally said reference binding partner are capable of binding to said peptide tag via the spontaneous formation of an isopeptide bond between one reactive residue comprised within said modified binding partner or within said reference binding partner, and another reactive residue comprised within said peptide tag, wherein an increased binding efficacy is at least one of the total binding and the binding rate;
b) increased ability to form a particle displaying a peptide of interest, such as a virus-like particle displaying a peptide of interest, wherein the particle comprises a particle-forming protein such as a virus-like particle-forming protein fused to the modified binding partner and the peptide of interest fused to the peptide tag, or wherein the particle comprises the particle-forming protein fused to the peptide tag and the peptide of interest fused to the modified binding partner, and wherein the particle is formed by spontaneous formation of the isopeptide bond between the modified binding partner and the peptide tag, when compared to the ability of the reference binding partner to form a particle under similar conditions;
c) increased ability to display a peptide of interest on a particle such as a virus-like particle, wherein the particle comprises a particle-forming protein such as a virus-like particle-forming protein fused to the modified binding partner and the peptide of interest fused to the peptide tag, or wherein the particle comprises the particle-forming protein fused to the peptide tag and the peptide of interest fused to the modified binding partner, and wherein the particle is formed by spontaneous formation of the isopeptide bond between the modified binding partner and the peptide tag, when compared to the ability of the reference binding partner to display the peptide of interest under similar conditions

The term “under similar conditions” refers herein to the formation of the same particle displaying the same peptide of interest, but where the display is obtained by fusion of the reference binding partner instead of by fusion of the modified binding partner.

Typically, the binding properties of the modified binding partner will be improved at least when compared to the binding properties of the first binding partner, i.e. the binding partner which contained one reactive residue which is still present in the modified binding partner. It may also be of interest to compare the properties of the modified binding partner to the properties of the binding partner (first or second) to which it has the highest homology, similarity or identity. In other words, throughout this disclosure, the reference binding partner preferably is the one of the first or second binding partner which has most homology, similarity or identity with the modified binding partner. Thus in preferred embodiments, the modified binding partner has more homology, similarity or identity to the first binding partner than to the second binding partner, and the reference binding partner is the first binding partner; or the modified binding partner has more homology, similarity or identity to the second binding partner than to the first binding partner and the reference binding partner is the second binding partner.

However, it will be understood that the binding properties of the modified binding partner may also or alternatively be improved compared to the binding properties of a third binding partner, as described herein above.

The binding properties of the modified binding partner can be determined in relation to the binding to one or more of the first peptide tag, which is the peptide tag capable of interacting via isopeptide bond formation with the first binding partner, the second peptide tag, which is the peptide tag capable of interacting via isopeptide bond formation with the second binding partner, or the third peptide tag, as described herein above in the section “Peptide tags”.

Improved binding properties have been described herein above, and refer in general to an increased binding efficacy of a modified binding partner to a reference peptide tag, where the reference peptide tag is the first peptide tag, the second peptide tag, or the third peptide tag, compared to the binding efficacy of a reference binding partner to the same peptide tag, where the reference binding partner is the first or second binding partner, or a binding partner capable of interacting with the third peptide tag.

The modified binding partner disclosed herein thus preferably has an increased binding efficacy to a given peptide tag when compared to a reference binding partner binding to the same peptide tag; the increased binding efficacy can be an increased binding rate and/or an increased total binding of the modified binding partner to the peptide tag.

In some embodiments, the binding rate of the modified peptide partner is increased by at least 5%, such as at least 10%, such as at least 15%, such as at least 20%, such as at least 25%, such as at least 30%, such as at least 40%, such as at least 50%, such as at least 60%, such as at least 70%, such as at least 80%, such as at least 90%, such as at least 100%, or more compared to the reference binding partner. Preferably the reference binding partner is the one binding partner (first or second) to which the modified binding partner has the highest homology, similarity or identity, and the binding rate is determined in relation to binding of the binding partners to the first peptide tag.

In some embodiments, the total binding of the modified peptide partner is increased by at least 5%, such as at least 10%, such as at least 15%, such as at least 20%, such as at least 25%, such as at least 30%, such as at least 40%, such as at least 50%, such as at least 60%, such as at least 70%, such as at least 80%, such as at least 90%, such as at least 100%, or more compared to the reference binding partner. Preferably the reference binding partner is the one binding partner (first or second) to which the modified binding partner has the highest homology, similarity or identity, and the total binding is determined in relation to binding of the binding partners to the first peptide tag.

In some embodiments, the binding rate of the modified peptide partner is increased by at least 5%, such as at least 10%, such as at least 15%, such as at least 20%, such as at least 25%, such as at least 30%, such as at least 40%, such as at least 50%, such as at least 60%, such as at least 70%, such as at least 80%, such as at least 90%, such as at least 100%, or more compared to the reference binding partner, and the total binding of the modified peptide partner is increased by at least 5%, such as at least 10%, such as at least 15%, such as at least 20%, such as at least 25%, such as at least 30%, such as at least 40%, such as at least 50%, such as at least 60%, such as at least 70%, such as at least 80%, such as at least 90%, such as at least 100%, or more compared to the reference binding partner. Preferably the reference binding partner is the one binding partner (first or second) to which the modified binding partner has the highest homology, similarity or identity, and the total binding is determined in relation to binding of the binding partners to the first peptide tag.

Another property of the modified binding partner which may be improved compared to a reference binding partner is the ability to form a particle displaying a peptide of interest. Such particles can form via particle-forming proteins, as is known in the art, for example, but not only, virus-like particle-forming proteins. By fusing one member of a peptide pair to such a protein and the other member of the peptide pair to a peptide to be displayed on the particle, particles can be obtained which display the peptide;

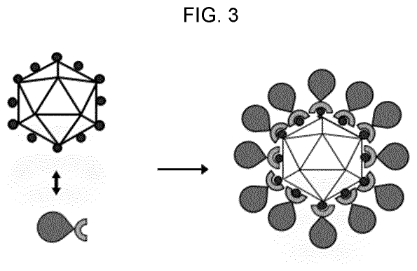

FIG. 3 shows the general principle of one such embodiment. For example, a particle-forming protein is fused to a peptide tag, and the peptide to be displayed is fused to the corresponding binding partner. The spontaneous formation of an isopeptide bond between the peptide tag and its binding partner allows the peptide to be displayed on the surface of the particle. This technique has been used to generate virus-like particles (VLPs) displaying antigenic peptides, e.g. peptides associated with an abnormal physiological response such as a disease, as described in detail in WO 2016/112921. Capsid proteins are examples of suitable particle-forming proteins that can be used to generate such VLPs. For example, AP205, Q13, MS2, HBc, and phage fr, P22, Cowpea mosaic virus (CPMV), Brome mosaic virus (BMV), Cowpea chlorotic mottle virus (CCMV), Bacteriophage lambda Human adenovirus (AdV), Vault particle (PDB: 4V60), can be used. Other suitable proteins are known in the art, for example the proteins listed in Table 1 of Lieknina et al., 2019, can be used.

In some embodiments, the binding partner obtained by the present methods, when used to display a compound of interest such as an antigen, for example in a virus-like particle or in a particle as described below, can lead to an increased immune response when administered to a subject in need thereof, when compared to the immune response obtained from a reference binding partner. The reference binding partner may be as described herein elsewhere. In some embodiments, the reference binding partner is SpyCatcher (SEQ ID NO: 1).

Herein is thus also disclosed a method for inducing an immune response in a subject in need thereof, comprising the administration of a particle such as a virus-like particle comprising the binding partners described herein or obtained by the methods described herein to the subject, where preferably the immune response is increased compared to the immune response obtained after administration of a particle such as a virus-like particle comprising a reference binding partner, such as any of the reference binding partners described herein, in particular SpyCatcher (SEQ ID NO: 1), but otherwise identical.

In some embodiments, the increased immune response is an increased IgM response and/or an increased IgG2 response, such as an increased IgG2a and/or IgG2b. In some embodiments, the increased immune response is an increased IgG1 response. In some embodiments, the increased immune response is an increased IgG3 response. Preferably, at least one of the IgM, IgG2a or IgG2b response is increased.

The method is however generally applicable to generate particles which are not virus-like particles, to display a compound of interest such as a peptide. Other such examples of particle-forming proteins include: small heat-shock protein (HSP) (PDB: 1SHS), Apoferritin (PDB: 1DAT). Pyruvate dehydrogenase multienzyme complex (PDB: 1 EAA), Thermosome (THS) and i301 (designed from the 2-keto-3-deoxy-phosphogluconate (KDPG) aldolase from the Entner-Doudoroff pathway of the hyperthermophilic bacterium Thermotoga maritima).

According to the present methods a protein, which can self-assemble into a nanoparticle, can be genetically modified by fusion of a peptide tag. The assembled nanoparticles (i.e. displaying the reactive peptide tag) can then be coupled to a peptide genetically fused to a binding partner capable of interacting with the peptide tag via an isopeptide bond when contacted with the peptide tag.

The particles thus obtained using the modified binding partner obtained or obtainable by the methods disclosed therein may have improved properties, e.g. in terms of peptide display density (i.e. how many peptide molecules are displayed on the surface of the particle), display homogeneity (i.e. regular and homogenous spacing of the peptide on the surface of the particle), and immunogenicity when displaying antigenic peptides.

The modified binding partner may in other embodiments be used for detection of a compound of interest, in particular a protein of interest, if the modified binding partner is detectable, or it can allow purification of the protein of interest if the binding partner is, for example, immobilised to a solid support.

### Peptide Tags

### Method of Producing a Peptide Tag

The present disclosure also provides a method of producing a peptide tag capable of binding to a binding partner via the spontaneous formation of an isopeptide bond between one reactive residue comprised within said peptide tag and another reactive residue comprised within said binding partner, said method comprising the steps of:
a) Identifying candidate peptide tags having at least 60% similarity to a reference peptide tag, such as at least 65%, such as at least 70%, such as at least 75%, such as at least 80%, such as at least 85%, such as at least 90%, such as at least 95%, such as at least 96%, such as at least 97%, such as at least 98%, such as at least 99% homology thereto, wherein the reference peptide tag is capable of spontaneously forming an isopeptide bond with at least one reference binding partner;
b) Selecting peptide tags from the candidate peptide tags identified in a), wherein the selected peptide tags comprise at least one reactive residue potentially involved in the formation of the isopeptide bond;
c) Designing and producing the peptide tag from the selected peptide tags, wherein each peptide tag comprises or consists of a fragment of the selected peptide tags spanning from 4 to 24 amino acids upstream to 2 to 22 amino acids downstream of the reactive residue involved in the formation of the isopeptide bond, or a homologue thereof having at least 70% homology thereto, such as at least 75%, such as at least 80%, such as at least 85%, such as at least 90%, such as at least 95%, such as at least 96%, such as at least 97%, such as at least 98%, such as at least 99% homology thereto, with the proviso that the homologue comprises the reactive residue.

Preferably, the candidate peptide tag is part of a peptide pair, and the candidates are found based on aligning the peptide pair (which may be a single protein consisting of two domains, i.e. a peptide tag and a binding partner, interacting or suspected of interacting intramolecularly via an isopeptide bond) with known peptide pairs. In some embodiments, the peptide tag, instead of, or in addition to, having at least 60% similarity to a reference peptide tag, comprises a portion of 3, 4, 5, 6, 7, 8 amino acids or more, which comprises the reactive residue and shares at least 60% homology, similarity or identity to a stretch of amino acids, for example 3, 4, 5, 6, 7, 8 amino acids, of the reference peptide tag.

In preferred embodiments, the binding partner is a modified binding partner as described herein, and the method thus allows designing peptide tags that can form an isopeptide bond with a modified binding partner as described herein.

In a first step, candidate peptide tags are identified. This can be done by identifying proteins which are suspected of being isopeptide proteins, i.e. proteins in which two domains interact via the formation of an intramolecular isopeptide bond. Such proteins for example have a fragment or domain, typically a C-terminal fragment or domain, which shares at least 60% similarity, homology or identity to a reference peptide tag which is known to interact with at least one reference binding partner via the spontaneous formation of an isopeptide bond. Thus, starting from a known peptide tag, candidate proteins can be identified from which candidate peptide tags can be identified. Throughout this disclosure, the candidate proteins are known or at least suspected to comprise reactive residues which can form an isopeptide bond, either intramolecular or intermolecular. The candidate peptide tags and candidate binding partners are thus preferably limited to domains of candidate proteins as previously defined.

The candidate peptide tags may be from a library, e.g. a peptide library, which can be screened for candidate peptide tags.

The candidate peptide tags which comprise at least one reactive residue involved or potentially involved in the formation of an isopeptide bond are then selected. Peptide tags are then designed from the selected peptide tags.

The peptide tags designed in the last step comprise or consist of a fragment of the selected peptide tags, which spans from a position upstream of the reactive residue to a position downstream of the reactive residue. The upstream position is located 4 to 24 residues or amino acids upstream of the reactive residue, and the downstream position is located 2 to 22 amino acids downstream of the reactive residue.

In some embodiments, the upstream position is located 4, 5, 6, 7, 8, 9, 10, 11, 12, 13, 14, 15, 16, 17, 18, 19, 20, 21, 22, 23 or 24 residues upstream of the reactive residue, and the downstream position is located 2, 3, 4, 5, 6, 7, 8, 9, 10, 11, 12, 13, 14, 15, 16, 17, 18, 19, 20, 21 or 22 residues downstream of the reactive residue.

The peptide tag may thus in some embodiments have a length between 7 and 47 amino acids, such as between 8 and 46 amino acids, such as between 9 and 45 amino acids, such as between 10 and 44 amino acids, such as between 11 and 43 amino acids, such as between 12 and 42 amino acids, such as between 13 and 41 amino acids, such as between 14 and 40 amino acids, such as between 15 and 39 amino acids, such as between 16 and 38 amino acids, such as between 17 and 37 amino acids, such as between 18 and 36 amino acids, such as between 19 and 35 amino acids, such as between 20 and 34 amino acids, such as between 21 and 33 amino acids, such as between 22 and 32 amino acids, such as between 23 and 31 amino acids, such as between 24 and 30 amino acids, such as between 25 and 29 amino acids, such as between 26 and 28 amino acids, such as 27 amino acids. In some embodiments, the peptide tag has a length of 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45 or 46 amino acids.

Homologues or fragments of the peptide tags thus designed and having at least 70% homology thereto may also be useful, with the proviso that the comprise the reactive residue.

Some known peptide tags comprise a binding motif, herein referred to as reference binding motif or reference sequence motif, which can be used to identify candidate peptide tags. The candidate peptide tags may be peptide tags (or proteins) which comprise a sequence motif, for example a reference sequence motif, within their C-terminal region, for example within 50 amino acids of their C-terminus. If the peptide tag is in the form of a “free” peptide, i.e. an independent peptide, the term “C-terminus” refers to the C-terminal end of that peptide. The peptide tag may however be a part of a protein, as detailed herein above, in which case the term “C-terminus” refers to the C-terminal part of the domain corresponding to the peptide tag in said protein—said C-terminus (of the peptide tag) may thus be located internally within the protein.

The sequence motif may be a motif known to be or suspected to be characteristic of peptide tags capable of forming an isopeptide bond with a binding partner. For example, the sequence motif may comprise or consist of GX₁X₂X₃IVMX₄DX₅ as set forth in SEQ ID NO: 73; GX₁X₂X₃YVMX₄DX₅ as set forth in SEQ ID NO: 74; GX₁X₂X₃FVMX₄DX₅ as set forth in SEQ ID NO: 43; or GX₁X₂X₃VVVMX₄DX₅ as set forth in SEQ ID NO: 44; X₁, X₂, X₃, X₄ and X₅ are independently selected from any amino acid.

The sequence motif may be present in the candidate peptide tags, or it may be present in a protein, for example an isopeptide protein harbouring an intramolecular isopeptide bond, from which candidate peptide tags can then be derived which are fragments of said protein comprising one reactive residue as described herein. The sequence motif may thus be present within 50 amino acids from the C-terminal end of said protein or candidate peptide tag, such as within 45, 40, 35, 30 or 25 amino acids from the C-terminus.

The method may further comprise the step of determining the position of the isopeptide bond (or the assumed position of the isopeptide bond) within the candidate peptide tag (or isopeptide protein). The candidate peptide tags selected for designing peptide tags according to the above method comprise one of the two reactive residues involved in the formation of the isopeptide bond; the other of the two reactive residues being present in the binding partner.

Finally, the designed peptide tag is produced. This is done e.g. as known in the art.

In preferred embodiments, the designed peptide tag is capable of interacting with a modified binding partner as described herein via the spontaneous formation of an isopeptide bond.

In order to determine whether the peptide tag obtained by the present methods has improved properties, its interaction with a reference binding partner is scrutinised. The reference binding partner is preferably a binding partner known to interact with the reference peptide tag. In other words, in order to determine whether the peptide tag obtained by the present methods has improved properties, the properties of said peptide tag with a reference binding partner are preferably compared to the properties of the reference peptide tag with the reference binding partner; binding pairs that can suitably be used as reference are described herein above in the section “Binding pairs”. The reference binding partner may be any of the binding partners described herein, in particular in relation to the first binding partner and the second binding partner. The reference binding partner is in some embodiments a modified binding partner as described herein.

In some embodiments, the reference binding partner is selected from the group consisting of SEQ ID NO: 1 (SpyCatcher), SEQ ID NO: 3 (SdyCatcher), SEQ ID NO: 9 (SnoopCatcher), SEQ ID NO: 13, SEQ ID NO: 15, SEQ ID NO: 17, SEQ ID NO: 19, SEQ ID NO: 23, SEQ ID NO: 25, SEQ ID NO: 27, SEQ ID NO: 29, SEQ ID NO: 30, SEQ ID NO: 31, SEQ ID NO: 37, SEQ ID NO: 39, SEQ ID NO: 41, SEQ ID NO: 71 and SEQ ID NO: 33 and homologues thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto. In some embodiments, the reference binding partner is the binding partner to which the modified binding partner has most homology, similarity or identity.

In some embodiments, the reference peptide tag is selected from the group consisting of SEQ ID NO: 5 (SpyTag), SEQ ID NO: 7 (SdyTag), SEQ ID NO: 69 (SnoopTag), SEQ ID NO: 46 (RumTag), SEQ ID NO: 47 (RumTrunkD9NTag), SEQ ID NO: 50 (PhoTag), SEQ ID NO: 52 (EntTag), SEQ ID NO: 54 (Rum7Tag), SEQ ID NO: 56 (Rum3Tag), SEQ ID NO: 58 (Rum2Tag), SEQ ID NO: 60 (Rum4Tag), SEQ ID NO: 62 (Rum5Tag), SEQ ID NO: 64 (Rum6Tag), SEQ ID NO: 66 (BacTag), SEQ ID NO: 68 (Bac2Tag), SEQ ID NO: 35 (Bac3Tag), SEQ ID NO: 22 (Bac4Tag), SEQ ID NO: 31 and SEQ ID NO: 12 (Bac5Tag), or homologues thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto.

Reference peptide pairs that can be used in the present methods to determine whether the peptide tags designed and produced by the above methods have improved properties are for example:
a) SEQ ID NO: 1 and SEQ ID NO: 5
b) SEQ ID NO: 3 and SEQ ID NO: 7
c) SEQ ID NO: 9 and SEQ ID NO: 69
d) SEQ ID NO: 47 and SEQ ID NO: 39
e) SEQ ID NO: 47 and SEQ ID NO: 41
f) SEQ ID NO: 46 and SEQ ID NO: 39;
g) SEQ ID NO: 46 and SEQ ID NO: 41.

Preferably, the reference peptide pair is selected from SpyTag/SpyCatcher (SEQ ID NO: 5 and SEQ ID NO: 1, respectively) and SdyTag/SdyCatcher (SEQ ID NO: 7 and SEQ ID NO: 3, respectively).

### Binding Properties

Once a peptide tag has been obtained by any of the methods described herein, its binding properties may be determined. Accordingly, in some embodiments the method further comprises a step of determining one or more binding properties of the peptide tag, wherein said one or more properties are preferably selected from i) the total binding and ii) the binding rate of the peptide tag to a reference binding partner, preferably to a modified binding partner. Determining the corresponding one or more binding properties of a reference peptide tag to the same binding partner (i.e. the reference binding partner, preferably a modified binding partner) allows comparison of the binding properties of the peptide tag to said binding partner. An increase in at least one of the total binding and the binding rate indicates an increased binding efficacy, or improved binding properties.

Measuring an increase in the binding rate of the peptide tag to the reference binding partner, compared to the binding rate of a reference peptide tag to said reference binding partner, is indicative of the peptide tag having increased binding efficacy. Preferably, the binding rate of the peptide tag to a reference binding partner is measured and compared to the binding rate of a reference peptide tag to the same binding partner, where the reference peptide tag and reference binding partner (i.e. the reference binding pair) are capable of interacting via the spontaneous formation of an isopeptide bond; in some embodiments, the binding rate is the binding rate of the modified or first binding partner to the first peptide tag. In some embodiments, the reference peptide tag is SpyTag (SEQ ID NO: 5) or SdyTag (SEQ ID NO: 7), and the reference binding partner is SpyCatcher (SEQ ID NO: 1) or SdyCatcher (SEQ ID NO: 3).

Preferably, said increase in binding rate is at least 5%, such as at least 10%, such as at least 15%, such as at least 20%, such as at least 25%, such as at least 30%, such as at least 40%, such as at least 50%, such as at least 60%, such as at least 70%, such as at least 80%, such as at least 90%, such as at least 100%, or more, compared to the binding rate measured for the reference binding pair.

In some embodiments, the binding rate of the peptide tag to the reference binding partner is measured and compared to the binding rate of the reference peptide tag to the reference binding partner.

### Binding Partners

Specific binding partners which can be used as reference binding partners in the present methods, comprise binding partners known to form or suspected to be capable of forming an isopeptide bond with a (reference) peptide tag. The specific modified binding partners disclosed herein can also be used as reference binding partners.

Accordingly, the reference binding partner may be selected from SEQ ID NO: 1 (SpyCatcher), SEQ ID NO: 3 (SdyCatcher), SEQ ID NO: 9 (SnoopCatcher), SEQ ID NO: 13, SEQ ID NO: 15, SEQ ID NO: 17, SEQ ID NO: 19, SEQ ID NO: 23, SEQ ID NO: 25, SEQ ID NO: 27, SEQ ID NO: 29, SEQ ID NO: 30, SEQ ID NO: 31 and SEQ ID NO: 33 and homologues thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto. Preferably, the reference binding partner is SpyCatcher (SEQ ID NO: 1) or SdyCatcher (SEQ ID NO: 3).

In some embodiments, the reference binding partner, to which binding of the peptide tag obtained by the present methods can be measured, is SEQ ID NO: 1 (SpyCatcher), SEQ ID NO: 3 (SdyCatcher), SEQ ID NO: 9 (SnoopCatcher), SEQ ID NO: 13, SEQ ID NO: 15, SEQ ID NO: 17, SEQ ID NO: 19, SEQ ID NO: 23, SEQ ID NO: 25, SEQ ID NO: 27, SEQ ID NO: 29, SEQ ID NO: 30, SEQ ID NO: 31 and SEQ ID NO: 33, or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto.

The reference binding partner preferably comprises one of the reactive residues participating in, or suspected of participating in, the formation of an isopeptide bond with a given reference peptide tag; the reference peptide tag or the peptide tag produced by the above methods comprises the other active residue. Reactive residues have been discussed herein above. The reference binding partner preferably is the one of the first or second binding partner which has most homology, similarity or identity with the modified binding partner. Thus in preferred embodiments, the modified binding partner has more homology, similarity or identity to the first binding partner than to the second binding partner, and the reference binding partner is the first binding partner; or the modified binding partner has more homology, similarity or identity to the second binding partner than to the first binding partner and the reference binding partner is the second binding partner.

In some embodiments, the first or the second binding partner to which the modified binding partner has highest homology, similarity or identity is SEQ ID NO: 1 or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto, and the reference binding partner is SEQ ID NO: 1 or said homologue thereof.

In some embodiments, the first or the second binding partner to which the modified binding partner has highest homology, similarity or identity is SEQ ID NO: 3 or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto, and the reference binding partner is SEQ ID NO: 3 or said homologue thereof.

In some embodiments, the first or the second binding partner to which the modified binding partner has highest homology, similarity or identity is SEQ ID NO: 9 or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto, and the reference binding partner is SEQ ID NO: 9 or said homologue thereof.

In some embodiments, the first or the second binding partner to which the modified binding partner has highest homology, similarity or identity is SEQ ID NO: 13 or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto, and the reference binding partner is SEQ ID NO: 13 or said homologue thereof.

In some embodiments, the first or the second binding partner to which the modified binding partner has highest homology, similarity or identity is SEQ ID NO: 15 or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto, and the reference binding partner is SEQ ID NO: 15 or said homologue thereof.

In some embodiments, the first or the second binding partner to which the modified binding partner has highest homology, similarity or identity is SEQ ID NO: 17 or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto, and the reference binding partner is SEQ ID NO: 17 or said homologue thereof.

In some embodiments, the first or the second binding partner to which the modified binding partner has highest homology, similarity or identity is SEQ ID NO: 19 or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto, and the reference binding partner is SEQ ID NO: 19 or said homologue thereof.

In some embodiments, the first or the second binding partner to which the modified binding partner has highest homology, similarity or identity is SEQ ID NO: 23 or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto, and the reference binding partner is SEQ ID NO: 23 or said homologue thereof.

In some embodiments, the first or the second binding partner to which the modified binding partner has highest homology, similarity or identity is SEQ ID NO: 25 or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto, and the reference binding partner is SEQ ID NO: 25 or said homologue thereof.

In some embodiments, the first or the second binding partner to which the modified binding partner has highest homology, similarity or identity is SEQ ID NO: 27 or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto, and the reference binding partner is SEQ ID NO: 27 or said homologue thereof.

In some embodiments, the first or the second binding partner to which the modified binding partner has highest homology, similarity or identity is SEQ ID NO: 29 or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto, and the reference binding partner is SEQ ID NO: 29 or said homologue thereof.

In some embodiments, the first or the second binding partner to which the modified binding partner has highest homology, similarity or identity is SEQ ID NO: 30 or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto, and the reference binding partner is SEQ ID NO: 30 or said homologue thereof.

In some embodiments, the first or the second binding partner to which the modified binding partner has highest homology, similarity or identity is SEQ ID NO: 31 or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto, and the reference binding partner is SEQ ID NO: 31 or said homologue thereof.

In some embodiments, the first or the second binding partner to which the modified binding partner has highest homology, similarity or identity is SEQ ID NO: 37 or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto, and the reference binding partner is SEQ ID NO: 37 or said homologue thereof.

In some embodiments, the first or the second binding partner to which the modified binding partner has highest homology, similarity or identity is SEQ ID NO: 39 or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto, and the reference binding partner is SEQ ID NO: 39 or said homologue thereof.

In some embodiments, the first or the second binding partner to which the modified binding partner has highest homology, similarity or identity is SEQ ID NO: 41 or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto, and the reference binding partner is SEQ ID NO: 41 or said homologue thereof.

In some embodiments, the first or the second binding partner to which the modified binding partner has highest homology, similarity or identity is SEQ ID NO: 71 or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto, and the reference binding partner is SEQ ID NO: 71 or said homologue thereof.

### Peptide Tags

The term “peptide tag” as used herein generally refers to a small peptide fragment which may be designed or derived directly from a protein which naturally forms an intramolecular isopeptide bond—such proteins are herein termed “isopeptide proteins”. Peptide tags may also be identified by using a known binding partner, for example derived from a protein naturally forming an intramolecular isopeptide bond, to screen a peptide library.

In some embodiments, the reference peptide tag used to determine the binding properties of the peptide tag designed by the methods described herein is selected from the group consisting of SEQ ID NO: 5 (SpyTag), SEQ ID NO: 7 (SdyTag), SEQ ID NO: 69 (SnoopTag), SEQ ID NO: 46 (RumTag), SEQ ID NO: 47 (RumTrunkD9NTag), SEQ ID NO: 50 (PhoTag), SEQ ID NO: 52 (EntTag), SEQ ID NO: 54 (Rum7Tag), SEQ ID NO: 56 (Rum3Tag), SEQ ID NO: 58 (Rum2Tag), SEQ ID NO: 60 (Rum4Tag), SEQ ID NO: 62 (Rum5Tag), SEQ ID NO: 64 (Rum6Tag), SEQ ID NO: 66 (BacTag), SEQ ID NO: 68 (Bac2Tag), SEQ ID NO: 35 (Bac3Tag), SEQ ID NO: 22 (Bac4Tag), SEQ ID NO: 31 and SEQ ID NO: 12 (Bac5Tag), or homologues thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto. Preferably, the reference peptide tag is SpyTag (SEQ ID NO: 5) or SdyTag (SEQ ID NO: 7).

A peptide tag may be between 5-50 amino acids in length e.g. from 10, 20, 30, 40 to 50 amino acids in length and may bind covalently via an isopeptide bond to a binding partner as defined herein. Thus, the peptide tag may comprise one reactive residue involved in an isopeptide bond in the isopeptide protein used to design the binding partner (and the binding partner may comprise the other reactive residue involved in that bond), as described herein above.

If a peptide tag is directly designed using a protein which naturally forms an intramolecular isopeptide bond, the peptide tag may (i) comprise or consist of a fragment of said protein wherein the fragment is at least 5 amino acids in length or a sequence with at least 50% identity to the fragment e.g. with at least 55, 60, 65, 70, 75, 80, 85, 90, 95, 96, 97, 98 or 99% identity, and (ii) be less than 50 amino acids in length. The peptide tag may comprise or consist of a fragment of the isopeptide protein which is at least 5 amino acids in length e.g. at least 6, 7, 8, 9, 10, 11, 12, 13, 14, 15, 16, 17, 18 or 19 amino acids in length.

As discussed above, the peptide tag may consist of less than 50 amino acid residues, for example less than 50, 40, 30, 20 or 10 amino acid residues.

Particularly, the peptide tag may consist of 3, 4, 5, 6, 7, 8, 9, 10, 11, 12, 13, 14, 15, 16, 17, 18, 19, 20, 21, 22, 23, 24 or 25 amino acid residues.

As previously discussed, a peptide tag, i.e. a reference peptide tag or a peptide tag obtained by the methods described herein, should be able to covalently bind to a corresponding binding partner via an isopeptide bond spontaneously. In this respect, the peptide tag preferably comprises one of the reactive amino acid residues involved in the formation of an isopeptide bond in the isopeptide protein. Hence, the peptide tag comprises only one reactive residue from the isopeptide bond and does not comprise both reactive residues involved. Further, if the peptide tag is modified or mutated, the reactive residue in that fragment preferably remains unchanged. This means that when a homologue of a peptide tag is used, the homologue preferably still contains the reactive residue which was originally present in the peptide tag.

### Binding Pairs

The methods described herein are particularly useful to identify peptide tags with improved properties, in particular with improved binding properties to a given binding partner. Preferably, the present methods are used to improve binding of a peptide tag to a reference binding partner (e.g. one of the modified binding partners described herein), thus obtaining improved peptide pairs.

Suitable peptide pairs that can be used in the present methods as starting peptide pairs to be improved are for example:
a) SEQ ID NO: 1 and SEQ ID NO: 5
b) SEQ ID NO: 3 and SEQ ID NO: 7
c) SEQ ID NO: 9 and SEQ ID NO: 69
d) SEQ ID NO: 47 and SEQ ID NO: 39
e) SEQ ID NO: 47 and SEQ ID NO: 41
f) SEQ ID NO: 46 and SEQ ID NO: 39;
g) SEQ ID NO: 46 and SEQ ID NO: 41,

or variants thereof having at least 70% homology or identity thereto.

In some embodiments, the starting peptide pairs are:
a) the binding partner of SEQ ID NO: 1 (SpyCatcher) and the peptide tag of SEQ ID NO: 5 (SpyTag), or variants thereof having at least 70% homology or identity thereto;
b) the binding partner of SEQ ID NO: 3 (SdyCatcher) and the peptide tag of SEQ ID NO: 7 (SdyTag), or variants thereof having at least 70% homology or identity thereto;
c) the binding partner of SEQ ID NO: 9 (SnoopCatcher) and the peptide tag of SEQ ID NO: 69 (SnoopTag), or variants thereof having at least 70% homology or identity thereto;
d) the binding partner of SEQ ID NO: 39 (MoonCake) and the peptide tag of SEQ ID NO: 47 (RumTrunkD9NTag), or variants thereof having at least 70% homology or identity thereto;
e) the binding partner of SEQ ID NO: 41 (KatI) and the peptide tag of SEQ ID NO: 47 (RumTrunkD9NTag), or variants thereof having at least 70% homology or identity thereto;
f) the binding partner of SEQ ID NO: 39 (MoonCake) and the peptide tag of SEQ ID NO: 46 (RumTag), or variants thereof having at least 70% homology or identity thereto;
g) the binding partner of SEQ ID NO: 41 (KatI) and the peptide tag of SEQ ID NO: 46 (RumTag), or variants thereof having at least 70% homology or identity thereto;
h) the binding partner of SEQ ID NO: 29 (PsCsCatcher) and the peptide tag of SEQ ID NO: 75 (PsCsTag), or variants thereof having at least 70% homology or identity thereto;

wherein a variant of a binding partner or of a peptide tag having at least 70% homology or identity to said binding partner or peptide tag retains the capability of forming an isopeptide bond to the corresponding peptide tag or binding partner, respectively, and has at least 70%, such as at least 71%, %, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity to said binding partner or peptide tag.

### Designed Peptide Tags

Also provided herein are peptide tags and binding partners obtainable by the methods disclosed herein, and/or peptide tags with improved properties.

Also provided herein is a peptide tag capable of binding to a reference binding partner, preferably a modified binding partner as described herein, via the spontaneous formation of an isopeptide bond between one reactive residue comprised within said reference binding partner and another reactive residue comprised within said peptide tag, wherein neither the reference binding partner nor the peptide tag (or reference peptide tag) comprise both reactive residues involved in the formation of the isopeptide bond. The reference binding partner may be the modified binding partner described herein above, in particular in the sections “Binding partners” and “Method of producing a modified binding partner”.

Also provided herein is a peptide tag comprising or consisting of a fragment of a protein comprising at least one reactive residue involved in the formation of an isopeptide bond between said peptide tag and a binding partner, wherein the peptide tag comprises or consists of a fragment of said protein spanning from 4 to 24 amino acids upstream to 2 to 22 amino acids downstream of the reactive residue, or a homologue thereof having at least 70% homology thereto, with the proviso that the homologue comprises the reactive residue, preferably wherein the reactive residue is an asparagine or an aspartate.

The peptide tag may comprise a binding motif as described herein above. The sequence motif may be a motif known to be or suspected to be characteristic of peptide tags capable of forming an isopeptide bond with a binding partner. For example, the sequence motif may comprise or consist of GX₁X₂X₃IVMX₄DX₅ as set forth in SEQ ID NO: 73; GX₁X₂X₃YVMX₄DX₅ as set forth in SEQ ID NO: 74; GX₁X₂X₃FVMX₄DX₅ as set forth in SEQ ID NO: 43; or GX₁X₂X₃VVVMX₄DX₅ as set forth in SEQ ID NO: 44; X₁, X₂, X₃, X₄ and X₅ are independently selected from any amino acid.

Useful peptide tags include, but are not limited to, peptide tags selected from the group consisting of: SEQ ID NO: 5 (SpyTag), SEQ ID NO: 7 (SdyTag), SEQ ID NO: 69 (SnoopTag), SEQ ID NO: 46 (RumTag), SEQ ID NO: 47 (RumTrunkD9NTag), SEQ ID NO: 75 (PsCsTag), SEQ ID NO: 50 (PhoTag), SEQ ID NO: 52 (EntTag), SEQ ID NO: 54 (Rum7Tag), SEQ ID NO: 56 (Rum3Tag), SEQ ID NO: 58 (Rum2Tag), SEQ ID NO: 60 (Rum4Tag), SEQ ID NO: 62 (Rum5Tag), SEQ ID NO: 64 (Rum6Tag), SEQ ID NO: 66 (BacTag), SEQ ID NO: 68 (Bac2Tag), SEQ ID NO: 35 (Bac3Tag), SEQ ID NO: 22 (Bac4Tag), SEQ ID NO: 29, SEQ ID NO: 31 and SEQ ID NO: 12 (Bac5Tag) and homologues thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto. In preferred embodiments, the peptide tag is selected from the group consisting of: SEQ ID NO: 46 (RumTag); SEQ ID NO: 71 (RumTrunkTag); SEQ ID NO: 58 (Rum2Tag); SEQ ID NO: 56 (Rum3Tag); SEQ ID NO: 60 (Rum4Tag); SEQ ID NO: 62 (Rum5Tag); SEQ ID NO: 64 (Rum6Tag); SEQ ID NO: 54 (Rum7Tag); SEQ ID NO: 69 (SnoopTag); SEQ ID NO: 66 (BacTag); SEQ ID NO: 68 (Bac2Tag); SEQ ID NO: 35 (Bac3Tag); SEQ ID NO: 22 (Bac4Tag); SEQ ID NO: 12 (Bac5Tag); and SEQ ID NO: 75 (PsCsTag), and homologues thereof having at least 60% homology thereto. In more preferred embodiments, the peptide tag is selected from SEQ ID NO: 46 (RumTag) and SEQ ID NO: 71 (RumTrunkTag) or a homologue thereof having at least 60% homology thereto.

As previously discussed, the peptide tag preferably comprises one of the reactive amino acid residues involved in the formation of an isopeptide bond in the isopeptide protein. Hence, the peptide tag comprises only one reactive residue from the isopeptide bond and does not comprise both reactive residues involved. Further, if the peptide tag is modified or mutated, the reactive residue in that fragment preferably remains unchanged. This means that when a homologue of a peptide tag is used, the homologue preferably still contains the reactive residue which was originally present in the peptide tag.

However, as detailed below, the inventors have found that mutating the reactive residue can advantageously be used to further improve the properties of the designed peptide tag.

The present inventors have, using the above methods, designed, produced and tested several peptide tags having improved properties, as described in the examples below. In some embodiments, the peptide tag has improved binding properties to a binding partner or a modified binding partner which comprises or consists of SEQ ID NO: 37 (QueenCatcher), SEQ ID NO: 39 (MoonCake), SEQ ID NO: 29 (PsCsCatcher), or SEQ ID NO: 41 (KatI), a fragment or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto. Preferably the peptide tag has improved binding properties to a binding partner which comprises or consists of SEQ ID NO: 37 (QueenCatcher), SEQ ID NO: 39 (MoonCake), or SEQ ID NO: 41 (KatI), or a fragment or a homologue thereof.

In one embodiment, the peptide tag has improved binding properties to a modified binding partner which comprises or consists of SEQ ID NO: 37 (QueenCatcher) or a fragment or homologue thereof, with the proviso that the residue corresponding to residue 31 in SEQ ID NO: 37, which is the active residue, is not modified.

In another embodiment, the peptide tag has improved binding properties to a modified binding partner which comprises or consists of SEQ ID NO: 39 (MoonCake) or a fragment or homologue thereof, with the proviso that the residue corresponding to residue 31 in SEQ ID NO: 39, which is the active residue, is not modified. This residue corresponds to residue 35 in SEQ ID NO: 79, which compared to SEQ ID NO: 39 contains 4 residual N-terminal amino acids originating from the cloning procedure.

In another embodiment, the peptide tag has improved binding properties to a modified binding partner which comprises or consists of SEQ ID NO: 41 (KatI) or a fragment or homologue thereof, with the proviso that the residue corresponding to residue 31 in SEQ ID NO: 41, which is the active residue, is not modified. This residue corresponds to residue 35 in SEQ ID NO: 80, which compared to SEQ ID NO: 41 contains 4 residual N-terminal amino acids originating from the cloning procedure.

In another embodiment, the peptide tag has improved binding properties to a binding partner which comprises or consists of SEQ ID NO: 29 (PsCsCatcher) or a fragment or homologue thereof, with the proviso that the residue corresponding to residue 8 in SEQ ID NO: 29, which is the active residue, is not modified.

Herein are thus also provided modified binding partners as described herein above, in particular binding partners as set forth in SEQ ID NO: 39 (MoonCake), or a variant thereof having at least 70% homology or identity thereto; as set forth in SEQ ID NO: 41 (KatI), or a variant thereof having at least 70% homology or identity thereto; as set forth in SEQ ID NO: 37 (QueenCatcher), or a variant thereof having at least 70% homology thereto; as set forth in SEQ ID NO: 9 (SnoopCatcher), or a variant thereof having at least 70% homology or identity thereto; as set forth in SEQ ID NO: 29 (PsCsCatcher), or a variant thereof having at least 70% homology or identity thereto; or as set forth in SEQ ID NO: 37 (QueenCatcher), or a variant thereof having at least 70% homology or identity thereto. Preferably the modified binding partner is as set forth in SEQ ID NO: 39 (MoonCake), or a variant thereof having at least 70% homology or identity thereto; as set forth in SEQ ID NO: 41 (KatI), or a variant thereof having at least 70% homology or identity thereto; or as set forth in SEQ ID NO: 37 (QueenCatcher), or a variant thereof having at least 70% homology thereto.

The modified binding partner set forth in SEQ ID NO: 37 was obtained starting from SEQ ID NO: 3. The reactive residue in SEQ ID NO: 3 is at position 31 of SEQ ID NO: 3, and is retained in the modified binding partner comprising or consisting of SEQ ID NO: 37 or the homologue thereof having at least 70% identity or homology thereto. The first reactive fragment from SEQ ID NO: 3 spans positions 1 to 93 of SEQ ID NO: 3. The modified binding partner of SEQ ID NO: 37 further comprises a fragment (the residual fragment) spanning positions 97 to 116 of SEQ ID NO: 1. The reactive residue in SEQ ID NO: 37 is at position 31.

The modified binding partners set forth in SEQ ID NO: 39 and SEQ ID NO: 41 were both obtained starting from SEQ ID NO: 37. The modified binding partner set forth in SEQ ID NO: 39 and SEQ ID NO: 41 were designed by in silico structure modelling and rational design, introducing mutations in SEQ ID NO: 37. The reactive residue in SEQ ID NO: 39 and SEQ ID NO: 41 is at position 31 of these sequences.

The binding partner as set forth in SEQ ID NO: 39 (MoonCake), or the variant thereof having at least 70% homology or identity thereto, can spontaneously form an isopeptide bond with a peptide tag selected from SEQ ID NO: 47 (RumTrunkD9NTag), SEQ ID NO: 54 (Rum7Tag), SEQ ID NO: 56 (Rum3Tag), SEQ ID NO: 58 (Rum2Tag), SEQ ID NO: 60 (Rum4Tag), SEQ ID NO: 62 (Rum5Tag), SEQ ID NO: 64 (Rum6Tag), SEQ ID NO: 71 (RumTrunkTag), SEQ ID NO: 5 (SpyTag), SEQ ID NO: 7 (SdyTag), SEQ ID NO: 46 (RumTag), SEQ ID NO: 66 (BacTag); SEQ ID NO: 68 (Bac2Tag), SEQ ID NO: 35 (Bac3Tag), SEQ ID NO: 22 (Bac4Tag) and SEQ ID NO: 12 (Bac5Tag); or variants thereof having at least 70% homology or identity thereto. In a preferred embodiment, the binding partner as set forth in SEQ ID NO: 39 (MoonCake), or the variant thereof having at least 70% homology or identity thereto, can spontaneously form an isopeptide bond with the peptide tag as set forth in SEQ ID NO: 47 (RumTrunkD9NTag) or a variant thereof having at least 70% homology thereto. In another embodiment, the binding partner as set forth in SEQ ID NO: 39 (MoonCake), or the variant thereof having at least 70% homology or identity thereto, can spontaneously form an isopeptide bond with the peptide tag as set forth in SEQ ID NO: 46 (RumTag) or a variant thereof having at least 70% homology thereto.

The binding partner as set forth in SEQ ID NO: 41 (KatI), or the variant thereof having at least 70% homology or identity thereto, can spontaneously form an isopeptide bond with a peptide tag selected from SEQ ID NO: 47 (RumTrunkD9NTag), SEQ ID NO: 54 (Rum7Tag), SEQ ID NO: 56 (Rum3Tag), SEQ ID NO: 58 (Rum2Tag), SEQ ID NO: 60 (Rum4Tag), SEQ ID NO: 62 (Rum5Tag), SEQ ID NO: 64 (Rum6Tag), SEQ ID NO: 71 (RumTrunkTag), SEQ ID NO: 5 (SpyTag), SEQ ID NO: 7 (SdyTag), SEQ ID NO: 46 (RumTag), SEQ ID NO: 66 (BacTag); SEQ ID NO: 68 (Bac2Tag), SEQ ID NO: 35

(Bac3Tag), SEQ ID NO: 22 (Bac4Tag) and SEQ ID NO: 12 (Bac5Tag); or variants thereof having at least 70% homology or identity thereto. In a preferred embodiment, the binding partner as set forth in SEQ ID NO: 41 (KatI) or the variant thereof having at least 70% homology or identity thereto, can spontaneously form an isopeptide bond with the peptide tag as set forth in SEQ ID NO: 47 (RumTrunkD9NTag) or a variant thereof having at least 70% homology thereto. In another embodiment, the binding partner as set forth in SEQ ID NO: 41 (KatI), or the variant thereof having at least 70% homology or identity thereto, can spontaneously form an isopeptide bond with the peptide tag as set forth in SEQ ID NO: 46 (RumTag) or a variant thereof having at least 70% homology thereto.

The binding partner as set forth in SEQ ID NO: 9 (SnoopCatcher), or the variant thereof having at least 70% homology or identity thereto, can spontaneously form an isopeptide bond with the peptide tag as set forth in SEQ ID NO: 69 (SnoopTag), or a variant thereof having at least 70% homology thereto.

The binding partner as set forth in SEQ ID NO: 37 (QueenCatcher), or the variant thereof having at least 70% homology or identity thereto, can spontaneously form an isopeptide bond with a peptide tag selected from SEQ ID NO: 5 (SpyTag), SEQ ID NO: 7 (SdyTag), SEQ ID NO: 46 (RumTag), SEQ ID NO: 47 (RumTrunkD9NTag), SEQ ID NO: 54 (Rum7Tag), SEQ ID NO: 56 (Rum3Tag), SEQ ID NO: 58 (Rum2Tag), SEQ ID NO: 60 (Rum4Tag), SEQ ID NO: 62 (Rum5Tag), SEQ ID NO: 64 (Rum6Tag), SEQ ID NO: 71 (RumTrunkTag), SEQ ID NO: 66 (BacTag); SEQ ID NO: 68 (Bac2Tag), SEQ ID NO: 35 (Bac3Tag), SEQ ID NO: 22 (Bac4Tag), SEQ ID NO: 12 (Bac5Tag) and SEQ ID NO: 76 (Clib9) or variants thereof having at least 70% homology or identity thereto. In a preferred embodiment, the binding partner as set forth in SEQ ID NO: 37 (QueenCatcher) or the variant thereof having at least 70% homology or identity thereto, can spontaneously form an isopeptide bond with the peptide tag as set forth in SEQ ID NO: 47 (RumTrunkD9NTag) or a variant thereof having at least 70% homology thereto. In another embodiment, the binding partner as set forth in SEQ ID NO: 37 (QueenCatcher), or the variant thereof having at least 70% homology or identity thereto, can spontaneously form an isopeptide bond with the peptide tag as set forth in SEQ ID NO: 46 (RumTag) or a variant thereof having at least 70% homology thereto.

The binding partner as set forth in SEQ ID NO: 29 (PsCsCatcher) or the variant thereof having at least 70% homology or identity thereto, can spontaneously form an isopeptide bond with the peptide tag as set forth in SEQ ID NO: 75 (PsCsTag), or a variant thereof having at least 70% homology thereto.

### Method for Improving the Properties of a Peptide Tag

Any of the tags obtained by the present methods can be further improved by mutation or rational design. For example, the tags may be further improved by mutating the reactive residue D to an N.

Accordingly, also provided herein is a method for improving the properties of a peptide tag, in particular a peptide tag obtained by the methods disclosed herein. In some embodiments, the peptide tag used as a starting point comprises a reactive residue, which reactive residue is still present in the peptide tag obtained by the methods described above. Mutating this reactive residue may further improve any of the properties of the peptide tag, as detailed herein below. In some embodiments, the methods of designing a peptide tag described herein thus further comprise a step of mutating the reactive residue of the designed peptide tag. In some embodiments, the reactive residue is a D and is mutated to an N.

Thus also provided herein is a method for improving the properties of a peptide tag comprising a reactive residue as described herein above, in particular any of the peptide tags described herein, by mutating at least the reactive residue.

### Improved Properties

The peptide tags described herein are of particular interest if they display improved properties compared to a reference peptide tag, or if they display improved binding to a given binding partner compared to a reference peptide tag. The improved properties may be improved binding properties, as described in detail herein above and below, or they may be other properties. For example, the peptide tags described herein or obtained with the methods described herein may be particularly useful for other applications, such as applications relating to peptide display on a particle.

The present disclosure thus also provides peptide tags having one or more improved properties compared to a reference peptide tag, wherein the one or more improved properties are independently selected from one or more of:
a) increased binding efficacy of the peptide tag to a reference binding partner, in particular a modified binding partner, relative to the binding of the reference peptide tag to said reference binding partner, wherein said peptide tag and optionally said reference peptide tag are capable of binding to said reference binding partner via the spontaneous formation of an isopeptide bond between one reactive residue comprised within said reference binding partner, and another reactive residue comprised within said peptide tag, wherein an increased binding efficacy is at least one of the total binding and the binding rate;
b) increased ability to form a particle displaying a peptide of interest, such as a virus-like particle displaying a peptide of interest, wherein the particle comprises a particle-forming protein such as a virus-like particle-forming protein fused to the reference binding partner and the peptide of interest fused to the peptide tag, or wherein the particle comprises the particle-forming protein fused to the peptide tag and the peptide of interest fused to the reference binding partner, and wherein the particle is formed by spontaneous formation of the isopeptide bond between the reference binding partner and the peptide tag, when compared to the ability of the reference peptide tag to form a particle under similar conditions;
c) increased ability to display a peptide of interest on a particle such as a virus-like particle, wherein the particle comprises a particle-forming protein such as a virus-like particle-forming protein fused to the peptide tag, or wherein the particle comprises the particle-forming protein fused to the peptide tag and the peptide of interest fused to the reference binding partner, and wherein the particle is formed by spontaneous formation of the isopeptide bond between the reference binding partner and the peptide tag, when compared to the ability of the reference peptide tag to display the peptide of interest under similar conditions.

The binding properties of the peptide tag can be determined in relation to the binding to one or more reference binding partner. The reference peptide tag can be as described herein above. In some embodiments, the peptide tag is obtained by further modification of a peptide tag obtained by the methods disclosed herein, e.g. by mutation of the reactive residue, in which case it may be advantageous to compare its properties to the peptide tag which still comprises the original reactive residue. The binding partner which has highest homology or identity with the modified binding partner is preferably used as reference to determine whether the modified binding partner has improved properties, as described herein below. In some embodiments, the modified binding partner has higher homology or identity or similarity to the first binding partner than to the second binding partner, and the first binding partner is used as reference. In other embodiments, the modified binding partner has higher homology to the second binding partner than to the first binding partner, and the second binding partner is used as reference.

Improved binding properties have been described herein above, and refer in general to an increased binding efficacy between a binding partner and a peptide tag.

The peptide tag disclosed herein thus preferably has an increased binding efficacy to a given binding partner when compared to a reference peptide tag binding to the same binding partner; the increased binding efficacy can be an increased binding rate and/or an increased total binding.

In some embodiments, the binding rate of the peptide tag is increased by at least 5%, such as at least 10%, such as at least 15%, such as at least 20%, such as at least 25%, such as at least 30%, such as at least 40%, such as at least 50%, such as at least 60%, such as at least 70%, such as at least 80%, such as at least 90%, such as at least 100%, or more compared to the reference peptide tag.

In some embodiments, the total binding of the peptide tag is increased by at least 5%, such as at least 10%, such as at least 15%, such as at least 20%, such as at least 25%, such as at least 30%, such as at least 40%, such as at least 50%, such as at least 60%, such as at least 70%, such as at least 80%, such as at least 90%, such as at least 100%, or more compared to the reference peptide tag.

In some embodiments, the binding rate is increased by at least 5%, such as at least 10%, such as at least 15%, such as at least 20%, such as at least 25%, such as at least 30%, such as at least 40%, such as at least 50%, such as at least 60%, such as at least 70%, such as at least 80%, such as at least 90%, such as at least 100%, or more compared to the reference peptide tag, and the total binding of the peptide tag is increased by at least 5%, such as at least 10%, such as at least 15%, such as at least 20%, such as at least 25%, such as at least 30%, such as at least 40%, such as at least 50%, such as at least 60%, such as at least 70%, such as at least 80%, such as at least 90%, such as at least 100%, or more compared to the reference peptide tag.

Another property of the peptide tag which may be improved compared to a reference peptide tag is the ability to form a particle displaying a peptide of interest. Such particles can form via particle-forming proteins, as is known in the art, for example, but not only, virus-like particle-forming proteins, and advantage can be taken of binding pairs comprising a peptide tag obtained by the present methods, as described herein above for the modified binding partners in the section “Improved properties”.

In some embodiments, the peptide tag obtained by the present methods, when used to display a compound of interest such as an antigen, for example in a virus-like particle or in a particle as described below, can lead to an increased immune response when administered to a subject in need thereof, compared to the immune response obtained from a particle in which a reference peptide tag is used.

Herein is thus also disclosed a method for inducing an immune response in a subject in need thereof, comprising the administration of a particle such as a virus-like particle comprising the peptide tags described herein or obtained by the methods described herein to the subject, where preferably the immune response is increased compared to the immune response obtained after administration of a particle such as a virus-like particle comprising a reference peptide tag, such as any of the reference peptide tags described herein, but otherwise identical.

In some embodiments, the increased immune response is an increased IgM response and/or an increased IgG2 response, such as an increased IgG2a and/or IgG2b. In some embodiments, the increased immune response is an increased IgG1 response. In some embodiments, the increased immune response is an increased IgG3 response. Preferably, at least one of the IgM, IgG2a or IgG2b response is increased.

In some embodiments, the reference peptide tag is SpyTag (SEQ ID NO: 5) or SdyTag (SEQ ID NO: 7), and the improved binding property is in relation to binding to the corresponding binding partner, i.e. SpyCatcher (SEQ ID NO: 1) or SdyCatcher (SEQ ID NO: 3), respectively.

The particles thus obtained using the peptide tags obtained or obtainable by the methods disclosed therein may have improved properties, e.g. in terms of peptide display density (i.e. how many peptide molecules are displayed on the surface of the particle), display homogeneity (i.e. regular and homogenous spacing of the peptide on the surface of the particle), and immunogenicity when displaying antigenic peptides.

### Peptide Pairs

Also provided herein is a method of producing a peptide pair comprising or consisting of a modified binding partner and a peptide tag, wherein the modified binding partner is capable of binding to the peptide tag via the spontaneous formation of an isopeptide bond between one reactive residue comprised within said modified binding partner and another reactive residue comprised within said peptide tag,
said method comprising the steps of:
i) producing a modified binding partner by any of the methods described herein; and/or
ii) producing a peptide tag by any of the methods described herein.

The following peptide pairs consisting of a binding partner and a peptide tag which are capable of interacting via the spontaneous formation of an isopeptide bond can thus be obtained:
a known binding partner with a peptide tag designed and obtained by any of the above methods;
a modified binding partner designed and obtained by any of the above methods with a known peptide tag;
a modified binding partner designed and obtained by any of the above methods with a peptide tag designed and obtained by any of the above methods.
The binding partner and peptide tag may be any of the binding partners and peptide tags described herein.

The present methods may thus be used to identify peptide pairs, which preferably have improved properties compared to known peptide pairs. In particular, the properties of the peptide pairs obtained by the present methods may be improved compared to the properties of a peptide pair comprising the same binding partner (in the case of a pair comprising a known binding partner) or, in the case of a pair comprising a modified binding partner, to the properties of a peptide pair comprising the binding partner used as a starting point (the first binding partner) and the same peptide tag.

Accordingly, also provided herein is a peptide pair comprising or consisting of a peptide tag and a binding partner, wherein the peptide pair has one or more improved properties compared to a reference peptide pair comprising a reference peptide tag and a reference binding partner,
wherein the binding partner is capable of binding to the peptide tag via the spontaneous formation of an isopeptide bond between one reactive residue comprised within said modified binding partner, and another reactive residue comprised within said peptide tag;
wherein the reference binding partner is capable of binding to the reference peptide tag via the spontaneous formation of an isopeptide bond between one reactive residue comprised within said reference binding partner, and another reactive residue comprised within said reference peptide tag;
   wherein the one or more improved properties are independently selected from one or more of:
   a) increased binding efficacy of the binding partner to the peptide tag relative to the binding of the reference binding partner to the reference peptide tag, wherein the binding efficacy is increased if at least one of the total binding and the binding rate is increased;
   b) increased ability to form a particle displaying a peptide of interest, such as a virus-like particle displaying a peptide of interest, wherein the particle comprises a particle-forming protein such as a virus-like particle-forming protein fused to the binding partner and the peptide of interest fused to the peptide tag, or wherein the particle comprises the virus protein fused to the peptide tag and the peptide of interest fused to the binding partner, and wherein the particle is formed by spontaneous formation of the isopeptide bond between the binding partner and the peptide tag, when compared to the ability of the reference peptide pair to form a particle under similar conditions; and
   c) increased ability to display a peptide of interest on a particle such as a virus-like particle, wherein the particle comprises a particle-forming protein such as a virus-like particle-forming protein fused to the binding partner and the peptide of interest fused to the peptide tag, or wherein the particle comprises the particle-forming protein fused to the peptide tag and the peptide of interest fused to the binding partner, and wherein the particle is formed by spontaneous formation of the isopeptide bond between the binding partner and the peptide tag, when compared to the ability of the reference peptide pair to display the peptide of interest under similar conditions.

In some embodiments, the binding partner is a modified binding partner as described above. The binding partner which has highest homology or identity with the modified binding partner is preferably used as reference to determine whether the modified binding partner has improved properties, as described herein below. In some embodiments, the modified binding partner has higher homology or identity or similarity to the first binding partner than to the second binding partner, and the first binding partner is used as reference. In other embodiments, the modified binding partner has higher homology to the second binding partner than to the first binding partner, and the second binding partner is used as reference.

In some embodiments where the modified binding partner comprises or consists of a first reactive fragment of a first binding partner and a second residual fragment of a second binding partner as defined herein above, the reference binding partner preferably is the second binding partner.

In some embodiments where the modified binding partner comprises or consists of a second reactive fragment of a second binding partner and a first residual fragment of a first binding partner as defined herein above, the reference binding partner preferably is the first binding partner.

The reference binding partner preferably is the one of the first or second binding partner which has most homology, similarity or identity with the modified binding partner. Thus in preferred embodiments, the modified binding partner has more homology, similarity or identity to the first binding partner than to the second binding partner, and the reference binding partner is the first binding partner; or the modified binding partner has more homology, similarity or identity to the second binding partner than to the first binding partner and the reference binding partner is the second binding partner.

In some embodiments, the peptide pair is selected from:
a) SEQ ID NO: 9 and SEQ ID NO: 69
b) SEQ ID NO: 47 and SEQ ID NO: 39;
c) SEQ ID NO: 47 and SEQ ID NO: 41;
d) SEQ ID NO: 46 and SEQ ID NO: 39; and
e) SEQ ID NO: 46 and SEQ ID NO: 41.

In some embodiments, the peptide pair comprises or consists of:
a) the binding partner of SEQ ID NO: 39 (MoonCake) and a peptide tag selected from SEQ ID NO: 47 (RumTrunkD9NTag), SEQ ID NO: 54 (Rum7Tag), SEQ ID NO: 56 (Rum3Tag), SEQ ID NO: 58 (Rum2Tag), SEQ ID NO: 60 (Rum4Tag), SEQ ID NO: 62 (Rum5Tag), SEQ ID NO: 64 (Rum6Tag), SEQ ID NO: 71 (RumTrunkTag), SEQ ID NO: 5 (SpyTag), SEQ ID NO: 7 (SdyTag), SEQ ID NO: 46 (RumTag), SEQ ID NO: 66 (BacTag); SEQ ID NO: 68 (Bac2Tag), SEQ ID NO: 35 (Bac3Tag), SEQ ID NO: 22 (Bac4Tag) and SEQ ID NO: 12 (Bac5Tag); or variants thereof having at least 70% homology or identity thereto;
b) the binding partner of SEQ ID NO: 41 (KatI) and a peptide tag selected from SEQ ID NO: 47 (RumTrunkD9NTag), SEQ ID NO: 54 (Rum7Tag), SEQ ID NO: 56 (Rum3Tag), SEQ ID NO: 58 (Rum2Tag), SEQ ID NO: 60 (Rum4Tag), SEQ ID NO: 62 (Rum5Tag), SEQ ID NO: 64 (Rum6Tag), SEQ ID NO: 71 (RumTrunkTag), SEQ ID NO: 5 (SpyTag), SEQ ID NO: 7 (SdyTag), SEQ ID NO: 46 (RumTag), SEQ ID NO: 66 (BacTag); SEQ ID NO: 68 (Bac2Tag), SEQ ID NO: 35 (Bac3Tag), SEQ ID NO: 22 (Bac4Tag) and SEQ ID NO: 12 (Bac5Tag); or variants thereof having at least 70% homology or identity thereto;
c) the binding partner of SEQ ID NO: 37 (QueenCatcher) and a peptide tag selected from SEQ ID NO: 47 (RumTrunkD9NTag), SEQ ID NO: 54 (Rum7Tag), SEQ ID NO: 56 (Rum3Tag), SEQ ID NO: 58 (Rum2Tag), SEQ ID NO: 60 (Rum4Tag), SEQ ID NO: 62 (Rum5Tag), SEQ ID NO: 64 (Rum6Tag), SEQ ID NO: 71 (RumTrunkTag), SEQ ID NO: 5 (SpyTag), SEQ ID NO: 7 (SdyTag), SEQ ID NO: 46 (RumTag), SEQ ID NO: 66 (BacTag); SEQ ID NO: 68 (Bac2Tag), SEQ ID NO: 35 (Bac3Tag), SEQ ID NO: 22 (Bac4Tag), SEQ ID NO: 12 (Bac5Tag) and SEQ ID NO: 76 (Clib9),
or variants thereof having at least 70% homology or identity thereto,
wherein a variant of a binding partner or of a peptide tag having at least 70% homology or identity to said binding partner or peptide tag is a functional variant which retains the capability of forming an isopeptide bond to the corresponding peptide tag or binding partner, respectively, and has at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity to said binding partner or peptide tag.

### Binding Efficacy

The peptide tags, modified binding partners and the binding peptide pairs described herein, or obtainable by the methods described herein, preferably have one or more improved properties compared to a reference.

One such property of interest is binding efficacy: as discussed above, the peptide tags and binding partners obtainable by the present methods preferably have increased binding efficacy. The peptide pairs described herein may also have increased binding efficacy, i.e. the at least one of the total binding and the binding rate of the peptide pair and binding partner of the peptide pair is increased compared to a reference peptide pair. The reference peptide pair preferably comprises either the same peptide tag or the same binding partner. The reference peptide pair thus is the pair comprising the binding partner (the first or second binding partner) which has most homology with the modified binding partner. Thus in preferred embodiments, the modified binding partner has more homology to the first binding partner than to the second binding partner, and the reference binding partner is the first binding partner; or the modified binding partner has more homology to the second binding partner than to the first binding partner and the reference binding partner is the second binding partner.

In some embodiments, the binding rate of the peptide pair is increased by at least 5%, such as at least 10%, such as at least 15%, such as at least 20%, such as at least 25%, such as at least 30%, such as at least 40%, such as at least 50%, such as at least 60%, such as at least 70%, such as at least 80%, such as at least 90%, such as at least 100%, or more compared to the reference peptide pair.

In some embodiments, the total binding of the peptide pair is increased by at least 5%, such as at least 10%, such as at least 15%, such as at least 20%, such as at least 25%, such as at least 30%, such as at least 40%, such as at least 50%, such as at least 60%, such as at least 70%, such as at least 80%, such as at least 90%, such as at least 100%, or more compared to the peptide pair.

In some embodiments, the binding rate of the peptide pair is increased by at least 5%, such as at least 10%, such as at least 15%, such as at least 20%, such as at least 25%, such as at least 30%, such as at least 40%, such as at least 50%, such as at least 60%, such as at least 70%, such as at least 80%, such as at least 90%, such as at least 100%, or more compared to the reference binding partner, and the total binding of the modified peptide partner is increased by at least 5%, such as at least 10%, such as at least 15%, such as at least 20%, such as at least 25%, such as at least 30%, such as at least 40%, such as at least 50%, such as at least 60%, such as at least 70%, such as at least 80%, such as at least 90%, such as at least 100%, or more compared to the reference peptide pair.

### Particle Formation

The peptide pairs described herein may also, alternatively or additionally, have increased ability to form a particle displaying a peptide of interest, such as a virus-like particle displaying a peptide of interest, wherein the particle comprises a particle-forming protein such as a virus-like particle-forming protein fused or conjugated to the binding partner and the compound of interest fused to the peptide tag, or wherein the particle comprises the virus-like particle-forming protein fused or conjugated to the peptide tag and the compound of interest fused to the binding partner, and wherein the particle is formed by spontaneous formation of the isopeptide bond between the binding partner and the peptide tag, when compared to the ability of the reference peptide pair to form a particle under similar conditions.

Such particles can form via particle-forming proteins, as is known in the art, for example, but not only, virus-like particle-forming proteins. By fusing one member of a peptide pair to such a protein and the other member of the peptide pair to a compound, for example a peptide, to be displayed on the particle, particles can be obtained which display the peptide. For example, a particle-forming protein is fused to a peptide tag, and the peptide to be displayed is fused to the corresponding binding partner. The spontaneous formation of an isopeptide bond between the peptide tag and its binding partner allows the peptide to be displayed on the surface of the particle. This technique has been used to generate virus-like particles (VLPs) displaying antigenic peptides, e.g. peptides associated with an abnormal physiological response such as a disease, as described in detail in WO 2016/112921. Capsid proteins are examples of suitable particle-forming proteins that can be used to generate such VLPs, as detailed herein above. For example, AP205, Q13, MS2, HBc, and phage fr, P22, Cowpea mosaic virus (CPMV), Brome mosaic virus (BMV), Cowpea chlorotic mottle virus (CCMV), Bacteriophage lambda Human adenovirus (AdV), Vault particle (PDB: 4V60). can be used. Other suitable proteins are known in the art, for example the proteins listed in Table 1 of Lieknina et al., 2019, can be used.

The method is however generally applicable to generate particles which are not virus-like particles. Other such examples of particle-forming proteins include: small heat-shock protein (HSP) (PDB: 1SHS), Apoferritin (PDB: 1DAT). Pyruvate dehydrogenase multienzyme complex (PDB: 1EAA), Thermosome (THS) and i301 (designed from the 2-keto-3-deoxy-phosphogluconate (KDPG) aldolase from the Entner—Doudoroff pathway of the hyperthermophilic bacterium Thermotoga maritima).

According to the present methods a protein, which can self-assemble into a nanoparticle, can be genetically modified by fusion of a peptide tag. The assembled nanoparticles (i.e. displaying the reactive peptide tag) can then be coupled to a peptide genetically fused to a binding partner capable of interacting with the peptide tag via an isopeptide bond when contacted with the peptide tag.

Any of the peptide tags, modified binding partners and peptide pairs described herein can advantageously be used to generate self-assembling nanoparticles.

In some embodiments, the peptide pair obtained by the present methods, when used to display a compound of interest such as an antigen, for example in a virus-like particle or in a particle as described below, can lead to an increased immune response when administered to a subject in need thereof, compared to the immune response obtained from a particle in which a reference peptide pair is used, for example compared to the immune response obtained using SpyCatcher/SpyTag (SEQ ID NO: 1 and SEQ ID NO: 5, respectively).

In some embodiments, the peptide pair consists of a modified binding partner as described herein, for example the binding partner of SEQ ID NO: 39 (MoonCake), the binding partner of SEQ ID NO: 41 (KatI) or the binding partner of SEQ ID NO: 37 (QueenCatcher), and of a suitable peptide tag, as described herein. In such embodiments, the reference peptide pair may consist of the same peptide tag and of a reference binding partner as described herein, for example SpyCatcher (SEQ ID NO: 1). In some embodiments, the reference peptide pair may consist of SpyCatcher/SpyTag (SEQ ID NO: 1 and SEQ ID NO: 5, respectively).

Herein is thus also disclosed a method for inducing an immune response in a subject in need thereof, comprising the administration of a particle such as a virus-like particle comprising the peptide pairs described herein or obtained by the methods described herein to the subject, where preferably the immune response is increased compared to the immune response obtained after administration of a particle such as a virus-like particle comprising a reference peptide pair, such as any of the reference peptide pairs described herein, but otherwise identical.

In some embodiments, the increased immune response is an increased IgM response and/or an increased IgG2 response, such as an increased IgG2a and/or IgG2b. In some embodiments, the increased immune response is an increased IgG1 response. In some embodiments, the increased immune response is an increased IgG3 response. Preferably, at least one of the IgM, IgG2a or IgG2b response is increased.

### Display of a Compound on a Particle

The peptide pairs described herein may also, alternatively or additionally, have an increased ability to display a compound of interest such as a peptide on a particle such as a virus-like particle, wherein the particle comprises a particle-forming protein such as a virus-like particle-forming protein fused or conjugated to the binding partner and the compound of interest fused or conjugated to the peptide tag, or wherein the particle comprises the particle-forming protein fused or conjugated to the peptide tag and the compound of interest fused or conjugated to the binding partner, and wherein the particle is formed by spontaneous formation of the isopeptide bond between the binding partner and the peptide tag, when compared to the ability of the reference peptide pair to display the peptide of interest under similar conditions.

Any of the peptide tags, modified binding partners and peptide pairs described herein can advantageously be used to generate self-assembling nanoparticles which can display a compound of interest.

Examples of particle-forming proteins include capsid proteins such as virus capsid proteins. For example, AP205, Q13, MS2, HBc, and phage fr, P22, Cowpea mosaic virus (CPMV), Brome mosaic virus (BMV), Cowpea chlorotic mottle virus (CCMV), Bacteriophage lambda Human adenovirus (AdV), Vault particle (PDB: 4V60). can be used. Other suitable proteins are known in the art, for example the proteins listed in Table 1 of Lieknina et al., 2019, can be used.

Other examples of particle-forming proteins, which are not virus capsid proteins, include: small heat-shock protein (HSP) (PDB: 1SHS), Apoferritin (PDB: 1DAT). Pyruvate dehydrogenase multienzyme complex (PDB: 1EAA), Thermosome (THS) and i301 (designed from the 2-keto-3-deoxy-phosphogluconate (KDPG) aldolase from the Entner—Doudoroff pathway of the hyperthermophilic bacterium Thermotoga maritima).

Examples of compounds of interest to be displayed on a surface (which can be an internal surface or an external surface) of the particle include peptides of interest, for example antigenic peptides. In some embodiments the antigenic peptide is capable of eliciting an immune reaction in an animal, such as a mammal, such as a cow, pig, horse, sheep, goat, llama, mouse, rat, monkey, most preferably such as a human being; or a bird such as a chicken, or fish such as a salmon.

In some embodiments, the compound of interest is an antigen associated with an abnormal physiological response, for example a disease or disorder such as disease is cancer, such as breast cancer, gastric cancer, ovarian cancer and uterine serous carcinoma; a cardiovascular disease, such as dyslipidemia, atherosclerosis, and/or hypercholesterolemia; an immune-inflammatory disease or a chronic disease, such as eosinophilic asthma, allergy, nasal polyposis, atopic dermatitis, eosinophilic esophagitis, hypereosinophilic syndrome, and Churg-Strauss syndrome, a neurological disease such as Alzheimer's disease; an infectious disease, such as an infectious disease selected from the group consisting of diseases caused by a virus, such as a coronavirus, for example SARS-CoV-2, malaria, tuberculosis, HIV and influenza; a lipid disorder such as hyperlipidemia, type I, type II, type III, type IV, or type V hyperlipidemia, secondary hypertriglyceridemia, hypercholesterolemia, familial hypercholesterolemia, xanthomatosis, cholesterol acetyltransferase deficiency; an arteriosclerotic condition such as atherosclerosis; or a coronary artery disease.

In some embodiments the antigen is a protein, peptide and/or an antigenic fragment from the group consisting of cancer-specific polypeptides, polypeptides associated with cardiovascular diseases, polypeptides associated with asthma, polypeptides associated with nasal polyposis, polypeptides associated with atopic dermatitis, polypeptides associated with eosinophilic esophagitis, polypeptides associated with hypereosinophilic syndrome, polypeptides associated with Churg-Strauss syndrome and/or polypeptides associated with pathogenic organisms.

The present compositions may thus be vaccine compositions with prophylactic applications. The present compositions may thus be useful for prophylaxis or treatment of a disease or disorder. The present compositions may also be useful for inducing an immune response in a subject by administering said compositions at least once to the subject.

### Polynucleotides

Also provided herein are polynucleotides encoding the modified binding partners obtainable by the methods disclosed herein. Also provided herein are polynucleotides encoding the peptide tags obtainable by the methods disclosed herein. The term polynucleotide herein refers to a nucleic acid molecule, preferably a DNA molecule, which encodes a given polypeptide, peptide or protein.

In some embodiments, the polynucleotide encodes a modified binding partner as described herein.

In some embodiments, the polynucleotide encodes the modified binding partner as set forth in SEQ ID NO: 37, said polynucleotide comprising or consisting of SEQ ID NO: 38 or a homologue thereof having at least 60% identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% identity thereto. Preferably, the codon encoding the residue corresponding to residue 31 of SEQ ID NO: 37 is not modified, i.e. the codon at position 91 to 93 encodes a lysine.

In some embodiments, the polynucleotide encodes the modified binding partner as set forth in SEQ ID NO: 39, said polynucleotide comprising or consisting of SEQ ID NO: 38 or a homologue thereof having at least 60% identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% identity thereto. Preferably, the codon encoding the residue corresponding to residue 31 of SEQ ID NO: 38 is not modified, i.e. the codon at position 91 to 93 encodes a lysine.

In some embodiments, the polynucleotide encodes the modified binding partner as set forth in SEQ ID NO: 41, said polynucleotide comprising or consisting of SEQ ID NO: 42 or a homologue thereof having at least 60% identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% identity thereto. Preferably, the codon encoding the residue corresponding to residue 31 of SEQ ID NO: 42 is not modified, i.e. the codon at position 91 to 93 encodes a lysine.

In some embodiments, the polynucleotide encodes the modified binding partner as set forth in SEQ ID NO: 29, or a homologue thereof having at least 60% identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% identity thereto. Preferably, the codon encoding the residue corresponding to residue 8 of SEQ ID NO: 29 is not modified, i.e. the codon at position 24 to 26 encodes a lysine.

In some embodiments, the polynucleotide encodes the modified binding partner as set forth in SEQ ID NO: 9, said polynucleotide comprising or consisting of SEQ ID NO: 10 or a homologue thereof having at least 60% identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% identity thereto. Preferably, the codon encoding the residue corresponding to residue 31 of SEQ ID NO: 10 is not modified, i.e. the codon at position 352 to 354 encodes an asparagine.

In some embodiments, the polynucleotide encodes the modified binding partner as set forth in SEQ ID NO: 31, said polynucleotide comprising or consisting of SEQ ID NO: 32 or a homologue thereof having at least 60% identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% identity thereto. Preferably, the codon encoding the residue corresponding to residue 31 of SEQ ID NO: 32 is not modified, i.e. the codon at position 91 to 93 encodes an aspartic acid.

In some embodiments, the polynucleotide encodes the peptide tag as set forth in SEQ ID NO: 46, said polynucleotide comprising or consisting of SEQ ID NO: 45 or a homologue thereof having at least 60% identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% identity thereto. Preferably, the codon encoding the residue corresponding to residue 12 of SEQ ID NO: 46 is not modified, i.e. the codon at position 37 to 39 encodes an aspartic acid.

In some embodiments, the polynucleotide encodes the peptide tag as set forth in SEQ ID NO: 48, said polynucleotide comprising or consisting of SEQ ID NO: 47 or a homologue thereof having at least 60% identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% identity thereto. Preferably, the codon encoding the residue corresponding to residue 9 of SEQ ID NO: 48 is not modified, i.e. the codon at position 25 to 27 encodes an asparagine.

In some embodiments, the polynucleotide encodes the peptide tag as set forth in SEQ ID NO: 50, said polynucleotide comprising or consisting of SEQ ID NO: 49 or a homologue thereof having at least 60% identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% identity thereto.

In some embodiments, the polynucleotide encodes the peptide tag as set forth in SEQ ID NO: 52, said polynucleotide comprising or consisting of SEQ ID NO: 51 or a homologue thereof having at least 60% identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% identity thereto.

In some embodiments, the polynucleotide encodes the peptide tag as set forth in SEQ ID NO: 54, said polynucleotide comprising or consisting of SEQ ID NO: 53 or a homologue thereof having at least 60% identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% identity thereto.

In some embodiments, the polynucleotide encodes the peptide tag as set forth in SEQ ID NO: 56, said polynucleotide comprising or consisting of SEQ ID NO: 55 or a homologue thereof having at least 60% identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% identity thereto.

In some embodiments, the polynucleotide encodes the peptide tag as set forth in SEQ ID NO: 58, said polynucleotide comprising or consisting of SEQ ID NO: 57 or a homologue thereof having at least 60% identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% identity thereto.

In some embodiments, the polynucleotide encodes the peptide tag as set forth in SEQ ID NO: 60, said polynucleotide comprising or consisting of SEQ ID NO: 59 or a homologue thereof having at least 60% identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% identity thereto.

In some embodiments, the polynucleotide encodes the peptide tag as set forth in SEQ ID NO: 62, said polynucleotide comprising or consisting of SEQ ID NO: 61 or a homologue thereof having at least 60% identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% identity thereto.

In some embodiments, the polynucleotide encodes the peptide tag as set forth in SEQ ID NO: 64, said polynucleotide comprising or consisting of SEQ ID NO: 63 or a homologue thereof having at least 60% identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% identity thereto.

In some embodiments, the polynucleotide encodes the peptide tag as set forth in SEQ ID NO: 66, said polynucleotide comprising or consisting of SEQ ID NO: 65 or a homologue thereof having at least 60% identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% identity thereto.

In some embodiments, the polynucleotide encodes the peptide tag as set forth in SEQ ID NO: 68, said polynucleotide comprising or consisting of SEQ ID NO: 67 or a homologue thereof having at least 60% identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% identity thereto. Preferably, the codon encoding the residue corresponding to residue 13 of SEQ ID NO: 68 is not modified, i.e. the codon at position 37 to 39 encodes an aspartic acid.

In some embodiments, the polynucleotide encodes the peptide tag as set forth in SEQ ID NO: 35, said polynucleotide comprising or consisting of SEQ ID NO: 36 or a homologue thereof having at least 60% identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% identity thereto.

In some embodiments, the polynucleotide encodes the peptide tag as set forth in SEQ ID NO: 22, said polynucleotide comprising or consisting of SEQ ID NO: 21 or a homologue thereof having at least 60% identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% identity thereto.

In some embodiments, the polynucleotide encodes the peptide tag as set forth in SEQ ID NO: 12, said polynucleotide comprising or consisting of SEQ ID NO: 11 or a homologue thereof having at least 60% identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% identity thereto.

In some embodiments, the polynucleotide encodes the peptide tag as set forth in SEQ ID NO: 71, said polynucleotide comprising or consisting of SEQ ID NO: 72 or a homologue thereof having at least 60% identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% identity thereto. Preferably, the codon encoding the residue corresponding to residue 9 of SEQ ID NO: 72 is not modified, i.e. the codon at position 25 to 27 of SEQ ID NO: 72 encodes an aspartic acid.

In some embodiments, the polynucleotide encodes the peptide tag as set forth in SEQ ID NO: 69, said polynucleotide comprising or consisting of SEQ ID NO: 70 or a homologue thereof having at least 60% identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% identity thereto.

In some embodiments, the polynucleotide encodes the peptide tag as set forth in SEQ ID NO: 75, said polynucleotide comprising or consisting of SEQ ID NO: 77 or a homologue thereof having at least 60% identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% identity thereto. Preferably, the codon encoding the residue corresponding to residue 8 of SEQ ID NO: 75 is not modified, i.e. the codon at position 22 to 24 of SEQ ID NO: 77 encodes an aspartic acid.

Said polynucleotides may be provided within a vector, i.e. a DNA molecule used as a vehicle to artificially carry foreign genetic material into a cell, where it can be replicated and/or expressed. The four major types of vectors are plasmids, viral vectors, cosmids, and artificial chromosomes. The vector itself is generally a DNA sequence that consists of an insert (transgene) and a larger sequence that serves as the “backbone” of the vector. The purpose of a vector which transfers genetic information to another cell is typically to isolate, multiply, or express the insert in the target cell. Expression vectors (expression constructs) specifically are for the expression of transgenes in target cells, and generally have a promoter sequence that drives expression of the transgene.

In the embodiments where the polynucleotide is a homologue of SEQ ID NO: 38, SEQ ID NO: 40, SEQ ID NO: 42, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 36, SEQ ID NO: 21, SEQ ID NO: 30, SEQ ID NO: 32, SEQ ID NO: 72 or SEQ ID NO: 11, the codon of the polynucleotide encoding the amino acid corresponding to one of the reactive residues involved in the formation of an isopeptide bond is preferably unchanged, or the sequence of the polynucleotide is such that the encoded polypeptide still comprises the reactive residue.

Vectors, such as plasmids, comprising the polynucleotides described herein are thus also provided. A vector may comprise several of the polynucleotides, as is known in the art. Herein are thus provided systems of polynucleotides, such as a system comprising two polynucleotides: a first polynucleotide encoding a binding partner as described herein, in particular the modified binding partners described herein, and a second polynucleotide encoding a peptide tag as described herein, in particular the peptide tags binding to a modified binding partner. In some embodiments, the system consists of two vectors: a first vector comprising a first polynucleotide encoding a binding partner as described herein, in particular the modified binding partners described herein, and a second vector comprising a second polynucleotide encoding a peptide tag as described herein, in particular the peptide tags binding to a modified binding partner.

In some embodiments, the present binding pairs (i.e. binding pairs consisting of a binding partner and a peptide tag, where the binding partner may be a known binding partner or a modified binding partner as described herein, and the peptide tag is a known peptide tag or a peptide tag as described herein) are used to couple two compounds, in particular two polypeptides. For example, the binding partner is fused to a first polypeptide and the peptide tag is fused to a second polypeptide—the skilled person knows how such fusions are obtained. Due to the formation of the isopeptide bond formation between the binding partner and the peptide tag, the two compounds (in our example the first and the second polypeptide) are brought into vicinity of one another. As detailed above, this can be taken advantage of to generate e.g. VLPs displaying an antigenic peptide, by fusing a capsid protein capable of forming a VLP to one of the binding partner and the peptide tag, and fusing the antigenic peptide to the other of the binding partner and the peptide tag.

The fusion can be achieved by designing a polynucleotide which encodes the first compound fused to the binding partner and another polynucleotide which encodes the second compound fused to the peptide tag. Alternatively, the fusion can be achieved by designing a polynucleotide encoding the first compound fused to the peptide tag and another polynucleotide encoding the second compound fused to the binding partner. A spacer or linker, in particular a glycine-serine based spacer or linker, may be introduced between the first compound and the binding partner or peptide tag, and/or between the second compound and the binding partner or peptide tag.

The compounds of interest may be fused to the peptide tag or binding partner via an N-terminal fusion or via a C-terminal fusion or via an internal fusion, e.g. in a loop.

### Host Cells

Also provided herein are host cells expressing the modified binding partners and/or the peptide tags disclosed herein. This can be done as is known in the art. The host cell comprises one or more of the polynucleotides described herein, encoding the modified binding partners or peptide tags described herein. The polynucleotides may be integrated into the genome of the host cells, or they may be provided as part of one or more vectors which is introduced in the cell by methods known in the art.

The polynucleotides may be codon-optimised to improve expression in the host cells, as is known in the art.

The host cell may be a bacterial cell, a yeast cell, a fungal cell, a plant cell, an animal cell, a mammalian cell or an insect cell.

In some embodiments the host cell may belong to one of the following: Escherichia coli, Spodoptera frugiperda (sf9), Trichoplusia ni (BTI-TN-5B1-4), Salmonella thyphimurium, Pichia Pastoris, Saccharomyces cerevisiae, Schizosaccharomyces pombe, Hansenula polymorpha, Drosophila Schneider 2 (S2), Lactococcus lactis, Xenopus laevis, Chinese hamster ovary (CHO), COS-1, HepG2, HeLA, BHK, Human Embryonic Kidney 293, Nicotiana tabacum cv. Samsun NN and Solanum tuberosum cv. Solara, a cell of the Nicotiana genus, a cell of the genus Solanum, a cell of the Lupinus genus, Lactuca sativa, a tomato cell such as Solanum lycopersicum, Glycine max, a CPMV cell, a PVX cell or a MagnICON cell, or Pseudomonas based systems. The cell may be a transgenic cell.

### Compositions

Also provided herein are compositions comprising:
i) A protein fused to a modified binding partner as disclosed herein, and a compound of interest such as a peptide, for example an antigen, fused to a peptide tag as disclosed herein; or
ii) A protein fused to a peptide tag as disclosed herein, and a compound of interest such as a peptide, for example an antigen, fused to a modified binding partner as disclosed herein,
wherein the modified binding partner and the peptide tag are capable of interacting by the spontaneous formation of an isopeptide bond, and
wherein the compound of interest and the protein are linked via an isopeptide bond between the modified binding partner and the peptide tag.

Some specific compositions comprise:
a) a particle-forming protein fused to a modified binding partner as described herein, and a compound of interest such as a peptide, for example an antigen fused to a peptide tag as described herein; or
b) a particle-forming protein fused to a peptide tag as described herein, and a compound of interest such as a peptide, for example an antigen fused to a modified binding partner as described herein;
wherein the modified binding partner and the peptide tag are capable of interacting by the spontaneous formation of an isopeptide bond, and
wherein the compound of interest and the particle-forming protein are linked via an isopeptide bond between the modified binding partner and the peptide tag, and
wherein the particle-forming protein and the compound of interest form a particle displaying said compound of interest.

Examples of particle-forming proteins include capsid proteins such as virus capsid proteins. For example, AP205, Q8, MS2, HBc, and phage fr, P22, Cowpea mosaic virus (CPMV), Brome mosaic virus (BMV), Cowpea chlorotic mottle virus (CCMV), Bacteriophage lambda Human adenovirus (AdV), Vault particle (PDB: 4V60). can be used. Other suitable proteins are known in the art, for example the proteins listed in Table 1 of Lieknina et al., 2019, can be used.

Other examples of particle-forming proteins, which are not virus capsid proteins, include: small heat-shock protein (HSP) (PDB: 1SHS), Apoferritin (PDB: 1DAT). Pyruvate dehydrogenase multienzyme complex (PDB: 1EAA), Thermosome (THS) and i301 (designed from the 2-keto-3-deoxy-phosphogluconate (KDPG) aldolase from the Entner—Doudoroff pathway of the hyperthermophilic bacterium Thermotoga maritima).

Examples of compounds of interest to be displayed on a surface (which can be an internal surface or an external surface) of the particle include peptides of interest, for example antigenic peptides. In some embodiments the antigenic peptide is capable of eliciting an immune reaction in an animal, such as a mammal, such as a cow, pig, horse, sheep, goat, llama, mouse, rat, monkey, most preferably such as a human being; or a bird such as a chicken, or fish such as a salmon.

In some embodiments, the compound of interest is an antigen associated with an abnormal physiological response, for example a disease or disorder such as disease is cancer, such as breast cancer, gastric cancer, ovarian cancer and uterine serous carcinoma; a cardiovascular disease, such as dyslipidemia, atherosclerosis, and/or hypercholesterolemia; an immune-inflammatory disease or a chronic disease, such as eosinophilic asthma, allergy, nasal polyposis, atopic dermatitis, eosinophilic esophagitis, hypereosinophilic syndrome, and Churg-Strauss syndrome, a neurological disease such as Alzheimer's disease; an infectious disease, such as an infectious disease selected from the group consisting of diseases caused by a virus, such as a coronavirus, for example SARS-CoV-2, malaria, tuberculosis, HIV and influenza; a lipid disorder such as hyperlipidemia, type I, type II, type III, type IV, or type V hyperlipidemia, secondary hypertriglyceridemia, hypercholesterolemia, familial hypercholesterolemia, xanthomatosis, cholesterol acetyltransferase deficiency; an arteriosclerotic condition such as atherosclerosis; or a coronary artery disease.

In some embodiments the antigen is a protein, peptide and/or an antigenic fragment from the group consisting of cancer-specific polypeptides, polypeptides associated with cardiovascular diseases, polypeptides associated with asthma, polypeptides associated with nasal polyposis, polypeptides associated with atopic dermatitis, polypeptides associated with eosinophilic esophagitis, polypeptides associated with hypereosinophilic syndrome, polypeptides associated with Churg-Strauss syndrome and/or polypeptides associated with pathogenic organisms.

The present compositions may thus be vaccine compositions with prophylactic applications. The present compositions may thus be useful for prophylaxis or treatment of a disease or disorder. The present compositions may also be useful for inducing an immune response in a subject by administering said compositions at least once to the subject.

Also provided herein is a method of manufacturing a pharmaceutical composition as described herein, comprising the steps of:
i) obtaining a first polypeptide comprising or consisting of a modified binding partner as described herein fused to a protein; and obtaining a second polypeptide comprising or consisting of a peptide tag as defined herein to a compound of interest; or
   obtaining a first polypeptide comprising or consisting of a peptide tag as described herein fused to a protein and obtaining a second polypeptide comprising or consisting of a modified binding partner as described herein fused to a compound of interest;
ii) contacting the first polypeptide and the second polypeptide, thereby allowing formation of an isopeptide bond between the peptide tag and the modified binding partner; and generating a pharmaceutical composition as described herein.

In particular embodiments, the method of manufacturing a composition comprises the steps of:
i) obtaining a first polypeptide comprising or consisting of a modified binding partner as described herein fused to a particle-forming protein; and
   obtaining a second polypeptide comprising or consisting of a peptide tag as described herein fused to a compound of interest such as a peptide; or obtaining a first polypeptide comprising or consisting of a peptide tag as described herein fused to a particle-forming protein and obtaining a second polypeptide comprising or consisting of a modified binding partner as described herein fused to a compound of interest;
ii) subjecting the first polypeptide to conditions which enable formation of particles;
iii) obtaining particles by formation of an isopeptide bond between the second polypeptide and the particle-forming protein of the first polypeptide; and
iv) generating a pharmaceutical composition comprising said particles, wherein the pharmaceutical composition is as described herein,
   thereby obtaining a pharmaceutical composition.

The particle-forming protein may be any of the particle-forming proteins listed herein above, for example a capsid protein.

### EXAMPLES

### Example 1

The modified binding partner set forth in SEQ ID NO: 37 was obtained starting from SEQ ID NO: 3. The reactive residue in SEQ ID NO: 3 is at position 31 of SEQ ID NO: 3, and is retained in the modified binding partner comprising or consisting of SEQ ID NO: 37 or the homologue thereof having at least 70% identity or homology thereto. The first reactive fragment from SEQ ID NO: 3 spans positions 1 to 93 of SEQ ID NO: 3. The modified binding partner of SEQ ID NO: 37 further comprises a fragment (the residual fragment) spanning positions 97 to 116 of SEQ ID NO: 1. The reactive residue 5 in SEQ ID NO: 37 is at position 31.

The modified binding partners set forth in SEQ ID NO: 39 and SEQ ID NO: 41 were both obtained starting from SEQ ID NO: 37. The modified binding partner set forth in SEQ ID NO: 39 and SEQ ID NO: 41 were designed by in silico structure modelling and rational design, introducing mutations in SEQ ID NO: 37. The reactive residue in SEQ ID NO: 39 and SEQ ID NO: 41 is at position 31 of these sequences.

### Example 2: Improved Characteristics of VLPs Obtained by Isopeptide Bond Formation Between Soluble Catcher and a Tag-VLP Fusion

In order to determine kinetics of coupling (EC50 coupling over time), soluble-catcher and VLP-tag (fusion of a VLP-forming protein and a tag) were diluted to 10 μM in PBS and mixed in a 1:1 ratio (5 uM final) and incubated at 37° C. for 1 min, 5 min, 10 min, 20 min, 40 min, 1 h, 1 h30 or 3 h and run on SDS gels. The reconstitution percentage was also determined; it is the endpoint binding after 3 hours, and was here determined as an average of two experiments.

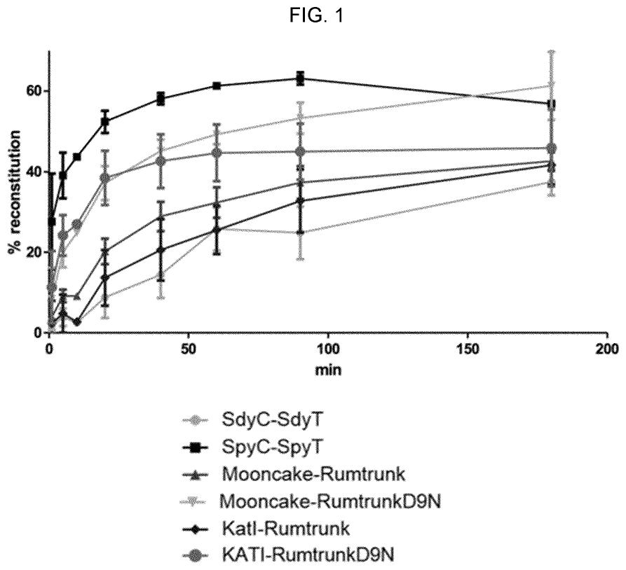

Results are shown in FIG. 1 and in table 1.

TABLE 1

| | EC50 on coupling | Percentage |
|---|---|---|
| Combination | over time | reconstitution |
| SdyCatcher + VLP- | 70.8 | 37.4 (+3.3) |
| SdyTag | | |
| Mooncake + VLP- | 24.6 | 61.3 (+8.5) |
| RumTrunkD9NTag | | |
| KATI + VLP- | 7.7 | 45.9 (+9.5) |
| RumTrunkD9NTag | | |
| SpyCatcher + VLP- | 8.4 | 68.6 |
| SpyTag | | |

As can be seen, kinetics for the Mooncake+VLP-RumTrunkD9NTag are improved compared to SdyCatcher+VLP-SdyTag, while they are essentially unchanged for the KATI+VLP-RumTrunkD9NTag compared to SpyCatcher+VLP-SpyTag. The percentage reconstitution is improved compared to SdyCatcher+VLP-SdyTag.

In order to determine maximal binding of the above VLPs, soluble-catcher and VLP-tag were respectively diluted to 20 μM and 10 μM in PBS and mixed in a 1:1 ratio (10 μM and 5 μM final) and incubated at 37° C. for 24 h and run on SDS gels. In order to determine the VLP yield, soluble-catchers were expressed in BL21 cells and grown in 2*YT media with ampicillin. A pre-culture was set overnight and transferred in 400 mL 2*YT media with ampicillin and expression of our protein was induced with 0.1 μM IPTG. After induction, overnight culture was spun down and the pellet was weighed (mg protein/gr pellet), protein was purified using IMAC and protein concentration was measured (mg/L). Results are shown in Table 2.

TABLE 2

| | Maximal binding | |
|---|---|---|
| | (percentage binding | VLP yield (mg/L) |
| Combination | after 24 h) | (mg protein/g pellet) |
| SdyCatcher + VLP- | 45.8 | 41.5 (2.81) |
| SdyTag | | |
| Mooncake + VLP- | 59 | 26.2 (1.55) |
| RumTrunkD9NTag | | |
| KATI + VLP- | 83.1 | 11.8 (0.7) |
| RumTrunkD9NTag | | |
| SpyCatcher + VLP- | ND | ND |
| SpyTag | | |

ND: not determined

Maximal binding is increased compared to SdyCatcher+VLP-SdyTag.

Taken together, these data show that the present VLPs have improved properties, in particular compared to SdyCatcher/SdyTag VLPs.

### Example 3: Improved Characteristics of Soluble Catcher and Soluble Tag Pairs

The experiments were performed as in example 1, unless indicated otherwise. Instead of a VLP-tag fusion, a soluble tag was used.

In order to determine kinetics, soluble-catcher and soluble-tag were diluted to 10 μM in PBS and mixed in a 1:1 ratio (5 μM final) and incubated at 37° C. for 1 min, 5 min, 10 min, 20 min, 40 min, 1 h, 1 h30 or 3 h and run on SDS gels. In order to determine the binding, soluble-catcher and soluble-tag were diluted to 10 μM in PBS and mixed in a 1:1 ratio (5 μM final) and incubated at 37° C. for 50 min and run on SDS gels. In order to determine maximal binding, soluble-catcher and soluble-tag were diluted to 20 μM and 10 μM, respectively, in PBS and mixed in a 1:1 ratio (10 μM and 5 μM final, respectively) and incubated at 37° C. for 24 h and run on SDS gels.

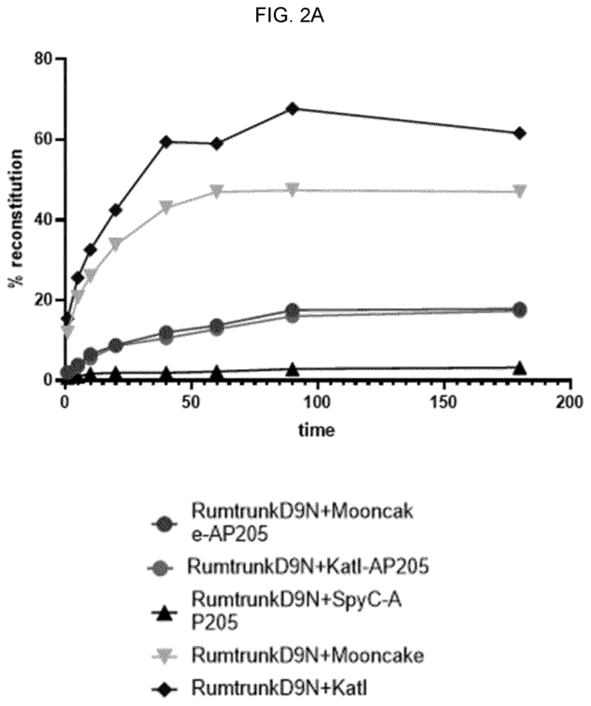

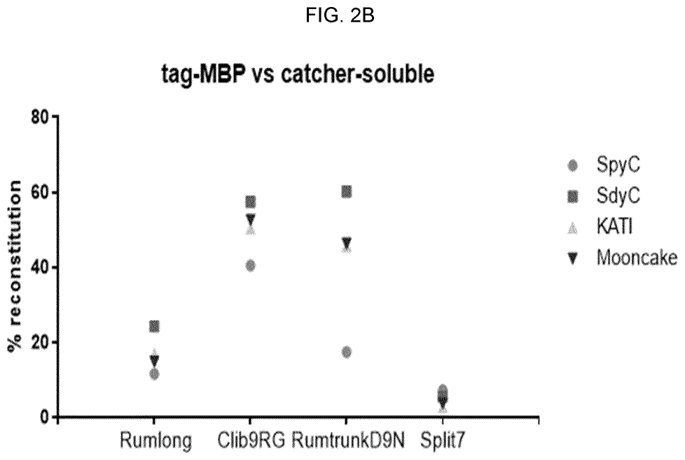

Kinetics, percentage binding (reconstitution after 50 minutes) and maximal binding for SdyC+SdyTag, Mooncake+MBP-RumTrunkD9NTag and KATI+MBP-RumTrunkD9NTag are shown in FIGS. 2A and 2B and table 3.

TABLE 3

| | EC50 on | Percentage | Maximal binding |
|---|---|---|---|
| | coupling | reconstitution | (percentage |
| Combination | over time | (50 min) | binding after 24 h) |
| SdyCatcher + | ND | ND | ND |
| SdyTag | | | |
| Mooncake + | 13.84 | 46.3 | 63.3 |
| MBP- | | | |
| RumTrunkD9NTag | | | |
| KATI + MBP- | 16.03 | 45.6 | 65.9 |
| RumTrunkD9NTag | | | |

ND: not determined

### Example 4: Improved VLP Display

In order to determine kinetics, VLP-catcher and soluble-tag were diluted to 10 μM in PBS and mixed in a 1:1 ratio (5 μM final) and incubated at 37° C. for 1 min, 5 min, 10 min, 20 min, 40 min, 1 h, 1 h30 or 3 h and run on SDS gels. In order to determine reconstitution (at 50 minutes, binding to RumTrunkD9NTag-MBP), soluble-catcher and VLP-tag were diluted to 10 μM in PBS and mixed in a 1:1 ratio (5 μM final) and incubated at 37° C. for 3 h and run on SDS gels. In order to determine maximal binding, VLP-catcher and soluble-tag were respectively diluted to 20 μM and 10 μM in PBS and mixed in a 1:1 ratio (10 μM and 5 μM final, respectively) and incubated at 37° C. for 24 h and run on SDS gels. To determine the VLP yield, the VLP-catcher fusions were expressed in BL21 cells and grown in 2*YT media with ampicillin. A pre-culture was set overnight and transferred in 400 mL 2*YT media with ampicillin and expression of our protein was induced with 0.1 μM IPTG. After induction, overnight culture was spun down and pellet was weighed (mg protein/gr pellet), protein was purified using an optiprep gradient and spinned down in ultracentrifuge. VLP concentration was measured by BCA assay (mg/L).

Results are shown in Tables 4 and 5.

TABLE 4

| | EC50 on coupling | Percentage |
|---|---|---|
| Combination | over time | reconstitution |
| SdyCatcher-VLP + | ND | ND |
| RumTrunkD9NTag | | |
| Mooncake-VLP + | 33.01 | 21.3 |
| RumTrunkD9NTag | | |
| KATI-VLP + | 41.95 | 16.6 |
| RumTrunkD9NTag | | |
| SpyCatcher-VLP + | ND | 11.9 |
| RumTrunkD9NTag | | |

TABLE 5

| | Maximal binding | |
|---|---|---|
| | (percentage binding | VLP yield (mg/L) |
| Combination | after 24 h) | (mg protein/g pellet) |
| SdyCatcher-VLP + | ND | Yield too low |
| RumTrunkD9NTag | | |
| Mooncake-VLP + | 35.5 | 164.8 (11.6) |
| RumTrunkD9NTag | | |
| KATI-VLP + | 36.1 | 103.1 (7.3) |
| RumTrunkD9NTag | | |
| SpyCatcher-VLP + | ND | 108.6 (7.6) |
| RumTrunkD9NTag | | |

ND: not determined

### Example 5: Increased Immune Response

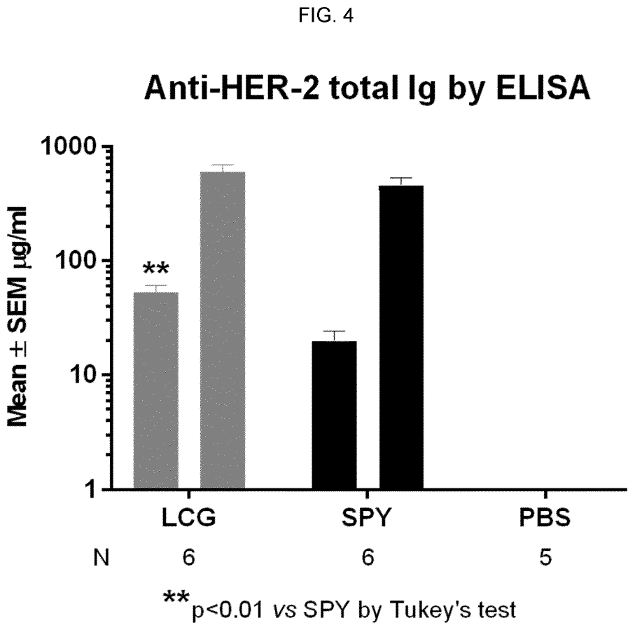

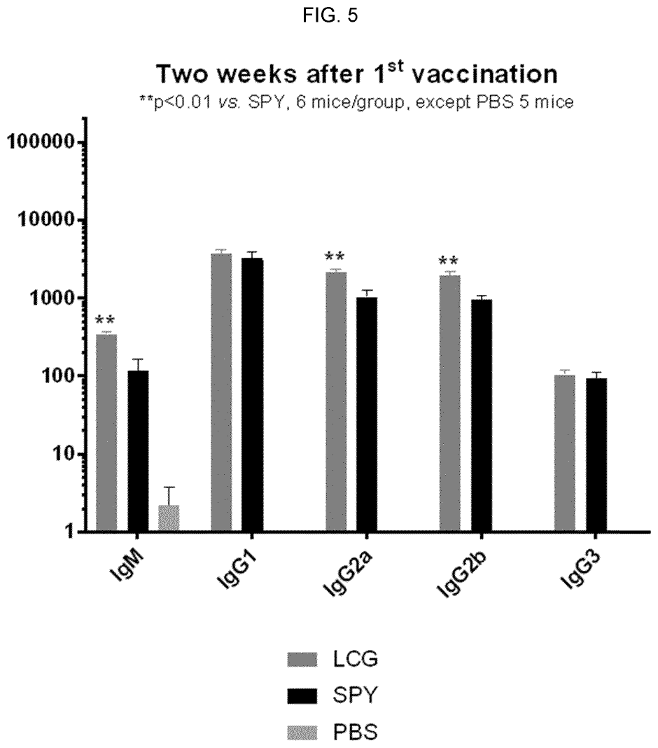

Groups of mice (n=6) were immunized prime-boost with an equal dose (6 mcg) of MoonCake-HER2 VLP (LCG), SpyCatcher-HER2 VLP (SPY), or PBS respectively (for the PBS group, n=5). Serum was obtained two weeks after each immunization and levels of antigen-specific IgM and IgG (subclasses 1, 2a, 2b and 3) were measured by ELISA. Immunization with MoonCake-HER2 VLP induced significantly higher antigen-specific total Ig compared to SpyCatcher-HER2 VLP (FIG. 4). Immunization with MoonCake-HER2 VLP induced significantly higher IgM as well as IgG2a and IgG2b-compared to SpyCatcher-HER2 VLP (FIG. 5). The two vaccines had comparable coupling efficiency (i.e. number of HER2 displayed per VLP) and monodispersity, as measured by SDS-PAGE densitometry and dynamic light-scattering analysis, respectively.

Sequences

| | | | Position of reactive |
|---|---|---|---|
| | Organism of | | residue involved (or |
| | origin or | | suspected to be |
| SEQ | artificial | | involved) in isopep- |
| ID NO: | sequence | Description | tide bond formation |
| 1 | Streptococcus | SpyCatcher | 34 |
| | pyogenes | | |
| 2 | Streptococcus | SpyCatcher DNA | — |
| | pyogenes | | |
| 3 | Streptococcus | SdyCatcher | 31 |
| | dysgalactiae | | |
| 4 | Streptococcus | SdyCatcher DNA | — |
| | dysgalactiae | | |
| 5 | Streptococcus | SpyTag | |
| | pyogenes | | |
| 6 | Streptococcus | SpyTag DNA | |
| | pyogenes | | |
| 7 | Streptococcus | SdyTag | 7 |
| | dysgalactiae | | |
| 8 | Streptococcus | SdyTag DNA | |
| | dysgalactiae | | |
| 9 | Streptococcus | SnoopCatcher | 117 |
| | pneumoniae | | |
| 10 | Streptococcus | SnoopCatcher | |
| | pneumoniae | DNA | |
| 11 | Bacillus cereus | Bac5Tag DNA | |
| | VD022 | | |
| 12 | Bacillus cereus | Bac5Tag | |
| | VD022 | (hypothetical | |
| | | protein IC1_02949, | |
| | | partial, | |
| | | EJP89589.1) | |
| 13 | Actinomyces | FimP | 13 |
| | viscosus | domain 3 | |
| 14 | Actinomyces | FimP domain 3 | |
| | viscosus | DNA | |
| 15 | Streptococcus | Streptococcal | 10 |
| | pneumonia | ancillary pilin | |
| | serotype 4 | Domain 2 | |
| | (strain ATCC | | |
| | BAA-334/TIGR4) | | |
| 16 | Streptococcus | Streptococcal | |
| | pneumonia | ancillary pilin | |
| | serotype 4 | Domain 2 DNA | |
| | (strain ATCC | | |
| | BAA-334/TIGR4) | | |
| 17 | Streptococcus | Streptococcal | 9 |
| | pneumonia | ancillary pilin | |
| | serotype 4 | Domain 3 | |
| | (strain ATCC | | |
| | BAA-334/TIGR4) | | |
| 18 | Streptococcus | Streptococcal | |
| | pneumonia | ancillary pilin | |
| | serotype 4 | Domain 3 DNA | |
| | (strain ATCC | | |
| | BAA-334/TIGR4) | | |
| 19 | Corynebacterium | Major Pilin SpaD | 12 |
| | diphtheriae | Domain 3 | |
| | NCTC 13129 | | |
| 20 | Corynebacterium | Major Pilin SpaD | |
| | diphtheriae | Domain 3 DNA | |
| | NCTC 13129 | | |
| 21 | Bacillus cereus | Bac4Tag DNA | |
| 22 | Bacillus cereus | Bac4Tag (choice- | |
| | | of-anchor A family | |
| | | protein) | |
| | | (WP_088344882.1) | |
| 23 | Lactobacillus | Pilin subunit | 14 |
| | rhamnosus GG | (SpaA) domain 2 | |
| 24 | Lactobacillus | Pilin subunit | |
| | rhamnosus GG | (SpaA) domain 2 | |
| | | DNA | |
| 25 | Lactobacillus | Pilin subunit | 9 |
| | rhamnosus GG | (SpaA) domain 3 | |
| 26 | Lactobacillus | Pilin subunit | |
| | rhamnosus GG | (SpaA) domain 3 | |
| | | DNA | |
| 27 | Streptococcus | Surface protein | 7 |
| | agalactiae A909 | Spb1 domain 3 | |
| 28 | Streptococcus | Surface protein | |
| | agalactiae A909 | Spb1 domain 3 | |
| | | DNA | |
| 29 | Streptococcus | PsCsCatcher | 8 |
| | intermedius | | |
| 30 | Streptococcus | PsCsCatcher | |
| | intermedius | (LPXTG cell wall | |
| | | anchor domain- | |
| | | containing protein) | |
| | | DNA | |
| 31 | Streptococcus | RgA Catcher | 9 |
| | pneumoniae | | |
| 32 | Streptococcus | RgA Catcher (cell | |
| | pneumoniae | wall surface anchor | |
| | | family protein) DNA | |
| 33 | Corynebacterium | Major Pilin SpaD | 12 |
| | diphtheriae | Domain 1 | |
| | NCTC 13129 | | |
| 34 | Corynebacterium | Major Pilin SpaD | |
| | diphtheriae | Domain 1 DNA | |
| | NCTC 13129 | | |
| 35 | Bacillus cereus | Bac3Tag (choice- | |
| | | of-anchor A family | |
| | | protein) (NCBI | |
| | | WP_053565148.1) | |
| 36 | Bacillus cereus | Bac3Tag DNA | |
| 37 | Artificial | QueenCatcher | 31 |
| | sequence | | |
| 38 | Artificial | QueenCatcher | |
| | sequence | DNA | |
| 39 | Artificial | MoonCake | 31 |
| | sequence | | |
| 40 | Artificial | MoonCake DNA | |
| | sequence | | |
| 41 | Artificial | Kat I | 31 |
| | sequence | | |
| 42 | Artificial | Kat I DNA | |
| | sequence | | |
| 43 | Artificial | Reference motif | GxxxFVMxDx |
| | sequence | | |
| 44 | Artificial | Reference motif | GxxxVVMxDx |
| | sequence | | |
| 45 | Ruminococcus | RumTag DNA | |
| | sp. AF26-25AA | | |
| 46 | Ruminococcus | RumTag (Cna B- | 12 |
| | sp. AF26-25AA | type domain- | |
| | | containing protein) | |
| 47 | Artificial | Rum TrunkD9NTag | 9 |
| | sequence | | |
| 48 | Artificial | RumtrunkD9NTag | |
| | sequence | DNA | |
| 49 | Streptococcus | PhoTag DNA | |
| | phocae | | |
| 50 | Streptococcus | PhoTag (LPXTG | |
| | phocae | cell wall anchor | |
| | | domain-containing | |
| | | protein) (NCBI | |
| | | Reference | |
| | | Sequence: | |
| | | WP_082385550.1) | |
| 51 | Enterococcus | EntTag DNA | |
| | faecalis | | |
| 52 | Enterococcus | EntTag | |
| | faecalis | (hypothetical | |
| | | protein | |
| | | CUN42_14770, | |
| | | partial, GenBank | |
| | | PQC83400.1) | |
| 53 | Ruminococcus | Rum7Tag DNA | |
| | sp. AM43-6 | | |
| 54 | Ruminococcus | Rum7Tag (Cna B | |
| | sp. AM43-6 | domain protein) | |
| | | (NCBI | |
| | | WP_118125159.1) | |
| 55 | Ruminococcus | Rum3Tag DNA | |
| | sp. Marseille- | | |
| | P6503 | | |
| 56 | Ruminococcus | Rum3Tag (TonB- | |
| | sp. Marseille- | dependent | |
| | P6503 | receptor) | |
| 57 | Ruminococcus | Rum2Tag DNA | |
| | sp. AF25-19 | | |
| 58 | Ruminococcus | Rum2Tag | |
| | sp. AF25-19 | (hypothetical | |
| | | protein | |
| | | DWY44_14000, | |
| | | partial) | |
| 59 | Ruminococcus | Rum4Tag DNA | |
| | sp. | | |
| 60 | Ruminococcus | Rum4Tag | |
| | sp. | (hypothetical | |
| | | protein) (NCBI | |
| | | HCW12338.1) | |
| 61 | Ruminococcus | Rum5Tag DNA | |
| | flavefaciens | | |
| 62 | Ruminococcus | Rum5Tag (Cna B- | |
| | flavefaciens | type domain- | |
| | | containing protein) | |
| | | (NCBI | |
| | | WP_051536060.1) | |
| 63 | Ruminococcus | Rum6Tag DNA | |
| | sp. | | |
| 64 | Ruminococcus | Rum6Tag | |
| | sp. | | |
| 65 | Bacillus cereus | BacTag DNA | |
| 66 | Bacillus cereus | BacTag (choice-of- | |
| | | anchor A family | |
| | | protein) (NCBI | |
| | | WP_080470427.1) | |
| 67 | Bacillus cereus | Bac2Tag | |
| 68 | Bacillus cereus | Bac2Tag | 13 |
| | | (hypothetical | |
| | | protein | |
| | | COD21_31890, | |
| | | partial) (NCBI | |
| | | PGT97799.1) | |
| 69 | Streptococcus | SnoopTag | |
| | pneumoniae | | |
| 70 | Streptococcus | SnoopTag DNA | |
| | pneumoniae | | |
| 71 | Ruminococcus | RumTrunkTag | 9 |
| | sp. | | |
| 72 | Ruminococcus | RumTrunkTag | |
| | sp. | DNA | |
| 73 | Artificial | Reference motif | GxxxIVMxDx |
| | sequence | | |
| 74 | Artificial | Reference motif | GxxxYVMxDx |
| | sequence | | |
| 75 | Streptococcus | PsCsTag protein | 8 |
| | intermedius | | |
| 76 | Artificial | Clib9 | |
| | sequence | | |
| 77 | Streptococcus | PsCsTag DNA | |
| | intermedius | | |
| 78 | Artificial | Clib9 DNA | |
| | sequence | | |
| 79 | Artificial | MoonCake | 35 |
| | sequence | | |
| 80 | Artificial | Katl | 35 |
| | sequence | | |

### REFERENCES

Lieknina I, Kalninš G, Akopjana I, Bogans J, Šišovs M, Jansons J, Rümnieks J, Tärs K (2019) Production and characterization of novel ssRNA bacteriophage virus-like particles from metagenomic sequencing data. J Nanobiotechnology 17(1):61. doi: 10.1186/s12951-019-0497-8.
Kang et al. Stabilizing isopeptide bonds revealed in gram-positive bacterial pilus structure Science. 2007 Dec. 7; 318(5856):1625-8.
Schwarz-Linek et al. Yet more intramolecular cross-links in Gram-positive surface proteins. PNAS Jan. 28, 2014 111 (4) 1229-1230;
https://doi.org/10.1073/pnas.1322482111

### Items

1. A method of producing a modified binding partner capable of binding to a peptide tag via the spontaneous formation of an isopeptide bond between one reactive residue comprised within said modified binding partner and another reactive residue comprised within said peptide tag, said method comprising the steps of:
   i) Selecting at least a first pair of peptides consisting of a first peptide tag and a first binding partner and a second pair of peptides consisting of a second peptide tag and a second binding partner, wherein for each pair of peptides the peptide tag and the binding partner are capable of or suspected of being capable of binding to each other by spontaneously forming an isopeptide bond;
   ii) Identifying the position of the isopeptide bond for the first pair of peptides and/or the second pair of peptides, thereby identifying a first reactive fragment of the first binding partner and/or a second reactive fragment of the second binding partner, and a first residual fragment of the first binding partner and/or a second residual fragment of the second binding partner; wherein the first and/or second reactive fragment comprises the reactive residue involved in the isopeptide bond;
   iii) Designing the modified binding partner, wherein the modified binding partner comprises or consists of i) the first reactive fragment, or a homologue thereof having at least 70% homology thereto, and the second residual fragment, or a homologue thereof having at least 70% homology thereto, wherein the first reactive fragment preferably is upstream of the second residual fragment, wherein the modified binding partner does not comprise both reactive residues involved in the formation of the isopeptide bond;
   iv) Producing the modified binding partner.
2. The method according to item 1, further comprising the steps of:
   v) Determining one or more binding properties of the modified binding partner, wherein said one or more properties are preferably selected from i) the total binding and ii) the binding rate of the modified binding partner to one or more of the first peptide tag, the second peptide tag or a third peptide tag;
   vi) Determining the corresponding one or more binding properties of the first binding partner and/or of the second binding partner to one or more of the first peptide tag, the second peptide tag or the third peptide tag;
   vii) Comparing the binding properties determined in steps e) and f), wherein an increased binding efficacy of the modified partner to any one or more of the first peptide tag, the second peptide tag or the third peptide tag compared to the binding efficacy of the first and/or second binding partner to the same tag is indicative of the modified binding partner having improved properties compared to the first and/or second binding partners, wherein the binding efficacy is increased if at least one of the total binding and the binding rate is increased compared to the binding efficacy of the first and/or second binding partners, preferably compared to the binding efficacy of the first binding partner.
3. The method according to any one of the preceding items, wherein an increase in the binding rate of the modified binding partner to at least one of said first, second and third peptide tag, compared to the binding rate of at least one of the first and/or second binding partner to the same peptide tag, is indicative of the modified binding partner having increased binding efficacy compared to the first and/or second binding partner, preferably compared to the binding efficacy of the first binding partner, preferably wherein said increase is at least 5%, such as at least 10%, such as at least 15%, such as at least 20%, such as at least 25%, such as at least 30%, such as at least 40%, such as at least 50%, such as at least 60%, such as at least 70%, such as at least 80%, such as at least 90%, such as at least 100%, or more.
4. The method according to any one of the preceding items, wherein an increase in total binding of the modified binding partner to at least one of said first, second and third peptide tag, compared to the total binding of at least one of the first and/or second binding partner to the same peptide tag, is indicative of the modified binding partner having increased binding efficacy compared to the first and/or second binding partner, preferably compared to the binding efficacy of the first binding partner, preferably wherein said increase is at least 5%, such as at least 10%, such as at least 15%, such as at least 20%, such as at least 25%, such as at least 30%, such as at least 40%, such as at least 50%, such as at least 60%, such as at least 70%, such as at least 80%, such as at least 90%, such as at least 100%, or more.
5. The method according to any one of the preceding items, wherein the modified binding partner has a length of 5 amino acids or more, such as 10 amino acids or more, such as 15 amino acids or more, such as 20 amino acids or more, such as 25 amino acids, such as 30 amino acids, such as 35 amino acids, such as 40 amino acids, such as 45 amino acids, such as 50 amino acids, such as 60, 70, 80, 90, 100, 125, 150, 175, 200, 225, 250, 275, 300, 325 or 350 amino acids or more.
6. The method according to any one of the preceding items, wherein the first and/or the second binding partner are independently selected from SEQ ID NO: 1 (SpyCatcher), SEQ ID NO: 3 (SdyCatcher), SEQ ID NO: 9 (SnoopCatcher), SEQ ID NO: 13, SEQ ID NO: 15, SEQ ID NO: 17, SEQ ID NO: 19, SEQ ID NO: 23, SEQ ID NO: 25, SEQ ID NO: 27, SEQ ID NO: 29, SEQ ID NO: 30, SEQ ID NO: 31, SEQ ID NO: 71 and SEQ ID NO: 33 and homologues thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto.
7. The method according to any one of the preceding items, wherein the first peptide tag, the second peptide tag and/or the third peptide tag are independently selected from the group consisting of SEQ ID NO: 46, SEQ ID NO: 5 (SpyTag), SEQ ID NO: 7 (SdyTag), SEQ ID NO: 69 (SnoopTag), SEQ ID NO: 46 (RumTag), SEQ ID NO: 47 (RumTrunkD9NTag), SEQ ID NO: 50 (PhoTag), SEQ ID NO: 52 (EntTag), SEQ ID NO: 54 (Rum7Tag), SEQ ID NO: 56 (Rum3Tag), SEQ ID NO: 58 (Rum2Tag), SEQ ID NO: 60 (Rum4Tag), SEQ ID NO: 62 (Rum5Tag), SEQ ID NO: 64 (Rum6Tag), SEQ ID NO: 66 (BacTag), SEQ ID NO: 68 (Bac2Tag), SEQ ID NO: 35 (Bac3Tag), SEQ ID NO: 22 (Bac4Tag), SEQ ID NO: 31 and SEQ ID NO: 12 (Bac5Tag), or homologues thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto.
8. The method according to any one of the preceding items, wherein the reactive residue in the first, second or third peptide tag is an aspartate or an asparagine residue, and/or wherein the reactive residue in the first, second and/or the modified binding partner is a lysine or an asparagine residue.
9. A modified binding partner obtainable by the method according to any one of the preceding items.
10. A modified binding partner capable of binding to a peptide tag via the spontaneous formation of an isopeptide bond between one reactive residue comprised within said modified binding partner and another reactive residue comprised within said peptide tag, wherein the modified binding partner does not comprise both reactive residues involved in the formation of the isopeptide bond, and wherein the modified binding partner comprises or consists of a first reactive fragment of a first binding partner comprising one reactive residue capable of interacting with a first peptide tag comprising another reactive residue via the formation of an isopeptide bond between the reactive residues, or a homologue thereof having at least 70% homology thereto, and a second residual fragment of a second binding partner, wherein said second binding partner is capable of interacting with a second peptide tag comprising another reactive residue via the formation of an isopeptide bond between the reactive residues, wherein the second residual fragment does not comprise the reactive residue, or a homologue thereof having at least 70% homology thereto, preferably wherein the first reactive fragment is upstream of the second residual fragment.
11. The modified binding partner according to any one of items 9 to 10, wherein the modified binding partner is obtained by the method according to any one of items 1 to 8.
12. The modified binding partner according to any one of items 9 to 11, wherein the first and the second binding partner are independently selected from SEQ ID NO: 1 (SpyCatcher), SEQ ID NO: 3 (SdyCatcher), SEQ ID NO: 9 (SnoopCatcher), SEQ ID NO: 13, SEQ ID NO: 15, SEQ ID NO: 17, SEQ ID NO: 19, SEQ ID NO: 23, SEQ ID NO: 25, SEQ ID NO: 27, SEQ ID NO: 29, SEQ ID NO: 30, SEQ ID NO: 31 and SEQ ID NO: 33 and homologues thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto.
13. The modified binding partner according to any one of items 9 to 12, comprising or consisting of SEQ ID NO: 37, SEQ ID NO: 39, SEQ ID NO: 29, SEQ ID NO: 71 or SEQ ID NO: 41, or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto.
14. The modified binding partner according to any one of items 9 to 13, having an increased binding efficacy to a reference peptide tag, compared to the binding efficacy to any one or more of the binding efficacy of a reference binding partner to the same reference peptide tag, wherein the reference peptide tag is a first peptide tag, a second peptide tag or a third peptide tag as defined in any one of items 1 to 8, and the reference binding partner is the first binding partner or the second binding partner as defined in any one of items 1 to 8, and wherein the binding efficacy is increased if at least one of the total binding and the binding rate is increased compared to the binding efficacy of the reference binding partner, preferably compared to the binding efficacy of the first binding partner.
15. The modified binding partner according to any one of items 9 to 14, wherein the binding rate of the modified peptide partner is increased by at least 5%, such as at least 10%, such as at least 15%, such as at least 20%, such as at least 25%, such as at least 30%, such as at least 40%, such as at least 50%, such as at least 60%, such as at least 70%, such as at least 80%, such as at least 90%, such as at least 100%, or more compared to the reference binding partner.
16. The modified binding partner according to any one of items 9 to 15, wherein the total binding of the modified peptide partner is increased by at least 5%, such as at least 10%, such as at least 15%, such as at least 20%, such as at least 25%, such as at least 30%, such as at least 40%, such as at least 50%, such as at least 60%, such as at least 70%, such as at least 80%, such as at least 90%, such as at least 100%, or more compared to the reference binding partner.
17. The modified binding partner according to any one of items 9 to 15, wherein the first peptide tag, the second peptide tag and/or the third peptide tag are independently selected from the group consisting of SEQ ID NO: 5 (SpyTag), SEQ ID NO: 7 (SdyTag), SEQ ID NO: 69 (SnoopTag), SEQ ID NO: 46 (RumTag), SEQ ID NO: 47 (RumTrunkD9NTag), SEQ ID NO: 50 (PhoTag), SEQ ID NO: 52 (EntTag), SEQ ID NO: 54 (Rum7Tag), SEQ ID NO: 56 (Rum3Tag), SEQ ID NO: 58 (Rum2Tag), SEQ ID NO: 60 (Rum4Tag), SEQ ID NO: 62 (Rum5Tag), SEQ ID NO: 64 (Rum6Tag), SEQ ID NO: 66 (BacTag), SEQ ID NO: 68 (Bac2Tag), SEQ ID NO: 35 (Bac3Tag), SEQ ID NO: 22 (Bac4Tag), SEQ ID NO: 46, SEQ ID NO: 31 and SEQ ID NO: 12 (Bac5Tag), or homologues thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto, preferably the first peptide tag, the second peptide tag and/or the third peptide tag are independently selected from the group consisting of SEQ ID NO: 46 (RumTag), SEQ ID NO: 5 (SpyTag) and SEQ ID NO: 7 (SdyTag).
18. A method of producing a peptide tag capable of binding to a binding partner via the spontaneous formation of an isopeptide bond between one reactive residue comprised within said peptide tag and another reactive residue comprised within said binding partner, preferably wherein said binding partner is a modified binding partner according to any one of items 9 to 14, said method comprising the steps of:
   a) Identifying candidate peptide tags having at least 60% similarity to a reference peptide tag, wherein the reference peptide tag is capable of spontaneously forming an isopeptide bond with at least one reference binding partner, preferably wherein the reference peptide tag comprises a reference binding motif;
   b) Selecting peptide tags from the candidate peptide tags identified in a), wherein the selected peptide tags comprise at least one reactive residue potentially involved in the formation of the isopeptide bond;
   c) Designing and producing the peptide tag from the selected peptide tags, wherein each peptide tag comprises or consists of a fragment of the selected peptide tags spanning from 4 to 24 amino acids upstream to 2 to 22 amino acids downstream of the reactive residue potentially involved in the formation of the isopeptide bond, or a homologue thereof having at least 70% homology thereto, with the proviso that the homologue comprises the reactive residue.
19. The method according to item 18, further comprising step d) of mutating the reactive residue, preferably wherein the reactive residue is an aspartate, which is mutated to an asparagine, wherein step d) is performed after step c).
20. The method according to any one of items 18 to 19, wherein the candidate peptide tags comprise a sequence motif within 50 amino acids from their C-terminus, such as within 45, 40, 35, 30 or 25 amino acids from their C-terminus.
21. The method according to item 20, wherein the sequence motif comprises or consists of SEQ ID NO: 73 (GX₁X₂X₃IVMX₄DX₅), SEQ ID NO: 74 (GX₁X₂X₃YVMX₄DX₅), SEQ ID NO: 43 (GX₁X₂X₃FVMX₄DX₅) or SEQ ID NO: 73 (GX₁X₂X₃WVMX₄DX₅).
22. A peptide tag comprising or consisting of a fragment of a protein comprising at least one reactive residue involved in the formation of an isopeptide bond between said peptide tag and a binding partner, wherein the peptide tag comprises or consists of a fragment of said protein spanning from 4 to 24 amino acids upstream to 2 to 22 amino acids downstream of the reactive residue, or a homologue thereof having at least 70% homology thereto, with the proviso that the homologue comprises the reactive residue, preferably wherein the reactive residue is an asparagine or an aspartate.
23. The peptide tag according to item 22, comprising or consisting of SEQ ID NO: 46 (RumTag), SEQ ID NO: 47 (RumTrunkD9NTag), SEQ ID NO: 50 (PhoTag), SEQ ID NO: 52 (EntTag), SEQ ID NO: 54 (Rum7Tag), SEQ ID NO: 56 (Rum3Tag), SEQ ID NO: 58 (Rum2Tag), SEQ ID NO: 60 (Rum4Tag), SEQ ID NO: 62 (Rum5Tag), SEQ ID NO: 64 (Rum6Tag), SEQ ID NO: 66 (BacTag), SEQ ID NO: 68 (Bac2Tag), SEQ ID NO: 35 (Bac3Tag), SEQ ID NO: 22 (Bac4Tag), SEQ ID NO: 29, SEQ ID NO: 31 and SEQ ID NO: 12 (Bac5Tag) and homologues thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto.
24. The peptide tag according to any one of items 22 to 23, wherein the peptide tag comprises a sequence motif comprising or consisting of SEQ ID NO: 73, preferably within 20 amino acids of its C-terminus, such as within 19, 18, 17, 16, 15, 14, 13, 12, 11, 10, 9, 8, 7, 6 or 5 amino acids of its C-terminus.
25. A method of producing a peptide pair comprising or consisting of a modified binding partner and a peptide tag, wherein the modified binding partner is capable of binding to the peptide tag via the spontaneous formation of an isopeptide bond between one reactive residue comprised within said modified binding partner and another reactive residue comprised within said peptide tag, said method comprising the steps of:
   i) producing a modified binding partner by the method according to any one of items 1 to 8; and/or
   ii) producing a peptide tag by the method according to any one of items 18 to 21.
26. A peptide pair comprising or consisting of a peptide tag as defined in any one of the preceding items and a modified binding partner as defined in any one of the preceding items.
27. The peptide pair according to item 26, wherein the peptide pair is selected from:
   a) SEQ ID NO: 9 and SEQ ID NO: 69
   b) SEQ ID NO: 47 and SEQ ID NO: 39; and
   c) SEQ ID NO: 47 and SEQ ID NO: 41;
   d) SEQ ID NO: 46 and SEQ ID NO: 39;
   e) SEQ ID NO: 46 and SEQ ID NO: 41.
28. A modified binding partner having one or more improved properties compared to a reference binding partner, wherein the one or more improved properties are independently selected from one or more of:
a) increased binding efficacy of the modified binding partner to a peptide tag relative to the binding of the reference binding partner to said peptide tag, wherein said modified binding partner and optionally said reference binding partner are capable of binding to said peptide tag via the spontaneous formation of an isopeptide bond between one reactive residue comprised within said modified binding partner or within said reference binding partner, and another reactive residue comprised within said peptide tag, wherein an increased binding efficacy is at least one of the total binding and the binding rate;
b) increased ability to form a particle displaying a peptide of interest, such as a virus-like particle displaying a peptide of interest, wherein the particle comprises a particle-forming protein such as a virus-like particle-forming protein fused to the modified binding partner and the peptide of interest fused to the peptide tag, or wherein the particle comprises the particle-forming protein fused to the peptide tag and the peptide of interest fused to the modified binding partner, and wherein the particle is formed by spontaneous formation of the isopeptide bond between the modified binding partner and the peptide tag, when compared to the ability of the reference binding partner to form a particle under similar conditions;
c) increased ability to display a peptide of interest on a particle such as a virus-like particle, wherein the particle comprises a particle-forming protein such as a virus-like particle-forming protein fused to the modified binding partner and the peptide of interest fused to the peptide tag, or wherein the particle comprises the particle-forming protein fused to the peptide tag and the peptide of interest fused to the modified binding partner, and wherein the particle is formed by spontaneous formation of the isopeptide bond between the modified binding partner and the peptide tag, when compared to the ability of the reference binding partner to display the peptide of interest under similar conditions.
29. The modified binding partner according to item 28, wherein the particle induces a greater immune response than a particle formed with a reference binding partner but otherwise identical when administered to a subject in need thereof, preferably wherein the reference binding partner is SpyCatcher (SEQ ID NO: 1), optionally wherein the increased immune response is an increased IgM response and/or an increased IgG2 response, such as an increased IgG2a and/or IgG2b.
30. The modified binding partner according to any one of items 28 to 29, comprising or consisting of SEQ ID NO: 37, SEQ ID NO: 39, SEQ ID NO: 29, SEQ ID NO: 71 or SEQ ID NO: 41, or a fragment or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto.
31. A peptide tag having one or more improved properties compared to a reference peptide tag, wherein the one or more improved properties are independently selected from one or more of:
a) increased binding efficacy of the peptide tag to a reference binding partner relative to the binding of the reference peptide tag to said reference binding partner, wherein said peptide tag and said reference peptide tag are capable of binding to said reference binding partner via the spontaneous formation of an isopeptide bond between one reactive residue comprised within said peptide tag or within said reference peptide tag, and another reactive residue comprised within said reference binding partner, wherein the binding efficacy is increased if at least one of the total binding and the binding rate is increased;
b) increased ability to form a particle such as a virus-like particle, wherein the particle displays a peptide of interest, wherein the particle comprises a particle-forming protein such as a virus-like particle-forming protein fused to the reference binding partner and the peptide of interest fused to the peptide tag, or wherein the particle comprises the particle-forming protein fused to the peptide tag and the peptide of interest fused to the reference binding partner, and wherein the particle is formed by spontaneous formation of the isopeptide bond between the reference binding partner and the peptide tag, when compared to the ability of the reference peptide tag to form a particle under similar conditions;
c) increased ability to display a compound of interest such as a peptide on a particle such as a virus-like particle, wherein the particle comprises a particle-forming protein such as a virus-like particle-forming protein fused to the peptide tag and the compound of interest fused to the binding partner, or wherein the particle comprises the particle-forming protein fused to the binding partner and the compound of interest fused to the peptide tag, and wherein the particle is formed by spontaneous formation of the isopeptide bond between the binding partner and the peptide tag, when compared to the ability of the reference peptide tag to display the compound of interest under similar conditions.
32. The peptide tag according to item 31, wherein the particle induces a greater immune response than a particle formed with a reference peptide tag but otherwise identical when administered to a subject in need thereof, optionally wherein the increased immune response is an increased IgM response and/or an increased IgG2 response, such as an increased IgG2a and/or IgG2b.
33. The peptide tag according to any one of items 31 to 32, comprising or consisting of SEQ ID NO: 46 (RumTag), SEQ ID NO: 47 (RumTrunkD9NTag), SEQ ID NO: 50 (PhoTag), SEQ ID NO: 52 (EntTag), SEQ ID NO: 54 (Rum7Tag), SEQ ID NO: 56 (Rum3Tag), SEQ ID NO: 58 (Rum2Tag), SEQ ID NO: 60 (Rum4Tag), SEQ ID NO: 62 (Rum5Tag), SEQ ID NO: 64 (Rum6Tag), SEQ ID NO: 66 (BacTag), SEQ ID NO: 68 (Bac2Tag), SEQ ID NO: 35 (Bac3Tag), SEQ ID NO: 22 (Bac4Tag), SEQ ID NO: 29, SEQ ID NO: 31 and SEQ ID NO: 12 (Bac5Tag) and homologues thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto.
34. A peptide pair comprising or consisting of a peptide tag and a binding partner, wherein the peptide pair has one or more improved properties compared to a reference peptide pair comprising a reference peptide tag and a reference binding partner,
   wherein the binding partner is capable of binding to the peptide tag via the spontaneous formation of an isopeptide bond between one reactive residue comprised within said modified binding partner, and another reactive residue comprised within said peptide tag;
   wherein the reference binding partner is capable of binding to the reference peptide tag via the spontaneous formation of an isopeptide bond between one reactive residue comprised within said reference binding partner, and another reactive residue comprised within said reference peptide tag;
   wherein the one or more improved properties are independently selected from one or more of:
   a) Increased binding efficacy of the binding partner to the peptide tag relative to the binding of the reference binding partner to the reference peptide tag, wherein the binding efficacy is increased if at least one of the total binding and the binding rate is increased;
   b) Increased ability to form a particle displaying a peptide of interest, such as a virus-like particle displaying a peptide of interest, wherein the particle comprises a particle-forming protein such as a virus-like particle-forming protein fused to the binding partner and the compound of interest fused to the peptide tag, or wherein the particle comprises the virus-like particle-forming protein fused to the peptide tag and the compound of interest fused to the binding partner, and wherein the particle is formed by spontaneous formation of the isopeptide bond between the binding partner and the peptide tag, when compared to the ability of the reference peptide pair to form a particle under similar conditions; and/or
   c) increased ability to display a compound of interest such as a peptide on a particle such as a virus-like particle, wherein the particle comprises a particle-forming protein such as a virus-like particle-forming protein fused to the binding partner and the compound of interest fused to the peptide tag, or wherein the particle comprises the particle-forming protein fused to the peptide tag and the compound of interest fused to the binding partner, and wherein the particle is formed by spontaneous formation of the isopeptide bond between the binding partner and the peptide tag, when compared to the ability of the reference peptide pair to display the peptide of interest under similar conditions.
35. The peptide pair according to item 34, wherein the particle induces a greater immune response than a particle formed with a reference peptide pair but otherwise identical when administered to a subject in need thereof, optionally wherein the increased immune response is an increased IgM response and/or an increased IgG2 response, such as an increased IgG2a and/or IgG2b, preferably wherein the reference peptide pair comprises SpyCatcher (SEQ ID NO: 1).
36. The peptide pair according to any one of items 34 to 35, wherein the binding partner is a modified binding partner as defined in any one of the preceding items, and wherein:
   a) If the modified binding partner comprises or consists of the first reactive fragment of the first binding partner and the second residual fragment of the second binding partner as defined in any one of the preceding items, the reference binding partner is the second binding partner;
   b) If the modified binding partner comprises or consists of the second reactive fragment of the second binding partner and the first residual fragment of the first binding partner as defined in any one of the preceding items, the reference binding partner is the first binding partner.
37. The modified binding partner, the peptide tag or the peptide pair according to any one of items 9 to 17, 22 to 24 or 26 to 35, wherein the particle is a virus-like particle.
38. The modified binding partner, the peptide tag or the peptide pair according to any one of items 9 to 17, 22 to 24 or 26 to 35, wherein the particle is a virus-like particle and the particle-forming protein is a virus capsid protein.
39. The modified binding partner, the peptide tag or the peptide pair according to any one of items 9 to 17, 22 to 24 or 26 to 35, wherein the virus capsid protein is selected from the group consisting of: an AP205 capsid protein, an MS2 capsid protein, an HBc capsid protein, and a phage fr capsid protein.
40. A polynucleotide encoding the modified partner according to any one of items 9 to 17 or 28 to 29 and/or the peptide tag according to any one of items 22 to 24 or 31.
41. The polynucleotide according to item 40, encoding:
   a) the modified binding partner as set forth in SEQ ID NO: 37, said polynucleotide comprising or consisting of SEQ ID NO: 38;
   b) the modified binding partner as set forth in SEQ ID NO: 39, said polynucleotide comprising or consisting of SEQ ID NO: 40; or
   c) the modified binding partner as set forth in SEQ ID NO: 41, said polynucleotide comprising or consisting of SEQ ID NO: 42;
   d) the modified binding partner as set forth in SEQ ID NO: 29, said polynucleotide comprising or consisting of SEQ ID NO: 30;
   e) the modified binding partner as set forth in SEQ ID NO: 71, said polynucleotide comprising or consisting of SEQ ID NO: 72,
      or homologues thereof having at least 60% identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% identity thereto.
42. The polynucleotide according to any one of items 40 to 41, encoding:
   a) the peptide tag as set forth in SEQ ID NO: 46, said polynucleotide comprising or consisting of SEQ ID NO: 45;
   b) the peptide tag as set forth in SEQ ID NO: 48, said polynucleotide comprising or consisting of SEQ ID NO: 47;
   c) the peptide tag as set forth in SEQ ID NO: 50, said polynucleotide comprising or consisting of SEQ ID NO: 49;
   d) the peptide tag as set forth in SEQ ID NO: 52, said polynucleotide comprising or consisting of SEQ ID NO: 51;
   e) the peptide tag as set forth in SEQ ID NO: 54, said polynucleotide comprising or consisting of SEQ ID NO: 53;
   f) the peptide tag as set forth in SEQ ID NO: 56, said polynucleotide comprising or consisting of SEQ ID NO: 55;
   g) the peptide tag as set forth in SEQ ID NO: 58, said polynucleotide comprising or consisting of SEQ ID NO: 57;
   h) the peptide tag as set forth in SEQ ID NO: 60, said polynucleotide comprising or consisting of SEQ ID NO: 59;
   i) the peptide tag as set forth in SEQ ID NO: 62, said polynucleotide comprising or consisting of SEQ ID NO: 61;
   j) the peptide tag as set forth in SEQ ID NO: 64, said polynucleotide comprising or consisting of SEQ ID NO: 63;
   k) the peptide tag as set forth in SEQ ID NO: 66, said polynucleotide comprising or consisting of SEQ ID NO: 65;
   l) the peptide tag as set forth in SEQ ID NO: 68, said polynucleotide comprising or consisting of SEQ ID NO: 67;
   m) the peptide tag as set forth in SEQ ID NO: 35, said polynucleotide comprising or consisting of SEQ ID NO: 36;
   n) the peptide tag as set forth in SEQ ID NO: 22, said polynucleotide comprising or consisting of SEQ ID NO: 21;
   o) the peptide tag as set forth in SEQ ID NO: 12, said polynucleotide comprising or consisting of SEQ ID NO: 11;
   p) the peptide tag as set forth in SEQ ID NO: 31, said polynucleotide comprising or consisting of SEQ ID NO: 32,
      or homologues thereof having at least 60% identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% identity thereto.
43. A vector comprising a polynucleotide according to any one of items 40 to 42.
44. A host cell expressing:
i) The modified binding partner according to any one of items 9 to 17 or 28 to 29, and/or
ii) The peptide tag according to any one of items 22 to 23 or 31.
45. The host cell according to item 44, wherein the cell is a bacterial cell, a yeast cell, a fungal cell, a plant cell, a mammalian cell or an insect cell.
46. A composition comprising:
i) A protein fused to a modified binding partner according to any one of items 9 to 17 or 28 to 29, and a compound of interest such as a peptide, for example an antigen, fused to a peptide tag according to any one of items 22 to 23 or 31; or
ii) A protein fused to a peptide tag according to any one of items 22 to 23 or 31, and a compound of interest such as a peptide, for example an antigen, fused to a modified binding partner according to any one of items 9 to 17 or 28 to 29,
wherein the modified binding partner and the peptide tag are capable of interacting by the spontaneous formation of an isopeptide bond, and
wherein the compound of interest and the protein are linked via an isopeptide bond between the modified binding partner and the peptide tag.
47. The composition according to item 46, wherein the protein fused to the modified binding partner is a particle-forming protein.
48. The composition according to item 47, wherein the particle-forming protein and the compound of interest form a particle displaying said compound of interest.
49. The composition according to item 48, wherein the particle is a virus-like particle and the particle-forming protein is a virus capsid protein.
50. The composition according to any one of items 46 to 47, wherein the peptide of interest is an antigen, preferably wherein the antigen is a peptide or an antigenic fragment thereof, such as a peptide or an antigenic fragment thereof which is associated with an abnormal physiological response.
51. The composition according to any one of items 46 to 50, wherein the abnormal physiological response is a disease such as a cardiovascular disease, an infectious disease and/or an allergic reaction/disease.
52. The composition according to any one of items 46 to 51, wherein the disease is cancer, such as breast cancer, gastric cancer, ovarian cancer and uterine serous carcinoma; a cardiovascular disease, such as dyslipidemia, atherosclerosis, and/or hypercholesterolemia; an immune-inflammatory disease or a chronic disease, such as eosinophilic asthma, allergy, nasal polyposis, atopic dermatitis, eosinophilic esophagitis, hypereosinophilic syndrome, and Churg-Strauss syndrome, a neurological disease such as Alzheimer's disease; an infectious disease, such as an infectious disease selected from the group consisting of diseases caused by a virus, such as a coronavirus, for example SARS-CoV-2, malaria, tuberculosis, HIV and influenza; a lipid disorder such as hyperlipidemia, type I, type II, type III, type IV, or type V hyperlipidemia, secondary hypertriglyceridemia, hypercholesterolemia, familial hypercholesterolemia, xanthomatosis, cholesterol acetyltransferase deficiency; an arteriosclerotic condition such as atherosclerosis; or a coronary artery disease.
53. The composition according to any one of items 46 to 52, wherein the antigen is a protein, peptide and/or an antigenic fragment from the group consisting of cancer-specific polypeptides, polypeptides associated with cardiovascular diseases, polypeptides associated with asthma, polypeptides associated with nasal polyposis, polypeptides associated with atopic dermatitis, polypeptides associated with eosinophilic esophagitis, polypeptides associated with hypereosinophilic syndrome, polypeptides associated with Churg-Strauss syndrome and/or polypeptides associated with pathogenic organisms.
54. The composition according to any one of items 46 to 53, the polynucleotide according to any one of items 40 to 42 or the vector according to item 43, for use in the prophylaxis or treatment of a disease.
55. The composition, the polynucleotide or the vector for the use according to item 53, wherein the disease is cancer, such as breast cancer, gastric cancer, ovarian cancer and uterine serous carcinoma; a cardiovascular disease, such as dyslipidemia, atherosclerosis, and/or hypercholesterolemia; an immune-inflammatory disease or a chronic disease, such as eosinophilic asthma, allergy, nasal polyposis, atopic dermatitis, eosinophilic esophagitis, hypereosinophilic syndrome, and Churg-Strauss syndrome, a neurological disease such as Alzheimer's disease; an infectious disease, such as an infectious disease selected from the group consisting of diseases caused by a virus, such as a coronavirus, for example SARS-CoV-2, malaria, tuberculosis, HIV and influenza; a lipid disorder such as hyperlipidemia, type I, type II, type III, type IV, or type V hyperlipidemia, secondary hypertriglyceridemia, hypercholesterolemia, familial hypercholesterolemia, xanthomatosis, cholesterol acetyltransferase deficiency; an arteriosclerotic condition such as atherosclerosis; or a coronary artery disease.
56. The composition according to any one of items 46 to 53, the polynucleotide according to any one of items 40 to 42 or the vector according to item 43, for use in a method for inducing an immune response in a subject, the method comprising the step of administering said composition, polynucleotide or vector to said subject at least once.
57. A method of manufacturing a pharmaceutical composition according to any one of items any one of items 46 to 56, comprising the steps of:
i) obtaining a first polypeptide comprising or consisting of a modified binding partner according to any one of items 9 to 17 or 28 to 29 fused to a protein; and obtaining a second polypeptide comprising or consisting of a peptide tag as defined in any one of items 22 to 24 or 31 fused to a compound of interest; or
   obtaining a first polypeptide comprising or consisting of a peptide tag as defined in any one of items 22 to 24 or 31 fused to a protein and obtaining a second polypeptide comprising or consisting of a modified binding partner according to any one of items 9 to 17 or 28 to 29 fused to a compound of interest;
ii) contacting the first polypeptide and the second polypeptide, thereby allowing formation of an isopeptide bond between the peptide tag and the modified binding partner; and generating a pharmaceutical composition according to any one of items 46 to 56.
58. The method according to item 57, wherein the protein is a particle-forming protein.

1. A peptide pair consisting of a peptide tag and a binding partner, wherein the peptide tag and the binding partner can bind to one another via the spontaneous formation of an isopeptide bond between one reactive residue comprised within said binding partner and another reactive residue comprised within said peptide tag, wherein the peptide pair is selected from:
a) the binding partner of SEQ ID NO: 39 (MoonCake) and a peptide tag selected from SEQ ID NO: 47 (RumTrunkD9NTag), SEQ ID NO: 54 (Rum7Tag), SEQ ID NO: 56 (Rum3Tag), SEQ ID NO: 58 (Rum2Tag), SEQ ID NO: 60 (Rum4Tag), SEQ ID NO: 62 (Rum5Tag), SEQ ID NO: 64 (Rum6Tag), SEQ ID NO: 71 (RumTrunkTag), SEQ ID NO: 5 (SpyTag), SEQ ID NO: 7 (SdyTag), SEQ ID NO: 46 (RumTag), SEQ ID NO: 66 (BacTag); SEQ ID NO: 68 (Bac2Tag), SEQ ID NO: 35 (Bac3Tag), SEQ ID NO: 22 (Bac4Tag) and SEQ ID NO: 12 (Bac5Tag); or variants thereof having at least 70% homology or identity thereto;
b) the binding partner of SEQ ID NO: 41 (KatI) and a peptide tag selected from SEQ ID NO: 47 (RumTrunkD9NTag), SEQ ID NO: 54 (Rum7Tag), SEQ ID NO: 56 (Rum3Tag), SEQ ID NO: 58 (Rum2Tag), SEQ ID NO: 60 (Rum4Tag), SEQ ID NO: 62 (Rum5Tag), SEQ ID NO: 64 (Rum6Tag), SEQ ID NO: 71 (RumTrunkTag), SEQ ID NO: 5 (SpyTag), SEQ ID NO: 7 (SdyTag), SEQ ID NO: 46 (RumTag), SEQ ID NO: 66 (BacTag); SEQ ID NO: 68 (Bac2Tag), SEQ ID NO: 35 (Bac3Tag), SEQ ID NO: 22 (Bac4Tag) and SEQ ID NO: 12 (Bac5Tag); or variants thereof having at least 70% homology or identity thereto; and
c) the binding partner of SEQ ID NO: 37 (QueenCatcher) and a peptide tag selected from SEQ ID NO: 47 (RumTrunkD9NTag), SEQ ID NO: 54 (Rum7Tag), SEQ ID NO: 56 (Rum3Tag), SEQ ID NO: 58 (Rum2Tag), SEQ ID NO: 60 (Rum4Tag), SEQ ID NO: 62 (Rum5Tag), SEQ ID NO: 64 (Rum6Tag), SEQ ID NO: 71 (RumTrunkTag), SEQ ID NO: 5 (SpyTag), SEQ ID NO: 7 (SdyTag), SEQ ID NO: 46 (RumTag), SEQ ID NO: 66 (BacTag); SEQ ID NO: 68 (Bac2Tag), SEQ ID NO: 35 (Bac3Tag), SEQ ID NO: 22 (Bac4Tag), SEQ ID NO: 12 (Bac5Tag) and SEQ ID NO: 76 (Clib9).

2. The peptide pair according to claim 1, wherein the peptide pair is selected from:
a) the binding partner of SEQ ID NO: 39 (MoonCake) and the peptide tag of SEQ ID NO: 47 (RumTrunkD9NTag); or variants thereof having at least 70% homology or identity thereto;
b) the binding partner of SEQ ID NO: 41 (KatI) and the peptide tag of SEQ ID NO: 47 (RumTrunkD9NTag); or variants thereof having at least 70% homology or identity thereto;
c) the binding partner of SEQ ID NO: 39 (MoonCake) and the peptide tag of SEQ ID NO: 46 (RumTag); or variants thereof having at least 70% homology or identity thereto; and
d) the binding partner of SEQ ID NO: 41 (KatI) and the peptide tag of SEQ ID NO: 46 (RumTag); or variants thereof having at least 70% homology or identity thereto.

3. A binding partner capable of binding to a peptide tag via the spontaneous formation of an isopeptide bond between one reactive residue comprised within said binding partner and another reactive residue comprised within said peptide tag, wherein the binding partner is selected from:
a) SEQ ID NO: 39 (MoonCake);
b) SEQ ID NO: 41 (KatI); and
c) SEQ ID NO: 37 (QueenCatcher);
or fragments or homologues thereof having at least 70% homology or identity thereto, with the proviso that said fragments or homologues comprise the reactive residue involved in the formation of the isopeptide bond or wherein where the reactive residue is an Aspartic acid (D), the reactive residue is mutated to an Asparagine (N).

4. The peptide pair according to claim 2, wherein the binding partner is:
a) SEQ ID NO: 39 or a fragment or a homologue of SEQ ID NO: 39 having at least 70% homology or identity to SEQ ID NO: 39, with the proviso that the residue corresponding to residue 31 in SEQ ID NO: 39 is not modified; or
b) SEQ ID NO: 37 or a fragment or a homologue of SEQ ID NO: 37 having at least 70% homology or identity to SEQ ID NO: 37, with the proviso that the residue corresponding to residue 31 in SEQ ID NO: 37 is not modified; or
c) SEQ ID NO: 41 or a fragment or a homologue of SEQ ID NO: 41 having at least 70% homology or identity to SEQ ID NO: 41, with the proviso that the residue corresponding to residue 31 in SEQ ID NO: 39 is not modified.

5. The peptide pair according to claim 2, wherein:
a) the binding partner is SEQ ID NO: 39 (MoonCake) or a fragment or a homologue thereof having at least 70% homology or identity thereto, with the proviso that the residue corresponding to residue 31 in SEQ ID NO: 39 is not modified, and can bind to a peptide tag via an isopeptide bond, wherein the peptide tag is selected from SEQ ID NO: 5 (SpyTag), SEQ ID NO: 7 (SdyTag), SEQ ID NO: 46 (RumTag), SEQ ID NO: 47 (RumTrunkD9NTag), SEQ ID NO: 54 (Rum7Tag), SEQ ID NO: 56 (Rum3Tag), SEQ ID NO: 58 (Rum2Tag), SEQ ID NO: 60 (Rum4Tag), SEQ ID NO: 62 (Rum5Tag), SEQ ID NO: 64 (Rum6Tag), SEQ ID NO: 71 (RumTrunkTag), SEQ ID NO: 66 (BacTag); SEQ ID NO: 68 (Bac2Tag), SEQ ID NO: 35 (Bac3Tag), SEQ ID NO: 22 (Bac4Tag) and SEQ ID NO: 12 (Bac5Tag); or
b) the binding partner SEQ ID NO: 41 (KatI) or a fragment or a homologue thereof having at least 70% homology or identity thereto, with the proviso that the residue corresponding to residue 31 in SEQ ID NO: 41 is not modified, and can bind via an isopeptide bond, wherein the peptide tag is selected from SEQ ID NO: 5 (SpyTag), SEQ ID NO: 7 (SdyTag), SEQ ID NO: 46 (RumTag), SEQ ID NO: 47 (RumTrunkD9NTag), SEQ ID NO: 54 (Rum7Tag), SEQ ID NO: 56 (Rum3Tag), SEQ ID NO: 58 (Rum2Tag), SEQ ID NO: 60 (Rum4Tag), SEQ ID NO: 62 (Rum5Tag), SEQ ID NO: 64 (Rum6Tag), SEQ ID NO: 71 (RumTrunkTag), SEQ ID NO: 66 (BacTag); SEQ ID NO: 68 (Bac2Tag), SEQ ID NO: 35 (Bac3Tag), SEQ ID NO: 22 (Bac4Tag) and SEQ ID NO: 12 (Bac5Tag); or
c) the binding partner is SEQ ID NO: 37 (QueenCatcher) or a fragment or a homologue thereof having at least 70% homology or identity thereto, with the proviso that the residue corresponding to residue 31 in SEQ ID NO: 37 is not modified, and can bind to a peptide tag via an isopeptide bond, wherein the peptide tag is selected from SEQ ID NO: 5 (SpyTag), SEQ ID NO: 7 (SdyTag), SEQ ID NO: 46 (RumTag), SEQ ID NO: 47 (RumTrunkD9NTag), SEQ ID NO: 54 (Rum7Tag), SEQ ID NO: 56 (Rum3Tag), SEQ ID NO: 58 (Rum2Tag), SEQ ID NO: 60 (Rum4Tag), SEQ ID NO: 62 (Rum5Tag), SEQ ID NO: 64 (Rum6Tag), SEQ ID NO: 71 (RumTrunkTag), SEQ ID NO: 66 (BacTag); SEQ ID NO: 68 (Bac2Tag), SEQ ID NO: 35 (Bac3Tag), SEQ ID NO: 22 (Bac4Tag), SEQ ID NO: 12 (Bac5Tag) and SEQ ID NO: 76 (Clib9).

6. The peptide pair according to claim 2, wherein:
a) the binding partner is SEQ ID NO: 39 (MoonCake) or a fragment or a homologue thereof having at least 70% homology or identity thereto, with the proviso that the residue corresponding to residue 31 in SEQ ID NO: 39 is not modified, and can bind to a peptide tag via an isopeptide bond, wherein the peptide tag is selected from SEQ ID NO: 46 (RumTag) and SEQ ID NO: 47 (RumTrunkD9NTag); or
b) the binding partner is SEQ ID NO: 41 (KatI) or a fragment or a homologue thereof having at least 70% homology or identity thereto, with the proviso that the residue corresponding to residue 31 in SEQ ID NO: 41 is not modified, and can bind via an isopeptide bond, wherein the peptide tag is selected from SEQ ID NO: 46 (RumTag) and SEQ ID NO: 47 (RumTrunkD9NTag); or
c) the binding partner is SEQ ID NO: 37 (QueenCatcher) or a fragment or a homologue thereof having at least 70% homology or identity thereto, with the proviso that the residue corresponding to residue 31 in SEQ ID NO: 37 is not modified, and can bind to a peptide tag via an isopeptide bond, wherein the peptide tag is selected from SEQ ID NO: 5 (SpyTag), SEQ ID NO: 7 (SdyTag), SEQ ID NO: 46 (RumTag), SEQ ID NO: 47 (RumTrunkD9NTag), SEQ ID NO: 71 (RumTrunkTag) and SEQ ID NO: 75 (Clib9).

7. A peptide tag capable of binding to a binding partner via the spontaneous formation of an isopeptide bond between one reactive residue comprised within said binding partner and another reactive residue comprised within said peptide tag, wherein the peptide tag is selected from:
a) SEQ ID NO: 47 (RumTrunkD9NTag);
b) SEQ ID NO: 46 (RumTag);
c) SEQ ID NO: 71 (RumTrunkTag);
d) SEQ ID NO: 58 (Rum2Tag);
e) SEQ ID NO: 56 (Rum3 Tag);
f) SEQ ID NO: 60 (Rum4Tag);
g) SEQ ID NO: 62 (Rum5Tag);
h) SEQ ID NO: 64 (Rum6Tag);
i) SEQ ID NO: 54 (Rum7Tag);
j) SEQ ID NO: 66 (BacTag);
k) SEQ ID NO: 68 (Bac2Tag);
l) SEQ ID NO: 35 (Bac3 Tag);
m) SEQ ID NO: 22 (Bac4Tag);
n) SEQ ID NO: 12 (Bac5Tag);
o) SEQ ID NO: 50 (PhoTag); and
p) SEQ ID NO: 52 (EntTag);
or a fragment or a homologue thereof having at least 70% homology or identity thereto, with the proviso that said fragments or homologues comprise the reactive residue involved in the formation of the isopeptide bond or wherein where the reactive residue is an Aspartic acid (D), the reactive residue is mutated to an Asparagine (N).

8. The peptide tag according to claim 7, wherein the peptide tag is:
a) SEQ ID NO: 47 (RumTrunkD9NTag) or a fragment or a homologue thereof having at least 70% homology or identity thereto, with the proviso that the residue corresponding to residue 9 in SEQ ID NO: 47 is not modified; or
b) SEQ ID NO: 46 (RumTag) or a fragment or a homologue thereof having at least 70% homology or identity thereto, with the proviso that the residue corresponding to residue 12 in SEQ ID NO: 46 is not modified;
c) SEQ ID NO: 71 (RumTrunkTag) or a fragment or a homologue thereof having at least 70% homology or identity thereto, with the proviso that the residue corresponding to residue 9 in SEQ ID NO: 71 is not modified.

9. The peptide tag according to claim 7, wherein the peptide tag is:
a) SEQ ID NO: 47 (RumTrunkD9NTag) or a fragment or a homologue of SEQ ID NO: 47 having at least 70% homology or identity to SEQ ID NO: 47, with the proviso that the residue corresponding to residue 9 in SEQ ID NO: 47 is not modified, wherein the binding partner is selected from SEQ ID NO: 37 (QueenCatcher), SEQ ID NO: 39 (MoonCake) and SEQ ID NO: 41 (KatI); or
b) SEQ ID NO: 46 (RumTag) or a fragment or a homologue of SEQ ID NO: 46 having at least 70% homology or identity to SEQ ID NO: 46, with the proviso that the residue corresponding to residue 12 in SEQ ID NO: 46 is not modified, and can bind to a binding partner via an isopeptide bond, wherein the binding partner is selected from SEQ ID NO: 37 (QueenCatcher), SEQ ID NO: 39 (MoonCake) and SEQ ID NO: 41 (KatI); or
c) SEQ ID NO: 71 (RumTrunkTag) or a fragment or a homologue of SEQ ID NO: 71 having at least 70% homology or identity to SEQ ID NO: 71, with the proviso that the residue corresponding to residue 9 in SEQ ID NO: 71 is not modified, wherein the binding partner is selected from SEQ ID NO: 37 (QueenCatcher), SEQ ID NO: 39 (MoonCake) and SEQ ID NO: 41 (KatI).

10. A polynucleotide encoding the binding partner according to claim 3.

11. The polynucleotide according to claim 10, wherein the polynucleotide encodes a binding partner selected from:
a) SEQ ID NO: 39 (MoonCake);
b) SEQ ID NO: 41 (KatI); and
c) SEQ ID NO: 37 (QueenCatcher);
or fragments or homologues thereof having at least 70% homology or identity thereto, with the proviso that said fragments or homologues comprise the reactive residue involved in the formation of the isopeptide bond or wherein where the reactive residue is an Aspartic acid (D), the reactive residue is mutated to an Asparagine (N).

12. The polynucleotide according to claim 10, wherein the polynucleotide is selected from:
a) SEQ ID NO: 40 (MoonCake) or a homologue thereof having at least 70% homology or identity thereto, with the proviso that the codon at position 91 to 93 of SEQ ID NO: 40 encodes a lysine; or
b) SEQ ID NO: 42 (KatI) or a homologue thereof having at least 70% homology or identity thereto, wherein the proviso that the codon at position 91 to 93 of SEQ ID NO: 42 encodes a lysine; or
c) SEQ ID NO: 38 (QueenCatcher) or a homologue thereof having at least 70% homology or identity thereto, with the proviso that the codon at position 91 to 93 of SEQ ID NO: 38 encodes a lysine.

13.-33. (canceled)

34. The binding partner according to claim 3, wherein the binding partner is:
a) SEQ ID NO: 39 or a fragment or a homologue of SEQ ID NO: 39 having at least 70% homology or identity to SEQ ID NO: 39, with the proviso that the residue corresponding to residue 31 in SEQ ID NO: 39 is not modified; or
b) SEQ ID NO: 37 or a fragment or a homologue of SEQ ID NO: 37 having at least 70% homology or identity to SEQ ID NO: 37, with the proviso that the residue corresponding to residue 31 in SEQ ID NO: 37 is not modified; or
c) SEQ ID NO: 41 or a fragment or a homologue of SEQ ID NO: 41 having at least 70% homology or identity to SEQ ID NO: 41, with the proviso that the residue corresponding to residue 31 in SEQ ID NO: 39 is not modified.

35. The binding partner according to claim 3, wherein:
a) the binding partner is SEQ ID NO: 39 (MoonCake) or a fragment or a homologue thereof having at least 70% homology or identity thereto, with the proviso that the residue corresponding to residue 31 in SEQ ID NO: 39 is not modified, and can bind to a peptide tag via an isopeptide bond, wherein the peptide tag is selected from SEQ ID NO: 5 (SpyTag), SEQ ID NO: 7 (SdyTag), SEQ ID NO: 46 (RumTag), SEQ ID NO: 47 (RumTrunkD9NTag), SEQ ID NO: 54 (Rum7Tag), SEQ ID NO: 56 (Rum3Tag), SEQ ID NO: 58 (Rum2Tag), SEQ ID NO: 60 (Rum4Tag), SEQ ID NO: 62 (Rum5Tag), SEQ ID NO: 64 (Rum6Tag), SEQ ID NO: 71 (RumTrunkTag), SEQ ID NO: 66 (BacTag); SEQ ID NO: 68 (Bac2Tag), SEQ ID NO: 35 (Bac3Tag), SEQ ID NO: 22 (Bac4Tag) and SEQ ID NO: 12 (Bac5Tag); or
b) the binding partner SEQ ID NO: 41 (KatI) or a fragment or a homologue thereof having at least 70% homology or identity thereto, with the proviso that the residue corresponding to residue 31 in SEQ ID NO: 41 is not modified, and can bind via an isopeptide bond, wherein the peptide tag is selected from SEQ ID NO: 5 (SpyTag), SEQ ID NO: 7 (SdyTag), SEQ ID NO: 46 (RumTag), SEQ ID NO: 47 (RumTrunkD9NTag), SEQ ID NO: 54 (Rum7Tag), SEQ ID NO: 56 (Rum3Tag), SEQ ID NO: 58 (Rum2Tag), SEQ ID NO: 60 (Rum4Tag), SEQ ID NO: 62 (Rum5Tag), SEQ ID NO: 64 (Rum6Tag), SEQ ID NO: 71 (RumTrunkTag), SEQ ID NO: 66 (BacTag); SEQ ID NO: 68 (Bac2Tag), SEQ ID NO: 35 (Bac3Tag), SEQ ID NO: 22 (Bac4Tag) and SEQ ID NO: 12 (Bac5Tag); or
c) the binding partner is SEQ ID NO: 37 (QueenCatcher) or a fragment or a homologue thereof having at least 70% homology or identity thereto, with the proviso that the residue corresponding to residue 31 in SEQ ID NO: 37 is not modified, and can bind to a peptide tag via an isopeptide bond, wherein the peptide tag is selected from SEQ ID NO: 5 (SpyTag), SEQ ID NO: 7 (SdyTag), SEQ ID NO: 46 (RumTag), SEQ ID NO: 47 (RumTrunkD9NTag), SEQ ID NO: 54 (Rum7Tag), SEQ ID NO: 56 (Rum3Tag), SEQ ID NO: 58 (Rum2Tag), SEQ ID NO: 60 (Rum4Tag), SEQ ID NO: 62 (Rum5Tag), SEQ ID NO: 64 (Rum6Tag), SEQ ID NO: 71 (RumTrunkTag), SEQ ID NO: 66 (BacTag); SEQ ID NO: 68 (Bac2Tag), SEQ ID NO: 35 (Bac3Tag), SEQ ID NO: 22 (Bac4Tag), SEQ ID NO: 12 (Bac5Tag) and SEQ ID NO: 76 (Clib9).

36. The binding partner according to claim 3, wherein:
a) the binding partner is SEQ ID NO: 39 (MoonCake) or a fragment or a homologue thereof having at least 70% homology or identity thereto, with the proviso that the residue corresponding to residue 31 in SEQ ID NO: 39 is not modified, and can bind to a peptide tag via an isopeptide bond, wherein the peptide tag is selected from SEQ ID NO: 46 (RumTag) and SEQ ID NO: 47 (RumTrunkD9NTag); or
b) the binding partner is SEQ ID NO: 41 (KatI) or a fragment or a homologue thereof having at least 70% homology or identity thereto, with the proviso that the residue corresponding to residue 31 in SEQ ID NO: 41 is not modified, and can bind via an isopeptide bond, wherein the peptide tag is selected from SEQ ID NO: 46 (RumTag) and SEQ ID NO: 47 (RumTrunkD9NTag); or
c) the binding partner is SEQ ID NO: 37 (QueenCatcher) or a fragment or a homologue thereof having at least 70% homology or identity thereto, with the proviso that the residue corresponding to residue 31 in SEQ ID NO: 37 is not modified, and can bind to a peptide tag via an isopeptide bond, wherein the peptide tag is selected from SEQ ID NO: 5 (SpyTag), SEQ ID NO: 7 (SdyTag), SEQ ID NO: 46 (RumTag), SEQ ID NO: 47 (RumTrunkD9NTag), SEQ ID NO: 71 (RumTrunkTag) and SEQ ID NO: 75 (Clib9).

37. A polynucleotide encoding the peptide tag according to claim 7.

38. The polynucleotide according to claim 37, wherein the polynucleotide encodes a peptide tag selected from:
a) SEQ ID NO: 47 (RumTrunkD9NTag);
b) SEQ ID NO: 46 (RumTag);
c) SEQ ID NO: 71 (RumTrunkTag);
d) SEQ ID NO: 58 (Rum2Tag);
e) SEQ ID NO: 56 (Rum3Tag);
f) SEQ ID NO: 60 (Rum4Tag);
g) SEQ ID NO: 62 (Rum5Tag);
h) SEQ ID NO: 64 (Rum6Tag);
i) SEQ ID NO: 54 (Rum7Tag);
j) SEQ ID NO: 66 (BacTag);
k) SEQ ID NO: 68 (Bac2Tag);
l) SEQ ID NO: 35 (Bac3 Tag);
m) SEQ ID NO: 22 (Bac4Tag); and
n) SEQ ID NO: 12 (Bac5Tag);
or a fragment or a homologue thereof having at least 70% homology or identity thereto, with the proviso that said fragments or homologues comprise the reactive residue involved in the formation of the isopeptide bond or wherein where the reactive residue is an Aspartic acid (D), the reactive residue is mutated to an Asparagine (N).

39. The polynucleotide according to claim 38, wherein the polynucleotide is selected from:
a) SEQ ID NO: 48 (RumTrunkD9NTag), or a homologue thereof having at least 70% homology or identity thereto, with the proviso that the codon at position 25 to 27 of SEQ ID NO: 48 encodes an asparagine;
b) SEQ ID NO: 45 (RumTag), or a homologue thereof having at least 70% homology or identity thereto, with the proviso that the codon at position 37 to 39 of SEQ ID NO: 45 encodes an aspartic acid;
c) SEQ ID NO: 72 (RumTrunkTag), or a homologue thereof having at least 70% homology or identity thereto, with the proviso that the codon at position 25 to 27 of SEQ ID NO:
72 encodes an aspartic acid;
d) SEQ ID NO: 57 (Rum2Tag) or a homologue thereof having at least 70% homology or identity thereto;
e) SEQ ID NO: 55 (Rum3Tag) or a homologue thereof having at least 70% homology or identity thereto;
f) SEQ ID NO: 59 (Rum4Tag) or a homologue thereof having at least 70% homology or identity thereto;
g) SEQ ID NO: 61 (Rum5Tag) or a homologue thereof having at least 70% homology or identity thereto;
h) SEQ ID NO: 63 (Rum6Tag) or a homologue thereof having at least 70% homology or identity thereto;
i) SEQ ID NO: 53 (Rum7Tag) or a homologue thereof having at least 70% homology or identity thereto;
j) SEQ ID NO: 65 (BacTag) or a homologue thereof having at least 70% homology or identity thereto;
k) SEQ ID NO: 67 (Bac2Tag) or a homologue thereof having at least 70% homology or identity thereto, with the proviso that the codon at position 37 to 39 encodes an aspartic acid;
l) SEQ ID NO: 36 (Bac3Tag) or a homologue thereof having at least 70% homology or identity thereto;
m) SEQ ID NO: 21 (Bac4Tag) or a homologue thereof having at least 70% homology or identity thereto; and
n) SEQ ID NO: 11 (Bac5Tag) or a homologue thereof having at least 70% homology or identity thereto;
or a fragment or a homologue thereof having at least 70% homology or identity thereto, with the proviso that said fragments or homologues comprise the reactive residue involved in the formation of the isopeptide bond or wherein where the reactive residue is an Aspartic acid (D), the reactive residue is mutated to an Asparagine (N).

40. The peptide pair according to claim 1, wherein the peptide tag is:
a) SEQ ID NO: 47 (RumTrunkD9NTag) or a fragment or a homologue thereof having at least 70% homology or identity thereto, with the proviso that the residue corresponding to residue 9 in SEQ ID NO: 47 is not modified; or
b) SEQ ID NO: 46 (RumTag) or a fragment or a homologue thereof having at least 70% homology or identity thereto, with the proviso that the residue corresponding to residue 12 in SEQ ID NO: 46 is not modified;
c) SEQ ID NO: 71 (RumTrunkTag) or a fragment or a homologue thereof having at least 70% homology or identity thereto, with the proviso that the residue corresponding to residue 9 in SEQ ID NO: 71 is not modified.

41. The peptide pair according to claim 1, wherein the peptide tag is:
a) SEQ ID NO: 47 (RumTrunkD9NTag) or a fragment or a homologue of SEQ ID NO: 47 having at least 70% homology or identity to SEQ ID NO: 47, with the proviso that the residue corresponding to residue 9 in SEQ ID NO: 47 is not modified, wherein the binding partner is selected from SEQ ID NO: 37 (QueenCatcher), SEQ ID NO: 39 (MoonCake) and SEQ ID NO: 41 (KatI); or
b) SEQ ID NO: 46 (RumTag) or a fragment or a homologue of SEQ ID NO: 46 having at least 70% homology or identity to SEQ ID NO: 46, with the proviso that the residue corresponding to residue 12 in SEQ ID NO: 46 is not modified, and can bind to a binding partner via an isopeptide bond, wherein the binding partner is selected from SEQ ID NO: 37 (QueenCatcher), SEQ ID NO: 39 (MoonCake) and SEQ ID NO: 41 (KatI); or
c) SEQ ID NO: 71 (RumTrunkTag) or a fragment or a homologue of SEQ ID NO: 71 having at least 70% homology or identity to SEQ ID NO: 71, with the proviso that the residue corresponding to residue 9 in SEQ ID NO: 71 is not modified, wherein the binding partner is selected from SEQ ID NO: 37 (QueenCatcher), SEQ ID NO: 39 (MoonCake) and SEQ ID NO: 41 (KatI).
